(19) United States
(12) Patent Application Publication
(10) Pub. No.: US 2024/0342270 A1
(43) Pub. Date: Oct. 17, 2024

(54) PROTECTIVE IMMUNITY ENHANCED ATTENUATED SALMONELLA VACCINE (PIESV) VECTOR SYSTEMS

(71) Applicant: UNIVERSITY OF FLORIDA RESEARCH FOUNDATION, INCORPORATED, Gainesville, FL (US)

(72) Inventors: Roy CURTISS, III, Gainesville, FL (US); Soo-Young C. WANDA, Gainesville, FL (US); Shifeng WANG, Gainesville, FL (US)

(21) Appl. No.: 18/265,323

(22) PCT Filed: Dec. 3, 2021
(86) PCT No.: PCT/US2021/061814

§ 371 Date: Jun. 5, 2023

Related U.S. Application Data
(60) Provisional application No. 63/120,940, filed on Dec. 3, 2020.

(51) Int. Cl. A61K 39/215; A61K 39/00; A61P 37/04; C07K 14/005; C12N 1/20; C12N 15/74

(52) U.S. Cl. CPC A61K 39/215; A61P 37/04; C07K 14/005; C12N 1/20; C12N 15/74; A61K 2039/522; A61K 2039/523; A61K 2039/53; C07K 2319/00

(57) ABSTRACT

Described within is a new much improved host-vector systems for delivery of synthesized antigens or of DNA vaccines to a diversity of animal and human hosts to elicit immune responses, especially protective immune responses to control infection and disease induction and/or transmission by bacterial, viral, parasite and fungal infectious disease agents.

### GOVERNMENT SUPPORT

This invention was made in part with government support under 2017-67017-26179 awarded by United States Department of Agriculture, NIFA and AI1056289 awarded by The National Institutes of Health. The government has certain rights in the invention.

### BACKGROUND

In the early 1980s, we selected attenuated derivatives of Salmonella enterica serotypes to serve as vaccines to deliver pathogen antigens specified by cloned genes to protect from infection by the pathogen whose antigens were synthesized and delivered to a vaccinated animal host (1, 2). We have continued since that time to improve these technologies of using Recombinant Attenuated Salmonella Vaccine (RASV) vectors. Initial efforts were to devise better means of attenuation by inactivation of global regulatory genes cya and crp governing catabolite metabolism (3, 4) and phoP governing virulence (5, 6). As a means to exclude use of antibiotic-resistance genes to selectively maintain plasmid vectors encoding pathogen antigens, a balanced-lethal vector-host system was invented in which mutations that blocked synthesis of peptidoglycan precursors whose absence leads to bacterial cell lysis were complemented with the wild-type gene specifying synthesis of those precursors on the plasmid vectors (7-9).

Improvements in maximizing induction of immune responses to delivered protective pathogen antigens were achieved by fusions that enable production of virus-like particles (VLPs) (10) or facilitated protective antigen secretion (11). Since attenuation is invariably associated with reduced immunogenicity (12), RASVs were genetically modified to display regulated delayed attenuation so that vaccine cells would display the invasive and colonizing ability of the wild-type parental strain at the time of mucosal vaccination and then gradually lose virulence attributes as a consequence of cell divisions in vivo as vaccine cells colonized internal effector lymphoid tissues (13-15). Similarly, synthesis of the protective antigens encoded by cloned genes also caused a metabolic load on the RASV to slow its growth and decrease colonizing ability. A means for regulated delayed antigen synthesis was thus devised (16, 17) so that antigen synthesis in vivo increased gradually as a consequence of cell division of the RASV with dilution of the repressor blocking antigen synthesis at the time of vaccination. Since there has always been concern for the survival of live attenuated bacterial vaccines if shed into the environment, a means for regulated delayed lysis in vivo was devised such that vaccines could not persist in vivo or survive if shed into the environment (18).

It was subsequently learned that RASVs with the regulated delayed lysis in vivo phenotype compared to RASVs that did not lyse induced enhanced levels of mucosal, systemic and cellular immunities to delivered antigens that could be further augmented by type 2 (T2) and type 3 (T3) secretion of protective antigens as well as by synthesis of VLPs prior to lysis (19-23). Further improvements were achieved by eliminating means by which Salmonella could suppress induction of immunity (24) with further improvements by eliminating ability of vaccine cells to synthesize polysaccharide capsular material that facilitated biofilms, decreased completeness of regulated lysis and suppressed antibody production (25). Further safety was achieved by devising additional means to cease synthesis of the LPS O-antigen in vivo that enhanced development of sensitivity to complement-mediated cytotoxicity and increased phagocytosis by macrophages (26-28). Collectively, these attributes have been superior in inducing protective immunity to the pathogens to which the vaccines are designed to protect against, but this has often required two immunizations. In addition, these improved vaccines are not inducing as high levels of protective immunity to Salmonella as did earlier RASV constructions.

### BRIEF DESCRIPTION OF THE DRAWINGS

FIG. 1. Plasmid maps. Regulated Delayed Lysis in vivo vectors (A) pG8R110, p15A ori with T3SS, (B) pG8R111, pBR ori; (pYA4594 pUC ori; pYA4589 p15A ori; and pYA4595, pSC101 ori; —not shown) (C) pG8R114, pBR ori; (pG8R113, p15A ori; and pG8R112, pSC101 ori, —not shown) all with the improved bla SS; (D) DNA vaccine vector pYA4545, pUC ori.

FIG. 2. Diagrammatic structure of Salmonella lipid A and the listing of gene products that modify its structure and the removal of the 1′ phosphate by expression of a codon-optimized version of the Francisella tularensis lpxE gene.

FIG. 3. Evaluation of S. typhimurium mutant strains for endotoxin activity using the Pierce Endotoxin Quantitation Kit.

FIG. 4. Western blot with anti-FliC180 (N-terminal) antibodies that also interact with the N-terminal segment of the FljB flagellin.

FIG. 5. Motility of all the mutant stains evaluated for synthesis of flagellins in FIG. 4.

FIG. 6. A. Activation of innate immunity by interaction of strains with mutations altering synthesis and assembly of flagellin. B. Release of the FliC180 flagellin into culture supernatants to activate NFκB by HEK cells displaying TLR5.

FIG. 7. Serum antibodies induced to outer membrane and lipo-proteins in mice immunized with S. typhimurium vaccine strains.

FIG. 8. Design of constructions for rhamnose-regulated cessation in expression of waaL gene to alter timing for inability to add LPS O-antigen components onto LPS core.

FIG. 9. Nucleotide sequence for original ΔpagL64::TT rhaRS P_{rhaBAD1} waaL1 deletion-insertion mutation to enable rhamnose-regulated expression of the waaL gene needed for addition of LPS O-antigen to the LPS core (SEQ ID NO 1, 29-31).

FIG. 10. Replacement of ΔpagL64::TT rhaRS P_{rhaBAD1} waaL1 (c12337) with ΔpagL38::TT rhaRS P_{rhaBAD1} waaL2 (c12534) increases the number of cell divisions needed for complete loss of the LPS-O antigen (periodic 1:3 dilution to maintain cell density in log phase) FIG. 11. Evidence that increasing the level of WaaL synthesis increases number of cell divisions needed to decrease/eliminate LPS O-antigen on PIESV cell surface.

FIG. 12. Evidence that inclusion of mutations that eliminate catabolism of arabinose and rhamnose and increase level of WaaL synthesis increase number of cell divisions needed in absence of these sugars to decrease/eliminate LPS O-antigen on PIESV cell surface.

FIG. 13. A. Construction of the virulence plasmid derivative pSTUK201 with the Δ(traM-traX)-36::araC P_{araBAD} lacI TT insertion to enable regulated delayed synthesis of protective antigens and enhance display of biological containment. B. Comparative levels of LacI synthesis in strains with three different chromosomal araC P_{araBAD} lacI insertions giving different levels of LacI depending on arabinose in medium and level of LacI synthesized in strain with new Δ(traM-traX)-36::araC P_{araBAD} lacI TT insertion in place of a 32 kb deletion of tra genes in the virulence plasmid pSTUK201.

FIG. 14. Nucleotide sequence of Δ(traM-traX)-36::araC P_{araBAD} lacI TT insertion-deletion mutation present in pSTUK201 (SEQ ID NO 2, 32).

FIG. 15. The western blot data from an analysis of all the pSTUK araC P_{araBAD} lacI constructs listed in Table 7.

FIG. 16. Levels of LacI synthesis in S. typhimurium strains χ8990, χ9080 and χ9226 in comparison with constructs in the pSTUK plasmid with differing SD sequences.

FIG. 17. DNA sequence information for several isolates from constructs regulating levels of LacI synthesis dependent on SD and start codon sequences.

FIG. 18. Impact of insertion of araC P_{araBAD} lacI construct in pSTUK206 in comparison to wild-type parent on coloniztion ability in internal tissues after oral inoculation into BALB/c mice.

FIG. 19. Regulated delayed lysis vector pG8R256 encoding multiple protective antigens for delivery by multiple T2SSs and lysis.

FIG. 20. Western blot illustration depicting IPTG-dependent synthesis of five protective C. perfringens antigens encoded on pG8R256 in the PIESV strain χ12341.

FIG. 21. Nucleotide and amino acid sequences enabling synthesis of five antigens encoded on pG8R256 with secretion of four by unique T2SSs (SEQ ID NO 3, 33-37, 49-51).

FIG. 22. The AsdA⁺ plasmid pYA3341 (pUC ori) and its derivative pYA4037 (pUC ori) with a Salmonella codon-optimized WHV core with insertion of the influenza virus M2e sequence (19) from which the WHV core sequence was excised, the M2e encoding sequence deleted and replaced with a Salmonella codon-optimized SARS-CoV-2 sequence encoding aa 434 to 508 of the S protein that was then inserted into pYA3341 (pUC ori) to yield the AsdA⁺ plasmid pG8R334 (pUC ori).

FIG. 23. WHVcore-ACE2BD S gene DNA sequences encoding (aa434 to 508) in Asd⁺ vector pG8R334 and Lysis vectors pG8R111 (pBR ori) (to yield pG8R316) and pYA4594 (pUC ori) (to yield pG8R317) (SEQ ID NO 4-5, 38, 52).

FIG. 24. Western blot showing IPTG-dependent WHVCore-ACE2 BD production in χ12615(pG8R334).

FIG. 25. Regulated delayed lysis vectors with Salmonella codon-optimized sequences encoding SARS-CoV-2 Spike protein aa sequence 434-508 in Salmonella codon-optimized WHV core sequence to yield pG8R316 (pBR ori) and pG8R317 (pUC ori) and encoding N protein fused to T3SS to yield pG8R318 (p15A ori).

FIG. 26. SARS-CoV-2 N protein encoding DNA sequences (1260 bp) encoding 429 aa with 47.2% GC content and after codon optimization for high-level expression in Salmonella (51.7% GC content) and addition of C-terminal Dc and 6HisTag sequences (SEQ ID NO 6-7, 39).

FIG. 27. Western blot data indicating IPTG-induced synthesis of SARS-CoV-2 S and N protein antigens in χ12615 containing pG8R316, pG8R317 and pG8R318.

FIG. 28. Western blot data indicating IPTG-induced synthesis of WHVcore-ACE2 BD (Anti-SARS-CoV-2 Spike aa 434-508) in strain c12615(pG8R317) can be detected using mouse neutralized antibody (BEI NR-53796, 1:2,000) and polyclonal rabbit Anti-SARS-CoV-2 Spike Glycoprotein (BEI NR-52947, 1:2,000). 2^{nd} antibody: anti-mouse 1:5,000, anti-rabbit, 1:10,000.

FIG. 29. Western blot data indicating IPTG-induced synthesis of N protein in strain c12615(pG8R318) as detected using monoclonal rabbit antibodies (BEI NR-53791, NR-53793, NR-53794, 1:2,000) and monoclonal mouse antibody (BEI NR-53792, 1:2,000) against the SARS-CoV-2 N protein. 2^{nd} antibody: anti-mouse 1:5,000, anti-rabbit, 1:10,000.

FIG. 30. SARS-CoV-2 S gene sequences for spike protein ACE2BD (aa 434-508) (SEQ ID NO 40) to be inserted into DNA vaccine vector pYA4545 with native sequence (pG8R336) (SEQ ID NO 8), with codon-optimized sequence for expression in Salmonella (pG8R337) (SEQ ID NO 9) and with codon-optimized sequence for expression in humans (pG8R338) (SEQ ID NO 10) for delivery by χ12601.

FIG. 31. DNA sequences encoding SARS-CoV-2 S gene sequences (original GC 37.8%) and after codon optimization for optimal expression in humans (GC 59.2%) for insertion into pYA4545 to yield pG8R339 and pG8R335, respectively (SEQ ID NO 11-12, 41, 53).

FIG. 32. Regulated delayed DNA vaccine vectors containing SARS-CoV-2 S gene sequences for ACE2BD (aa434-508) before (pG8R336) and after codon optimization for expression in Salmonella (pG8R337) and humans (pG8R338) and entire S gene sequence original (pG8R339) and optimized for expression in humans (pG8R335).

FIG. 33. Ab responses against SARS-COV-2 Spike ACE2 BD in mice immunized with χ12615(pG8R316) and χ12615(pG8R317) FIG. 34. IgG, IgG1 and IgG2 antibody responses against the SARS-CoV-2 N protein in mice immunized with χ12615(pG8R110) and χ12615(pG8R318)

FIG. 35. Serum antibody responses against LPS and vaginal mucosal IgA against N protein in mice immunized with χ12615(pG8R110) and χ12615(pG8R318)

FIG. 36. Diagrams of the regulated delayed lysis plasmids pG8R358 (encoding bla SS pspA RX1 and pspA EF5668), pG8R369 (encoding bla SS pspA RX1—EF5668 and plyA), pG8R359 (encoding phtD with a C-terminal T4 foldon and 6-His sequence), pG8R370 (encoding phtD with a C-terminal 6-His sequence), pG8R360 (encoding bla SS phtD with a C-terminal T4 foldon and 6-His sequence), and pG8R371 (encoding bla SS phtD with a C-terminal 6-His sequence).

FIG. 37. Codon-optimized nucleotide sequences for bla SS pspA RX1 and pspA EF5668 gene fusion (SEQ ID NO 13, 42).

FIG. 38. Codon-optimized nucleotide sequence for plyA (SEQ ID NO 14, 43).

FIG. 39. Original and codon-optimized nucleotide sequences for phtD with C-terminal T4 foldon and 6-His sequences (SEQ ID NO 15-16, 44).

FIG. 40. Original and codon-optimized nucleotide sequences for bla SS phtD with C-terminal 6-His sequence (SEQ ID NO 17-18, 45).

FIG. 41. Synthesis of PspA and PlyA antigens in the PIESV strains χ12663 and χ12667 with plasmids pG8R114, pG8R358 and pG8R369 using western blot analyses.

FIG. 42. Synthesis of PhtD antigens in the PIESV strain χ12668 with plasmids pG8R111 and pG8R114 (empty vector controls) and pG8R359, pG8R360, pG8R370 and pG8R371 using western blot analyses.

FIG. 43. (A) Diagram of pG8R231 encoding fusion of B. melitensis gene sequences tf, bp26 and omp31. (B) Sequence of fusion of codon-optimized sequences for the B. melitensis genes tf; bp26 and omp31 in pG8R231 comparison with original sequence (SEQ ID NO 19-20, 46).

FIG. 44. (A) Diagram of pG8R259 encoding fusion of B. melitensis gene sequences bls, lg7/lg12 and Zn/Cu sod. (B) Sequence of fusion of codon-optimized sequences for the B. melitensis genes bls, lg7/lg12 and Zn/Cu sod in pG8R259 comparison with original sequence (SEQ ID NO 21-22, 47).

FIG. 45. Map of the regulated delayed lysis plasmid vector pG8R111 (bottom) used to construct pG8R231 and pG8R259 with red arrows showing the regions of DNA sequence for the DNA primers used to verify the presence of the DNA insert in the recombinant vectors and the resulting gel separation (upper) of the DNA fragments generated by PCR reactions with those primers.

FIG. 46. Growth of χ12688 and χ12702 containing the cloning vector pG8R111 and the two recombinant vectors pG8R231 and pG8R259 in LB broth with 0.1% arabinose and 0.1% rhamnose with and without IPTG.

FIG. 47. Levels of synthesis of the fusion antigens after growth of χ12688 and χ12702 containing pG8R111, pG8R231 and pG8R259 in LB broth with 0.1% arabinose and 0.1% rhamnose with and without IPTG induction.

FIG. 48. Diagram of pYA5293 that has a codon-optimized sequence encoding the Eimeria tenella S07 gene fused to the bla T2SS.

FIG. 49. Diagram of the pG8R316 and pG8R317 derivatives with insertion of the highly conserved sequence encoding the influenza virus M2e in place of sequences encoding the SARS-CoV-2 S gene binding sequences.

FIG. 50. (A) DNA sequences of WSN NP with/without codon optimization (SEQ ID NO 23-24, 48). (B) Diagram of the regulated delayed lysis vectors pYA5245 and pYA5264 encoding the influenza virus WSN NP original and codon optimized sequence.

FIG. 51. Diagram of p15 on plasmid construct for T3SS delivery of the highly conserved influenza virus NP protein fused to two conserved HA T-cell epitope sequences.

FIG. 52. Diagram of the regulated delayed lysis DNA vaccine pYA4859 encoding the influenza virus WSN HA antigen.

### DETAILED DESCRIPTION

The present disclosure builds on previous studies that involved the design of PIESV strains. Problems described in the Background section have been addressed while combining and improving desirable attributes to ensure complete safety and maximize immunogenicity to protect against a diversity of bacterial, viral and parasite pathogens.

This disclosure presents the design, construction and extensive evaluation of significantly improved Protective Immunity Enhanced Salmonella Vaccine (PIESV) vectors for antigen and DNA vaccine delivery with three families of constructed strains to enhance recruitment of innate immunity and maximize induction of protective acquired immunity. The systems are designed to use in protecting various animal and human hosts against infections by bacterial, viral, parasite and fungal pathogens upon identification of the pathogen antigens needed to elicit protective immune responses. The Salmonella Typhimurium vaccine vector strains constructed are derived from the super virulent UK-1 strain that is highly infectious for poultry, swine, ruminants and horses. We have used a vast array of information about Salmonella and all the tools of molecular genetics to design and encode a diversity of regulatory sequences to alter immune suppression, virulence and life processes.

These improved PIESV constructs disclosed herein are designed to benefit from use of the Self-Destructing Attenuated Adjuvant Salmonella (SDAAS) strains (disclosed in: PCT Pub. Curtiss, R. WO 2020/096994) and strains disclosed in WO2021/222696, both of which are incorporated herein by reference) to further enhance induction of long-lasting protective acquired immune responses. Also, PCT pub WO2018/136938 is incorporated herein by reference for background purposes. Improvements described herein include:
A. The elimination of multiple means by which Salmonella (i) suppresses or modulates induction of host immune responses, (ii) produces means to form biofilms and other molecules that enable persistence in vivo or survival if released into the environment and (iii) synthesizes subterfuge antigens to induce non-protective immune responses.
B. Enhancing the recruitment of innate immune responses and tolerance of host natural defenses to more effectively colonize lymphoid tissues.
C. Display of novel improved means for display of regulated delayed attenuation such that the vaccine constructs at time of administration to a vaccinated host to display the same (or better) ability as the wild-type parent to colonize internal effector lymphoid tissues to maximize induction of protective immunity.
D. Enable regulated delayed expression of determinants to augment colonization of internal effector lymphoid tissues.
E. Display of a plasmid-specified regulated delayed synthesis of protective pathogen antigens encoded by codon-optimized sequences to enable the vaccine construct at time of administration to a vaccinated host to display the same (or better) ability as the wild-type parent to colonize internal effector lymphoid tissues to maximize induction of protective immunity.
F. Enhance safety of vaccine constructs by eliminating virulence plasmid encoded determinants for conjugative ability of vaccine cells to potentially deliver genetic information to other bacteria present in the host or environment microbiomes.
G. Display of regulated delayed in vivo lysis to deliver a bolus of synthesized pathogen protective antigen(s) or a DNA vaccine encoding them to further potentiate induction of innate immunity, maximize induction of protective immunity and confer complete biological containment with no vaccine persistence in vivo and no survival if released into the environment.
H. Display attributes in PIESV constructs that enable Differentiation of Infected from Vaccinated Animals (DIVA).
I. Possess attributes that facilitate manufacture of PIESV constructs using fermenter technologies.

### Parental PIESV Vector Strains:

Provided below is a description of the construction and detail of the attributes of three parental PIESV strain families that can be further modified for specific applications.
Family 1: χ12615 ΔP_{murA25}::TT araC P_{araBAD} murA ΔasdA27::TT araC P_{araBAD} c2 ΔwaaL46 ΔpagL38::TT rhaRS P_{rhaBAD2} waaL2 Δ(wza-wcaM)-8 ΔrelA1123 ΔrecF126 ΔsifA26 ΔaraBAD65::TT ΔrhaBADSR515 ΔpagP8 ΔlpxR9 (pSTUK201 Δ(traM-traX)-36:: araC P_{araBAD} lacI TT)
   Two further improved derivatives of χ12615 differing in toxicity of the lipidA endotoxin are:
χ12688 ΔP_{murA25}::TT araC P_{araBAD} murA ΔasdA33 ΔwaaL46 ΔpagL38::TT rhaRS P_{rhaBAD2} waaL2 Δ(wza-wcaM)-8 ΔrelA1123 ΔrecF126 ΔsifA26 ΔaraBAD65::TT ΔrhaBADSR515 ΔpagP8 ΔlpxR9 (pSTUK206 Δ(traM-traX)-41::araC P_{araBAD} lacI TT)
χ12702 ΔP_{murA25}::TT araC P_{araBAD} murA ΔasdA33 Δ(wza-wcaM)-8 ΔrecF12 ΔsifA26 ΔwaaL46 ΔaraBAD65::TT ΔrhaBADSR515 ΔpagL38::TT rhaRS P_{rhaBAD2} waaL2 ΔpagP81::Pₗₚₚ lpxE ΔlpxR9 ΔrelA1123 (pSTUK206 Δ(traM-traX)-41::araC P_{araBAD} lacI TT) (modified to produce MPLA for use in hosts sensitive to lipid A)
Family 2: χ12616 ΔP_{murA25}::TT araC P_{araBAD} murA ΔasdA27::TT araC P_{araBAD} c2 ΔwaaL46 ΔpagL38::TT rhaRS P_{rhaBAD2} waaL2 Δ(wza-wcaM)-8 ΔrelA1123 ΔrecF126 ΔsifA26 P_{mntR44}::TT araC P_{araBAD} mntR ΔP_{fur33}::TT araC P_{araBAD} fur ΔaraBAD65::TT ΔrhaBADSR515 (pSTUK201 Δ(traM-traX)-36:: araC P_{araBAD} lacI TT)
Note: Family 2 is derived from Family 1 and is designed to maximize induction of cross-protective immunity to bacterial species in the family Enterobacteriaceae. The further improved derivative of χ12616 is:
χ12704 ΔP_{murA25}::TT araC P_{araBAD} murA ΔasdA33 ΔwaaL46 Δ(wza-wcaM)-8 ΔrelA1123 ΔrecF126 ΔsifA26 ΔmntR28 ΔP_{fur33}::TT araC P_{araBAD} fur ΔaraBAD65::TT ΔrhaBADSR515 ΔpagL38::TT rhaRS P_{rhaBAD2} waaL2 (pSTUK206 Δ(traM-traX)-41:: araC P_{araBAD} lacI TT)
Family 3: χ12601 ΔP_{murA25}::TT araC P_{araBAD} murA ΔasdA27::TT araC P_{araBAD} c2 ΔwaaL46 ΔpagL38::TT rhaRS P_{rhaBAD1} waaL2 Δ(wza-wcaM)-8 ΔrelA1123 ΔrecF126 ΔsifA26 ΔendA2113 ΔsseL116 ΔtlpA181 (or χ12644 with ΔaraBAD65::TT and ΔrhaBADSR515 added)

The further improved derivative of χ12601 is:
χ12706 ΔP_{murA25}::TT araC P_{araBAD} murA ΔasdA33 ΔwaaL46 ΔpagL64::TT rhaRS P_{rhaBAD1} waaL1 Δ(wza-wcaM)-8 ΔrelA1123 ΔrecF126 ΔsifA26 ΔendA2113 ΔsseL16 ΔtlpA181 ΔrhaBADSR515 ΔaraBAD65::TT

Although not present in the original Family parental strains listed above, the improved derivatives described in this disclosure have or will have ΔeptA4, ΔarnT6, ΔasdA33 or ΔP_{asdA55}::TT araC P_{araBAD} asd (in place of ΔasdA27::TT araC P_{araBAD} c2), ΔompA11, ΔsopB1925, ΔpagP81::Pₗₚₚ lpxE (in place of ΔpagP8), ΔmntR28 (in place of P_{mntR44}::TT araC P_{araBAD} mntR), pSTUK206 Δ(traM-traX)-41:: araC P_{araBAD} lacI TT (in place of pSTUK201 Δ(traM-traX)-36:: araC P_{araBAD} lacI TT), ΔstcABCD, ΔP_{stc53}::P_{murA} stcA53, ΔsafABCD, ΔP_{safA55}::P_{murA} safA55, ΔrecA62, Δalr-3, ΔdadB4, ΔfliC180, ΔfliC2426, ΔfljB217, Δ(hin-fljBA)-209, Δ(agfG-agfC)-999, ΔwaaC41, ΔwaaG42, ΔwbaP45, and mutations to enhance/modify performance.

The genotypic and phenotypic properties of all mutations included in each PIESV or SDAAS strain and how they are added and verified is described further below.

### Plasmid Vectors in PIESV Vector Strain Constructs:

The regulated lysis plasmid vectors that encode synthesis of pathogen protective antigens continue to constitute the plasmid component of the balanced-lethal vector-host system. In addition, a new plasmid vector encoding five different protective antigens, four of which are subject to secretion by four different enhanced Type 2 secretion systems (T2SSs), is described. Also, we have devised a PIESV system with two plasmid vectors, one with low copy number encoding antigen delivery by the T3SS and the other a high copy number DNA vaccine vector encoding an additional protective antigen.

### Definitions

As used herein the specification, “a” or “an” may mean one or more, unless clearly indicated otherwise. As used herein in the claims, when used in conjunction with the word “comprising,” the words “a” or “an” may mean one or more than one.

As used herein, the term “adjuvant” refers to an agent that stimulates and/or enhances an immune response in a subject. An adjuvant can stimulate and/or enhance an immune response in the absence of an antigen and/or can stimulate and/or enhance an immune response in the presence of an antigen. Exemplary embodiments of adjuvants include a live self-destructing attenuated adjuvant Salmonella strain as described in WO/2020/096994A1 and co-pending U.S. provisional App No. 63/017,866, which are incorporated herein in their entirety.

The term “administering” or “administration” of an agent as used herein means providing the agent to a subject using any of the various methods or delivery systems for administering agents or pharmaceutical compositions known to those skilled in the art. Agents described herein may be administered by oral, intradermal, intravenous, intramuscular, intraocular, intranasal, intrapulmonary, intraperitoneal, epidermal, transdermal, subcutaneous, mucosal, or transcutaneous administration.

The term “pathogen” as used herein refers to a bacteria, virus, fungus or parasite that is capable of infecting and/or causing adverse symptoms in a subject.

The term “mutation” refers to a genetic modification of DNA sequences encoding for gene regulation such as promoters (P), protein of RNA gene products and translation and transcription termination (TT) signals. Mutations can be caused by deletions (Δ) and base pair substitutions and by insertions (:: or Ω) into DNA sequences in plasmids or the chromosome. Such mutations cause phenotypic changes in a gene that are independent of the means of gene inactivation. However, to distinguish one mutation from another, “allele: numbers are added after the designated promoter or structural gene in giving the genotype of a particular strain or plasmid. However, for example, the phenotypes of strains with the ΔasdA27, ΔasdA33 and ΔasdA34 mutant alleles are all identical in that all strains require diaminopimelic acid to enable peptidoglycan synthesis and growth.

The term “agent” as used herein refers to either adjuvant and/or vaccine.

The term “attenuated” when referring to a PIESV vector strain or live self-destructing attenuated adjuvant Salmonella (SDAAS) strain refers to a strain that comprises one or more attenuating mutations.

The term “attenuated derivative” refers to a derivative that comprises one or more attenuating mutations.

The term “attenuating mutation” refers to a mutation that reduces infectivity, virulence, toxicity, induction of disease symptoms, and/or impairment of a subject upon administration of a PIESV strain and/or a live SDAAS strain. Examples of attenuating mutations include those mutations that facilitate lysis in vivo (e.g. impairing synthesis of essential constituents of peptidoglycan layer), reduce or impair synthesis of LPS or other cell-surface components, and one or more mutations that provide auxotrophy (e.g. dependence on an amino acid, purine, pyrimidine, or vitamin for growth). Non-limiting example of attenuating mutations include: asd, dap, pur, pyr, thyA, aro, pab, nic, pdx, pmi, galE, murA, fur, cya, crp, phoP, phoQ, slr, dam, recA, alr, dadB, sifA, waaC, wag, ΔwbaP45, and waaL.

The term “regulated delayed attenuation” refers to a construction in which the expression of a gene conferring a virulence attribute is regulated by a sugar-dependent process such that the virulence gene is expressed in the presence of a sugar such as but not limited to arabinose or rhamnose supplied during cultivation of the strain and ceases to be expressed in vivo since the sugar is absent to result is the gradual display of attenuation as a consequence of cell division of the PIESV or SDAAS strain in vivo.

The term “regulated delayed antigen synthesis” refers to a construction in which the expression of a gene specifying synthesis of an antigen is regulated by a sugar-dependent process controlling synthesis of a repressor protein that governs expression of the antigen-encoding gene such that the repressor gene is expressed in the presence of a sugar supplied during cultivation of the strain and ceases to be expressed in vivo since the sugar is absent to result is the gradual increase in the synthesis of the antigen specified by the antigen-encoding gene as a consequence of cell division of the PIESV strain in vivo.

The term “regulated delayed lysis” refers to a construction in which the expression of one or more genes specifying synthesis of peptidoglycan precursors such as but not limited to diaminopimelic acid, D-alanine and muramic acid are regulated by a sugar-dependent process such that the genes are expressed in the presence of a sugar such as but not limited to arabinose or rhamnose supplied during cultivation of the strain and cease to be expressed in vivo since the sugar is absent to result in lysis as a consequence of cell division of the PIESV or SDAAS strain in vivo. The genes conferring the regulated delayed lysis phenotype may be either chromosomal and/or plasmid encoded.

The term “regulated delayed lysis plasmid” refers to a construction in which the expression of one or more genes specifying synthesis of peptidoglycan precursors such as but not limited to diaminopimelic acid, D-alanine and muramic acid that are regulated by a sugar-dependent process are located on a plasmid or DNA vaccine vector encoding synthesis of one or more protective antigens.

A “DNA vaccine vector” encodes antigens to be synthesized in a vaccinated animal host after delivery by a PIESV vector strain.

A “virulence plasmid” is present in several serotypes of S. enterica including S. typhimurium and encodes genes that enable these strains of Salmonella to be invasive into internal organ tissues of an infected subject and also genes that enable the conjugational self-transfer to other bacterial strains (i.e., by horizontal gene transfer) by cell-cell contact.

The term “biological containment” refers to a PIESV or SDAAS strain that undergoes regulated delayed lysis in vivo such that the strain cannot persist in vivo or survive if shed into the environment.

The term “biologically contained plasmid” refers to a plasmid that lacks genetic information to enable its conjugational transfer to another bacterial cell.

As used herein, “codon” means, interchangeably, (i) a triplet of ribonucleotides in an mRNA which is translated into an amino acid in a polypeptide or a code for initiation or termination of translation, or (ii) a triplet of deoxyribonucleotides in a gene whose complementary triplet is transcribed into a triplet of ribonucleotides in an mRNA which, in turn, is translated into an amino acid in a polypeptide or a code for initiation or termination or translation or a tRNA to act as a carrier of an amino acid to be incorporated into a growing amino acid chain as specified by an mRNA. Thus, for example, 5′-TCC-3′ and 5′-UCC-3′ are both “codons” for serine, as the term “codon” is used herein.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps.

The term “consisting essentially of” when used in conjunction with PIESV containing compositions described herein refers to a composition comprising a PIESV and a pharmaceutical carrier and/or an adjuvant without any other immune response enhancing components.

The term “derivative” with respect to a Salmonella strain or Protective Immunity Enhanced Salmonella Vaccine (PIESV) vector strain, refers to descendant cells thereof that include a genetic modification that include deletion, replacement/substitution and/or addition mutations.

The term “co-administration” or “co-administering” as used herein refers to the administration of an active agent before, concurrently, or after the administration of another active agent such that the biological effects of either agents overlap.

The term “gene”, as used herein, refers to a nucleic acid sequence that encodes for the synthesis of a specific protein. In some embodiments, a gene may include a regulatory sequence of a 5′-non-coding sequence and/or a 3′-non-coding sequence or may encode for synthesis of a functional RNA such as tRNA or rRNA playing some role in cellular function.

The term “descendant(s)” refers to cells resulting from cell division of a Salmonella cell.

The term “genetic modification” as used herein refers to removal, alteration, replacement or addition of a gene in a Salmonella cell.

The term “subject” or “host” refers to an individual susceptible to infection by a pathogen or responsive to administration of a vaccine to prevent or ameliorate consequences of infection by a pathogen.

An “immune response enhancing amount” is that amount of an adjuvant administered sufficient to enhance an immune response of vaccine administration in a subject compared to vaccine administration without adjuvant administration. An immune response enhancing amount can be administered in one or more administrations.

As used herein, the term “immunogen” refers to an antigen that is recognized as unwanted, undesired, and/or foreign in a subject.

A used herein, the term “immune response” includes a response by a subject's immune system to a vaccine. Immune responses include both cell-mediated immune responses (responses mediated by antigen-specific T cells and non-specific cells of the immune system) and humoral immune responses (responses mediated by antibodies present in the plasma lymph, and tissue fluids). The term “immune response” encompasses both the initial responses to an immunogen as well as memory responses that are a result of “acquired immunity.”

The term “live self-destructing attenuated adjuvant Salmonella (SDAAS) strain” refers to a Salmonella strain that possesses one or more attenuating mutations some of which specify lysis in vivo and is useful as an adjuvant.

As used herein, “nucleic acid” or “nucleic acid sequence” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., a-enantiomeric forms of naturally-occurring nucleotides), or a combination of both and can be either single- or double-stranded.

The term “fragment” as used herein refers to a small part of the polynucleotide or polypeptide sequences of the disclosure. Representative fragments of the polynucleotide or polypeptide sequences according to the disclosure will be understood to mean any nucleotide or peptide fragment having at least 5 successive nucleotides or peptides, preferably at least 12 successive nucleotides or peptides, and still more preferably at least 15, 18, or at least 20 successive nucleotides or peptides of the sequence from which it is derived. The upper limit for such fragments is the total number of nucleotides or peptides found in the full-length sequence encoding a particular polypeptide (e.g., a polypeptide such as that of SEQ ID NO: 33). The term “successive” can be interchanged with the term “consecutive” or the phrase “contiguous span”. Thus, in some embodiments, a polynucleotide or polypeptide fragment may be referred to as “a contiguous span of at least X nucleotides or peptides, wherein X is any integer value beginning with 5; the upper limit for such fragments is one nucleotide or peptide less than the total number of nucleotides or polypeptides found in the full-length sequence encoding a particular polypeptide (e.g., a polypeptide comprising SEQ ID NO: 33).

The term “pathogenic Salmonella” as used herein refers to a bacterium of the Salmonella genera {e.g., S. enterica or S. bongori). Pathogenic Salmonella may include species or serovar (subspecies) of the Salmonella genera. For example, pathogenic Salmonella may be a Salmonella enterica serovar, including, for example, Paratyphi A, Enteritidis, Typhi, and Typhimurium. In some embodiments, the recombinant bacterium is of the serovar S. typhimurium, S. typhi, S. paratyphi, S. gallinarum, S. enteritidis, S. choleraesius, S. arizonae, S. newport, S. heidelberg, S. infantis, S. cholerasiuis, or S. dublin.

The term “pharmaceutically acceptable carrier” as used herein refers to one or more formulation materials suitable for accomplishing or enhancing the successful delivery of the pharmaceutical composition of the PIESV disclosed herein. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990). Examples of liquid carriers include, but are not limited to, water, saline, dextrose, glycerol, ethanol and mixtures thereof.

The term “programmed” as used herein refers to engineering of a plasmid vector or derivative so as to be constructed to effect a noted result such as regulated delayed antigen synthesis, regulated delayed lysis, etc.

The term “Protective Immunity Enhanced Salmonella Vaccine” or “(PIESV)” refers to a PIESV vector strain that has been engineered to synthesize and deliver an immunogen or a DNA vaccine encoding an immunogen.

The term “Protective Immunity Enhanced Salmonella Vaccine vector strain” or “PIESV vector strain” refers to a strain of Salmonella that has one or more attenuating mutations and is capable of being engineered to synthesize or deliver an immunogen or a sequence encoding an immunogen.

The term “protective immunity” as used herein refers to induction of an immune response upon administration of a vaccine sufficient to confer protection against a pathogen.

The term “recombinant” as used herein refers to a protein, nucleic acid construct, derivative, or cell, generated recombinantly or synthetically, e.g., in the case of a protein, through the translation of the RNA transcript of a particular vector or plasmid-associated series of specified nucleic acid sequence or of an expression cassette in a host bacterial cell. The term “recombinant” as used herein does not encompass the alteration of the cell or vector by naturally occurring events (e.g., spontaneous mutation, natural transformation/transduction/transposition) such as those occurring without deliberate human intervention.

As used herein, the phrase “stimulating or enhancing an immune response” refers to an increase in an immune response in the subject following administration of a vaccine with an adjuvant of the disclosed embodiments relative to the level of immune response in the subject when a vaccine has been administered without an adjuvant.

The term “subject” as used herein refers to a human or non-human animal. Non-human animals include but are not limited to cows, pigs, horses, dogs, cats, camels, alpaca, sheep, goats, birds and reptiles. (Snakes, turtles and other reptiles are susceptible to Salmonella and can potentially be vaccinated with PIESV constructs.)

As used herein, the term “vaccine” refers to an immunogen or a composition comprising an immunogen that elicits an endogenous immune response in a subject (e.g., a human or animal). The endogenous immune response may result in, for example, the switching of a Th1 biased immune response to a Th2 biased immune response, the activation or enhancement of T effector cell responses and/or the reduction of T regulatory cell response, the activation of antigen-specific naive lymphocytes that may then give rise to antibody-secreting B cells or antigen-specific effector and memory T cells or both, and/or the direct activation of antibody-secreting B cells. Typically, a vaccine provides for protective immunity against a pathogen.

In addition to all the components of the immune system described above, infection of a host by pathogens can trigger responses of the nervous (29, 30) and endocrine (31, 32) systems that can contribute to what is collectively referred to by the term “protective immunity” as a consequence of vaccination of a host with a PIESV whether augmented by a SDAAS or not.

### GENERAL DESCRIPTION OF EXEMPLARY EMBODIMENTS

According to certain embodiments, provided is an attenuated derivative of a pathogenic Salmonella that comprises one or more genotypic/phenotypic properties of χ12688 and χ12702, or descendants or derivatives thereof. In a specific example, the attenuated derivative includes one or more of the following genetic modifications: ΔP_{murA25}::TT araC P_{araBAD} murA, ΔasdA33, ΔwaaL46, ΔpagL38::TT rhaRS P_{rhaBAD2} waaL2, Δ(wza-wcaM)-8, ΔrelA1123, ΔrecF126, ΔsifA26, ΔaraBAD65::TT, ΔrhaBADSR515, ΔpagP8 or ΔpagP81::Pₗₚₚ lpxE, ΔlpxR9, (pSTUK206 or Δ(traM-traX)-41:: araC P_{araBAD} lacI TT). In a specific example, the attenuated derivative includes all of the genetic modifications in the preceding sentence. In another embodiment, the attenuated derivative includes one or more of the following genetic modifications: ΔeptA4, ΔarnT6, ΔP_{asdA55}::TT araC P_{araBAD} asd or ΔP_{asdA77}::TT P_{rhaBAD1} asd or ΔP_{asdA88}::TT rhaRS P_{rhaBAD1} asd (in place of ΔasdA33), ΔompA11, ΔsopB1925, ΔpagP81::Pₗₚₚ lpxE (in place of ΔpagP8), ΔstcABCD, ΔP_{stc53}::P_{murA} stcA53, ΔsafABCD, ΔP_{safA55}::P_{murA} safA55, ΔrecA62, Δalr-3, ΔdadB4 or ΔP_{dadB66}::TT araC P_{araBAD} dadB or ΔP_{dadB99}::TT P_{rhaBAD} dadB, ΔfliC180, ΔfliC2426, ΔfljB217, Δ(hin-fljBA)-209, Δ(agfG-agfC)-999, ΔwaaC41, ΔwaaG42, ΔwbaP45, ΔpagL64::TT araC P_{araBAD1} waaL and Δ(traM-traX)-36::araC P_{araBAD} lacI TT or Δ(traM-traX)-37::araC P_{araBAD} lacI TT or Δ(traM-traX)-38::araC P_{araBAD} lacI TT or Δ(traM-traX)-39::araC P_{araBAD} lacI TT or Δ(traM-traX)-40::araC P_{araBAD} lacI TT (in place of Δ(traM-traX)-41::araC P_{araBAD} lacI TT) mutations. In a specific embodiment, the attenuated derivative includes all of the mutations of the preceding sentence, and, optionally, those enumerated in this paragraph above the preceding sentence.

In a certain embodiment, the attenuated derivative of a pathogenic Salmonella is a recombinant attenuated derivative as described above that further comprises a nucleic acid sequence encoding an immunogen. In a specific embodiment, the recombinant attenuated derivative of a pathogenic Salmonella synthesizes and delivers the immunogen when inoculated into a subject. In specific examples, the immunogen encoded by the nucleic acid sequence is selected from the group consisting of PspA, PlyA, PhtD, tf, Bp26, Omp31, Bls, lg7/lg12, or Zn/Cu SOD

According to another embodiment, provided is an attenuated derivative of a pathogenic Salmonella that comprises the genotypic/phenotypic properties of χ12704, descendants or derivatives thereof. In a specific embodiment, the attenuated derivative includes one or more of the following genetic modifications: ΔP_{murA25}::TT araC P_{araBAD} murA, ΔasdA33, ΔwaaL46, Δ(wza-wcaM)-8, ΔrelA1123, ΔrecF126, ΔsifA26, ΔmntR28, ΔP_{fur33}::TT araC P_{araBAD} fur, ΔaraBAD65::TT, ΔrhaBADSR515, ΔpagL38::TT rhaRS P_{rhaBAD2} waaL2, (pSTUK206 Δ(traM-traX)-41:: araC P_{araBAD} lacI TT). In a specific embodiment, the attenuated derivative includes all of the genetic modifications set forth in the preceding sentence. In another embodiment, the attenuated derivative includes one or more of the following genetic modifications: ΔeptA4, ΔarnT6, ΔP_{asdA55}::TT araC P_{araBAD} asd or ΔP_{asdA77}::TT P_{rhaBAD1} asd or ΔP_{asdA88}::TT rhaRS P_{rhaBAD1} asd (in place of ΔasdA27::TT araC P_{araBAD} c2), ΔompA11, ΔsopB1925, ΔpagP81::Pₗₚₚ lpxE (in place of ΔpagP8), ΔstcABCD, ΔP_{stcs3}::P_{murA} stcA53, ΔsafABCD, ΔP_{safA55}::P_{murA} safA55, ΔrecA62, Δalr-3, ΔdadB4 or ΔP_{dadB66}::TT araC P_{araBAD} dadB or ΔP_{dadB99}::TT P_{rhaBAD} dadB, P_{mntR44}::TT araC P_{araBAD} mntR, ΔfliC180, ΔfliC2426, ΔfljB217, Δ(hin-fljBA)-209, Δ(agfG-agfC)-999, ΔwaaC41, ΔwaaG42, ΔwbaP45, ΔpagL64::TT araC P_{araBAD1} waaL and Δ(traM-traX)-36::araC P_{araBAD} lacI TT or Δ(traM-traX)-37::araC P_{araBAD} lacI TT or Δ(traM-traX)-38::araC P_{araBAD} lacI TT or Δ(traM-traX)-39::araC P_{araBAD} lacI TT or Δ(traM-traX)-40::araC P_{araBAD} lacI TT (in place of Δ(traM-traX)-41::araC P_{araBAD} lacI TT) mutations. In a specific embodiment, the attenuated derivative includes all of the genetic modifications of the preceding sentence, and, optionally, those of the specified in the paragraph above the preceding sentence.

In a specific embodiment, the attenuated derivative of a pathogenic Salmonella having the genotypic/phenotypic properties of χ12704a is a recombinant attenuated derivative that further comprises a nucleic acid sequence encoding an immunogen, and wherein the recombinant attenuated derivative synthesizes and delivers the immunogen when inoculated into a subject. The immunogen pertain to an example such as an immunogen selected from the group consisting of S07, BlaSS PlcC, GST-NetB, PelBSS Fba, DsbASS Cbh-6HisTag, OmpASS CpeCMax-6HisTag, M2e,

According to a further embodiment, provided is an attenuated derivative of a pathogenic Salmonella that comprises the genotypic/phenotypic properties of χ12706, or descendants or derivatives thereof. The attenuated derivative of a pathogenic Salmonella includes one or more of the following genetic modifications: ΔP_{murA25}::TT araC P_{araBAD} murA, ΔasdA33, ΔwaaL46, ΔpagL38::TT rhaRS P_{rhaBAD2} waaL2, Δ(wza-wcaM)-8. ΔrelA1123, ΔrecF126, ΔsifA26, ΔendA2113, ΔsseL16, ΔtlpA18, ΔrhaBADSR515, ΔaraBAD65::TT. In a specific example, the attenuated derivative includes all of the genetic modifications enumerated in the preceding sentence. In another example the attenuated derivative of a pathogenic Salmonella includes one or more of the following genetic modifications: ΔeptA4, ΔarnT6, ΔP_{asdA55}::TT araC P_{araBAD} asd or ΔP_{asdA77}::TT P_{rhaBAD1} asd or ΔP_{asdA88}::TT rhaRS P_{rhaBAD1} asd or ΔasdA27::TT araC P_{araBAD} c2 (in place of ΔasdA33), ΔompA11, ΔsopB1925, ΔpagP81::Pₗₚₚ lpxE (in place of ΔpagP8), ΔstcABCD, ΔP_{stc53}::P_{murA} stcA53, ΔsafABCD, ΔP_{safA55}::P_{murA} safA55, ΔrecA62, Δalr-3, ΔdadB4 or ΔP_{dadB66}::TT araC P_{araBAD} dadB or ΔP_{dadB99}::TT P_{rhaBAD} dadB, ΔfliC180, ΔfliC2426, ΔfljB217, Δ(hin-fljBA)-209, Δ(agfG-agfC)-999, ΔwaaC41, ΔwaaG42, ΔwbaP45, and ΔpagL64::TT araC P_{araBAD1} waaL mutations. The attenuated derivative may include all of the mutations in the preceding sentence, and, optionally, those of the specified in the paragraph above the preceding sentence. In a specific example, the attenuated derivative of a pathogenic Salmonella is a recombinant attenuated derivative that further comprises a DNA vaccine vector with a nucleic acid sequence encoding an immunogen, and wherein the recombinant attenuated derivative delivers the DNA vaccine to a subject to be expressed in said subject. In an even more specific embodiment, the immunogen comprises WSN HA.

According to another embodiment, provided is a modified biologically contained Salmonella virulence plasmid with deletions of DNA sequences that eliminate the potential for conjugational transfer and encode synthesis of a regulatory protein dependent on the presence of a metabolizable sugar that is absent in subject tissues.

In a further embodiment, disclosed is an attenuated derivative of a pathogenic Salmonella in which display of genotypic/phenotypic properties are dependent on the presence of two or more metabolizable sugars that are unable to be catabolized by the Salmonella and that are absent in subject tissues.

In yet another embodiment, provided is an attenuated derivative of a pathogenic Salmonella comprising genes encoding Fur and MntR whose expression is dependent on the presence of metabolizable sugars that are absent in animal tissues to result in high-level expression in vivo of means for acquisition of iron and manganese.

Another embodiment disclosed herein pertains to an attenuated derivative of a pathogenic Salmonella comprising genes encoding the Stc and Saf fimbriae that are constitutively expressed in vivo to augment colonization of spleens in an inoculated subject.

A further embodiment pertains to a programmed regulated delayed lysis in vivo plasmid vector encoding for synthesis of four or more heterologous protein antigens with three or more antigens secreted by different type 2 secretion systems. In a specific example, the plasmid vector contains a nucleic acid sequence comprising SEQ ID NO: 3. Further, the protein antigens may be selected from the group consisting of the amino acid sequences of any of SEQ ID NOs: 33-37, SEQ ID NO 42, or SEQ ID NOs: 44-51 or a fragment thereof of at least 10, 20, 30, 40, or 50 contiguous amino acids.

In yet another embodiment, provided is a recombinant attenuated derivative of a pathogenic Salmonella programmed for regulated delayed antigen synthesis and release by regulated delayed lysis of a WHV core fused to a SARS-CoV-2 segment of the S protein specifying an ACE2 binding domain. In a specific example, the ACE2 binding domain comprises an amino acid sequence of SEQ ID NO: 40 or a fragment thereof of at least 10, 20, 30, 40, or 50 contiguous amino acids. In another specific example, the WHV core fused to the SARS-CoV-2 segment of the S protein specifying the ACE2 binding domain comprises an amino acid sequence of SEQ ID NO: 38 or SEQ ID NO: 52 or a fragment thereof of at least 10, 20, 30, 40, or 50 contiguous amino acids.

According to another embodiment, provided is a recombinant attenuated derivative of a pathogenic Salmonella programmed for regulated delayed antigen synthesis of a SARS-CoV-2 protein antigen fused to a type 3 secretion system effector to be delivered by both type 3 secretion and by regulated delayed lysis in an inoculated subject. In a specific example, the SARS-CoV-2 protein antigen comprises an amino acid sequence of SEQ ID NO: 39, SEQ ID NO: 41, SEQ ID NO: 53 or a fragment thereof of at least 10, 20, 30, 40, or 50 contiguous amino acids.

Another embodiment pertains to a recombinant attenuated derivative of a pathogenic Salmonella programmed for regulated delayed lysis to release a DNA vaccine that expresses one or more proteins encoded by a SARS-CoV-2 genome, wherein the DNA vaccine comprises at least one nucleic acid sequence of the SARS-CoV-2 genome, and wherein the at least one nucleic acid sequence comprises one or more optimized codons designed to enhance expression in the inoculated subject. In a specific example, the at least one nucleic acid sequence is selected from the group consisting of nucleic acid sequences of SEQ ID NOs: 4-12 or a fragment thereof of at least 10, 20, 30, 40, or 50 contiguous nucleic acids.

Further embodiments pertain to attenuated derivatives described herein that are in a composition comprising a pharmaceutically acceptable carrier.

Other embodiments pertain to a method of inducing an immune response comprising administering to a subject at least one or more attenuated derivative as described herein, wherein the immune response is against an immunogen produced by the attenuated derivative. In specific examples, administering provides protective immunity against S. pneumoniae, B. melitensis, Eimeria, C. perfringens, or avian influenza virus. In another specific embodiment, administering provides protective immunity against SARS-CoV-2.

### Sequence Variability

In various embodiments, the present disclosure comprises a range of polynucleotide and polypeptide sequences directed to PIESV vectors that encode immunogens. Those skilled in the art will appreciate that specific sequences described herein and provided in the drawings may have sequence variability and still achieve the intended biological function or outcome. Acceptable variability includes approximately 80% to 100% sequence identity and any integer value therebetween. Typically sequence identity is at least 70%, at least 75%, at least 80% at least 85 percent or preferably at least 90% more preferable at least 95% or at least 98% to the referenced sequence.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. 0. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six).

From the data generated, the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

### Compositions and Modes of Administration

In various embodiments, the methods of the present disclosure comprises administering an attenuated derivative of a pathogenic Salmonella or PIESV vectors that encode at least one antigen. In a specific embodiment, the methods are directed to administering or co-administering live self-destructing attenuated adjuvant Salmonella strains. Administration of the vector and/or one or more adjuvant components can comprise, for example, inhalative, intramuscular, intravenous, peritoneal, subcutaneous, and intradermal administration.

In a specific example, the vector and/or adjuvant component(s) are administered concurrently. In an even more specific example, the vector and adjuvant component(s) are administered in the same composition.

Furthermore, the actual dose and schedule can vary depending on whether the vector and adjuvant component(s) are administered in combination with other pharmaceutical compositions, or depending on inter-individual differences in pharmacokinetics, drug disposition, and metabolism. One skilled in the art can easily make any necessary adjustments in accordance with the exigencies of the particular situation.

These methods described herein are by no means all-inclusive, and further methods to suit the specific application will be apparent to the ordinary skilled artisan. Moreover, the effective amount of the vector and/or adjuvant component(s) can be further approximated through analogy to compounds known to exert the desired effect.

Administration of the vector and/or adjuvant component(s) may be continuous or intermittent, depending, for example, upon the recipient's physiological condition, whether the purpose of the administration is therapeutic or prophylactic, and other factors known to skilled practitioners. The administration of the vector and/or adjuvant component(s) may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

When the vector and/or adjuvant component(s) are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A“pharmaceutically acceptable” carrier, diluent, excipient, and/or salt is one that is compatible with the other ingredients of the formulation, and not deleterious to the recipient thereof. The active ingredient for administration may be present as a powder, as granules, as a solution, as a suspension or as an emulsion.

Pharmaceutical formulations containing the vector and/or adjuvant component(s) can be prepared by procedures known in the art using well known and readily available ingredients. The vector and adjuvant component(s) can also be formulated as solutions appropriate for inhalative administration or parenteral administration, for instance by intramuscular, subcutaneous, intradermal or intravenous routes.

The pharmaceutical formulations of vector and/or adjuvant component(s) can also take the form of an aqueous or anhydrous solution or dispersion, or alternatively the form of an emulsion or suspension.

Thus, for parenteral administration (e.g., by injection, for example, bolus injection or continuous infusion) the formulations may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

Devices for intranasal administration include spray devices. Suitable nasal spray devices are commercially available from Becton Dickinson, Pfeiffer GMBH and Valois. Spray devices for intranasal use do not depend for their performance on the pressure applied by the user. Pressure threshold devices are particularly useful since liquid is released from the nozzle only when a threshold pressure is attained. These devices make it easier to achieve a spray with a regular droplet size. Pressure threshold devices suitable for use with the present invention are known in the art and are described for example in WO 91/13281 and EP 311 863 B. Such devices are currently available from Pfeiffer GmbH and are also described in Bommer, R.

### EXAMPLES

### Example 1. Materials and Methods

a. Bacterial Strains, Media and Bacterial Growth.

All SDAAS and PIESV strains are derived from the highly virulent S. typhimurium UK-1 strain (33) since attenuated S. typhimurium UK-1 strains will induce protective immunity to challenge with all S. typhimurium strains whereas other S. typhimurium strains attenuated with the same mutations often cannot induce protective immunity to some S. typhimurium strains and definitely not to virulent UK-1 (34, 35). LB broth and agar (36) and Un-Purple broth (PB) (Difco), which is devoid of arabinose (Ara), mannose (Man) and rhamnose (Rha), are used as complex media for propagation, phenotypic analyses and plating. MacConkey agar with 0.5% lactose (Lac) and 0.1% Ara are used to enumerate bacteria recovered from mice or other animals. Bacterial growth is monitored spectrophotometrically and by plating for colony counts. All studies with PIESV and SDAAS strains with and without plasmid vectors encoding antigens are done in conformity with guidelines established by the UF EH&S and approved by the UF IBC.

b. Molecular and Genetic Procedures.

Methods for DNA isolation, restriction enzyme digestion, DNA cloning and use of PCR for construction and verification of bacterial strains and vectors are standard (37). DNA sequence analyses are performed commercially. All oligonucleotide and/or gene syntheses are done commercially with codon optimization to enhance translational efficiency in humans or Salmonella and stabilize mRNA to “destroy” RNase E cleavage sites (38, 39) to prolong mRNA half-life. Plasmids are evaluated by DNA sequencing and ability to specify synthesis of proteins using gel electrophoresis and western blot analyses. Expression of sequences encoded in DNA vaccine vectors is monitored after electroporation into Vero cells and using antibodies specific to DNA vaccine encoded proteins.

Methods for generating mutant strains are described in previous publications (40-48) and in Examples below. Recombinant plasmid constructs are transformed into E. coli χ6212 (ΔasdA4) with selection for AsdA⁺ for initial characterization prior to electroporation into PIESV strains. χ6212 with the pYA232 or pYA812 plasmid encoding the lac^{q} sequence is often used to cause expression of P_{trc} regulated sequences encoded on constructed plasmids to be dependent on addition of IPTG.

c. Protective Antigen Selection.

Selection of protective antigens to be encoded on regulated lysis plasmid vectors for synthesis and delivery by PIESV strains or to be encoded on DNA vaccine vectors for expression in the vaccinated animal host are based on evidence in the published literature or deduced based on data for contribution to virulence or elicitation of protective immune responses or based on bioinformatic searches using known information about other pathogens.

d. PIESV Strain Characterization.

PIESV constructs are evaluated in comparison with empty vector-control strains for stability of plasmid maintenance, integrity and protein synthesis ability when PIESVs are grown in the presence of arabinose and DAP and with and without IPTG for 50 generations. The IPTG dependence of protein synthesis to overcome the LacI repression of the P_{trc} promoter is also verified. IPTG-induced cultures are incubated with chloramphenicol to arrest protein synthesis to determine whether plasmid-specified proteins are stable during the next 4 h. If not, the nucleotide sequence is altered to eliminate protease cleavage sites (with subsequent comparison of both constructs for induction of immune responses). Measurement of LPS core and O-antigen is performed after electrophoresis using silver-stained gels (49). Final PIESV constructs are evaluated for bile sensitivity, acid tolerance and ability to survive in sera with and without complement (46-48) and for sensitivity to antibiotics used to treat Salmonella infections.

e. Cell Culture Methods and Use of HEK293 Cells to Monitor Initiation of Innate Immune Responses.

HEK293 cells with the murine TLR2, TLR4, TLR5, TLR7, TLR8, TLR9, NOD1 and NOD2 with the NF-kB SEAP reporter system to enable read outs at A650 nm (50) are used to evaluate activation of innate immune responses. We grow SDAAS and PIESV strains to maximize cell invasiveness and then determine their attachment to, invasion into and survival in Int-407, RAW264.7 and HEK cells. NF-kB production using various MOIs of SDAAS and PIESV cells to HEK cells are measured over a 24 h period (50).

f. Animal Experimentation to Monitor Safety and Efficacy of SDAAS Strains and Determine their Ability to Augment Level of Protective Immunity by PIESV Constructs.

Adult BALB/c mice (6- to 8-weeks old) of both sexes are used. They are allowed to acclimate in the animal facilities for one week prior to use. PIESV and SDAAS strains are grown in LB broth with necessary supplements to an OD₆₀₀ of ˜0.9, sedimented by centrifugation at room temperature and suspended in PBS at densities of 5×10¹⁰ CFU/ml to enable i.n. and oral doses of up to 1×10⁹ CFU to be administered in 20 μl per mouse. SDAAS and PIESV strains are administered at different doses by different routes as described in the Examples. In some studies, fresh fecal pellets are collected to measure excretion of viable PIESV and SDAAS strains, if any. Sera and mucosal fluids are collected for quantitation of specific IgG and SIgA antibodies at two-week intervals. In some studies, sufficient numbers of mice are immunized to collect sera to conduct studies on neutralization of viruses such as SARS-CoV-2 and influenza virus infection into virus-susceptible cells. In other studies, intracellular IFN-γ increases are measured in peripheral blood TCRβ⁺ CD4⁺, CD8⁺ and CD17⁺ T cells harvested in gradient-isolated mononuclear cells at different times after immunization. Studies on safety of PIESV and SDAAS strains sometimes use pregnant, newborn, malnourished and immunocompromised mice. All experimental work is conducted in compliance with the regulations and policies of the Animal Welfare Act and the Public Health Service Policy on Humane Care and Use of Laboratory Animals and approved by the University of Florida IACUC.

g. Monitoring Immune Responses.

i. Antigen Preparation: Salmonella B group LPS 0-antigen is obtained commercially. A S. typhimurium outer membrane protein (SOMP) fraction (isolated from a mutant strain that is deficient in making LPS O-antigen and outer core, in vitro synthesized fimbrial antigens and flagella) has been prepared. Purified antigens representing all those used in PIESV strains previously described (11, 20) are available, often purified using C-terminal His tags and nickel column technology. These antigens are used for immunoassays as described below. ii. ELISA: Serum antibodies are measured in blood collected by submandibular bleeding. To distinguish between Th1 and Th2 responses, titers of IgG1 and IgG2a are determined. The 96-well plates are coated with 100 ng antigen. Free binding sites are blocked with the SEABLOCK Blocking Buffer (Thermo Fisher). Antibody titers in sera (diluted 1:100) and secretions (diluted 1:10) are detected with biotinylated goat anti-mouse IgG, IgG1, IgG2a or IgA (Southern Biotechnology) followed by incubation with a streptavidin-alkaline phosphatase conjugate (Southern Biotechnology). Color development (absorbance at 405 nm) with p-nitrophenyl phosphate (Thermo Fisher Scientific) is recorded with an automated ELISA plate reader (EL311SX; Biotek). Unconjugated mouse antibodies (Southern Biotechnology) (IgG, 5 μg/ml to 40 ng/ml; IgG1 and IgG2a, 1 μg/ml to 8 ng/ml; IgA 62.5 ng/ml to 0.46 ng/ml) are serially diluted and coated on a 96-well plate in duplicate. Standard curves are generated by plotting the OD₄₀₅ values against the representative concentrations of the diluted unconjugated antibody solutions and fitted to a 4-parameter logistic curve (R2≥0.98). The absorbance values of experimental samples are fit into the standard curve to interpolate antibody concentrations. All samples are analyzed in triplicate. iii. Cellular immune responses and Flow cytometry analyses: Flow cytometry is used to evaluate induction of antigen-specific CD4, CD8 and CD17 responses and induction of antigen-dependent cytokine responses (51-57). iv. Monitoring cell concentrations in collected whole blood: Concentrations of lymphocytes, monocytes, NK cells, DCs, macrophages, etc. are determined using standard analytical methods.

h. Statistical Analyses and Scientific Rigor.

All studies are repeated and results independently corroborated. Results are analyzed using the most appropriate statistical test from the SAS program to evaluate the relative significance or lack thereof of results obtained. In specific cases, staff at the Clinical Translational Science Institute at UFL have been consulted to provide help with experimental design and statistical analyses services for animal and human studies and trials.

### Example 2. Design and Construction of Deletion and Deletion-Insertion Mutations and Introduction into S. typhimurium UK-1 χ3761

Mutant alleles were designed by using the GenBank DNA information to identify sequences encoding the gene product of interest and flanking sequences to generate suicide vectors containing just the flanking sequences adjacent to the sequence to be deleted. The deleted sequence might be the entire open reading frame from start to stop codons inclusive or only a promoter region with upstream sequences or a portion of a gene sequence to be deleted or substituted with a modification and for deletion-insertion mutations with an inserted sequence to substitute for a deleted sequence. We have used the suicide vectors pRE112 and pMEG375 and used either conjugational transfer of the constructed suicide vector into target strains using the suicide vector donor strain χ7213 or by the transductional method (42) with selection of suicide vector integration followed by selective excision. These methods are applicable for modification of chromosomal or plasmid genes. Table 1 lists all the suicide vectors used for each gene deletion or modification and Table 2 lists all the mutations and their associated phenotypes.

TABLE 1
Suicide vectors for constructing the mutations listed in Table 2

| | | |
|---|---|---|
| Genotype | Suicide Vector | Marker |

A. Deletion and deletion-insertion mutations to facilitate regulated delayed lysis in vivo

| | | |
|---|---|---|
| ΔP_{murA25}::TT araC P_{araBAD} murA | pYA4686 | Cm |
| ΔasdA33 | pYA3736 | Cm |
| ΔP_{asdA55}::TT araC P_{araBAD} asdA | pG8R71 | Cm |
| ΔP_{asdA77}::TT P_{rhaBAD1} asd | pG8R355 | Cm |
| ΔP_{asda88}::TT rhaRS P_{rhaBAD1} asd | pG8R354 | Cm |
| Δalr-3 | pYA3667 | Cm |
| ΔdadB4 | pYA3668 | Cm |
| ΔP_{dadB66}::TT araC P_{araBAD} dadB | pG8R73 | Cm |
| ΔP_{dadB99}::TT P_{rhaBAD} dadB | pG8R353 | Cm |
| Δ(wza-wcaM)-8 | pYA4368 | Cm |
| ΔrelA1123 | pYA3679 | Cm |

B. Mutations enabling regulation of genes that might be present on plasmid vectors in
conjunction with strains undergoing regulated delayed lysis in vivo

| | | |
|---|---|---|
| ΔrelA197::araC P_{araBAD} lacI TT | pYA4064 | Cm |
| ΔasdA27::TT araC P_{araBAD} c2 | pYA4138 | Cm |
| Δ(traM-traX)-36::araC P_{araBAD} lacI TT | pG8R329 | Cm |
| Δ(traM-traX)-37::araC P_{araBAD} lacI TT | pG8R377 | Cm |
| Δ(traM-traX)-38::araC P_{araBAD} lacI TT | pG8R378 | Cm |
| Δ(traM-traX)-39::araC P_{araBAD} lacI TT | pG8R379 | Cm |
| Δ(traM-traX)-40::araC P_{araBAD} lacI | pG8R380 | Cm |
| Δ(traM-traX)-41::araC P_{araBAD} lacI TT | pG8R381 | Cm |

C. Mutations conferring regulated delayed attenuation and over production of iron and
manganese-regulated proteins to confer cross-protective immunity

| | | |
|---|---|---|
| ΔP_{fur33}::TT araC P_{araBAD} fur | pYA3722 | Cm |
| ΔP_{mntR44}::TT araC P_{araBAD} mntR | pG8R227 | Cm |
| ΔmntR28 | pYA3975 | Cm |

D. Mutations altering synthesis of LPS components

| | | |
|---|---|---|
| Δpmi-2426 | pYA3546 | Tet |
| ΔpagP8 | pYA4288 | Cm |
| ΔpagP81::Pₗₚₚ lpxE | pYA4295 | Cm |
| ΔpagL7 | pYA4284 | Cm |
| ΔlpxR9 | pYA4287 | Cm |
| ΔarnT6 | pYA4286 | Cm |
| ΔeptA4 | pYA4283 | Cm |
| ΔwaaC41 | pYA5473 | Cm |
| ΔwaaG42 | pYA4896 | Cm |
| ΔwaaL46 | pYA4900 | Cm |
| ΔwbaP45 | pYA4899 | Cm |
| ΔpagL64::TT araC P_{araBAD1} waaL | pYA5468 | Cm |
| ΔpagL64::TT rhaRS P_{rhaBAD1} waaL1 | pYA5377 | Cm |
| ΔpagL38::TT rhaRS P_{rhaBAD2} waaL2 | pG8R296 | Cm |

E. Mutations blocking catabolism of sugars

| | | |
|---|---|---|
| ΔaraBAD65::TT | pYA4811 | Cm |
| ΔrhaBADSR515 | pG8R272 | Cm |

F. Mutations altering synthesis of flagellar components

| | | |
|---|---|---|
| ΔfliC180 | pYA3729 | Cm |
| ΔfliC2426 | pYA3702 | Cm |
| ΔfljB217 | pYA3548 | Tet |
| Δ(hin-fljBA)-209 | pG8R306 | Cm |

G. Mutations altering synthesis of fimbrial components

| | | |
|---|---|---|
| Δ(agfG-agfC)-999 | pYA4941 | Cm |
| ΔP_{stc53}::P_{murA} stcA53 | pYA5053 | Cm |
| ΔstcABCD | pYA5007 | Tet |
| ΔP_{saf55}::P_{murA} safA55 | pYA5055 | Cm |
| ΔsafABCD | pYA4586 | Tet |

H. Mutations eliminating or altering outer membrane proteins

| | | |
|---|---|---|
| ΔompA11 | pYA4757 | Tet |

I. Mutations decreasing inflammation and enhancing mucosal immunity

| | | |
|---|---|---|
| ΔsopB1925 | pYA3733 | Cm |

J. Mutations eliminating or diminishing effective immunogenicity

| | | |
|---|---|---|
| ΔsifA26 | pYA3716 | Cm |

K. Mutations decreasing/delaying onset of pyroptosis

| | | |
|---|---|---|
| ΔsseL116 | pYA4621 | Cm |
| ΔtlpA181 | pYA4620 | Cm |

L. Mutations leading to degradation of DNA within Salmonella cells

| | | |
|---|---|---|
| ΔrecA62 | pYA4680 | Cm |
| ΔthyA | | |

^{a}Δ = deletion; TT = transcription terminator; P = promoter

TABLE 2
Mutations and associated phenotypes in S. Typhimurium adjuvant and vaccine vector strains^{a} It is noted
that the genes can be inactivated or deleted in multiple ways to confer the same phenotypic traits.
Also, though certain allele numbers are indicated elsewhere herein for certain mutations, reference
to a certain allele is not limiting and the mutations can be executed using other alleles.

| Genotype | Phenotype |
|---|---|
| ΔasdA | deletes gene for aspartate semialdehyde dehydrogenase essential for |
| | synthesis of diaminopimelic acid (DAP) necessary for peptidoglycan |
| | synthesis (58) |
| ΔP_{asdA}::TT araC P_{araBAD} asdA | makes synthesis of AsdA dependent on presence of arabinose |
| ΔP_{asdA77}::TT P_{rhaBAD1} asd | makes synthesis of AsdA dependent on presence of rhamnose |
| ΔP_{asdA88}::TT rhaRS P_{rhaBAD1} asd | makes synthesis of AsdA dependent on presence of rhamnose |
| ΔasdA::TT araC P_{araBAD} c2 | inactivates asdA and makes synthesis if C2 repressor |
| | dependent on arabinose (7, 59) |
| Δalr and ΔdadB | deletes the genes for two alanine racemases essential for synthesis |
| | of D-alanine necessary for peptidoglycan synthesis (60) |
| ΔP_{dadB}::TT araC P_{araBAD} dadB | makes synthesis of DadB dependent on presence of arabinose |
| ΔP_{dadB99}::TT P_{rhaBAD} dadB | makes synthesis of DadB dependent on presence of rhamnose |
| ΔP_{murA}::TT araC P_{araBAD} murA | makes synthesis of MurA, the first enzyme in the synthesis of |
| | muramic acid, dependent on arabinose in growth medium and ceases |
| | synthesis in vivo due to absence of arabinose (14, 18) |
| ΔP_{fur}::TT araC P_{araBAD} fur | makes synthesis of the Fur repressor protein dependent on |
| | arabinose in growth medium that ceases in vivo to result in high-level |
| | synthesis of all iron regulated proteins to result in attenuation (14, 15) |
| ΔP_{mntR}::TT araC P_{araBAD} mntR | makes synthesis of the MntR repressor protein dependent on |
| | arabinose in growth medium that ceases in vivo to result in high-level |
| | synthesis of all manganese regulated proteins |
| | to contribute to attenuation |
| ΔmntR28 | results in high-level synthesis of manganese-regulated proteins |
| | and some iron-regulated proteins |
| ΔaraBAD::TT | deletion of genes to eliminate arabinose catabolismwith TT inserted to |
| | prevent transcription of downstream genes (61-65) |
| ΔrhaBADSR | deletion of genes to eliminate rhamnose catabolism (66, 67) |
| ΔpagP::Pₗₚₚ lpxE mutation | causes regulated delayed in vivo synthesis of the codon-optimized |
| | lpxE gene from Francisella tularensis to cause synthesis of the non- |
| | toxic adjuvant form of LPS lipid A monophosphoryl lipid A (MPLA) |
| | (68) |
| ΔpagL and ΔlpxR | eliminates two means by which Salmonella alters LPS components in |
| | vivo to decrease recruitment of innate immunity by interaction with |
| | TLR4 (69) |
| ΔeptA | prevents addition of ethanolamine to lipid A (70, 71) |
| ΔarnT | prevents addition of 4-amino-4-deoxy-L-arabinose (L-Ara4N) groups to |
| | lipid A (72) |
| ΔfliC | deletes gene specifying synthesis of the phase I flagellin FliC (15, 73- |
| | 75) |
| ΔfljB | deletes gene specifying synthesis of the phase II flagellin FljB (15, 73- |
| | 75) |
| ΔfliC180 | specifies a truncated FliC protein containing TLR5 recognition domain |
| | and CD4 epitope (76); |
| Δ(hin-fljBA) | locks in expression of gene for phase I FliC flagellin and precludes |
| | synthesis of phase II FljB flagellin (77-83) |
| Δ(agfG-agfC) | deletes two operons specifying thin aggregative fimbriae (curli) and an |
| | activator for synthesis and export of cellulose and other |
| | exopolysaccharides (84) |
| ΔP_{saf5}::P_{murA} safA | causes constitutive synthesis of Saf fimbriae that facilitate spleen |
| | colonization (85) |
| ΔP_{stc}::P_{murA} stcA | causes constitutive synthesis of Stc fimbriae that facilitate spleen |
| | colonization (85) |
| ΔompA | specifies synthesis of a very prevalent outer membrane protein (86) |
| ΔsopB | a protein secreted by the Salmonella SPI-I that can cause intestinal |
| | inflammation (87-89) |
| Δpmi | eliminates phosphomannose isomerase that precludes synthesis of |
| | GDP-mannose that is needed for LPS O-antigen synthesis (15, 90) |
| ΔwaaC | encodes enzyme necessary for synthesis of the LPS inner core |
| | polysaccharide (84) |
| ΔwaaG | encodes enzyme necessary for synthesis of the LPS outer core |
| | polysaccharide (84) |
| ΔwbaP45 | encodes Und-P galactose phosphotransferase that produces the O- |
| | antigen ligase substrate to enable attachment of O-antigen to the LPS |
| | core by action of the O-antigen ligase encoded by waaL (84) |
| ΔwaaL & ΔpagL::TT araC P_{araBAD} waaL | make synthesis of the WaaL enzyme that couples O-antigen |
| (or ΔpagL::TT rhaRS P_{rhaBAD} waaL) | to the LPS core synthesis (91) dependent on presence of arabinose (or |
| | rhamnose) |
| Δ(wza-wcaM) | eliminates 20 genes encoding enzymes needed for synthesis of colonic |
| | acid, LPS capsular antigen and other polysaccharides to facilitate lysis, |
| | enhance immunogenicity and inhibit biofilm formation (92, 93) |
| ΔrelA | uncouples growth regulation from a dependence on protein synthesis |
| | (94, 95) |
| ΔrelA::araC P_{BAD} lacI TT and | makes synthesis of LacI that represses gene expression controlled |
| Δ(traM-traX)::araC P_{araBAD} lacI | by P_{trc} dependent on presence of |
| | arabinose (16, 17) with either inactivation of relA gene (75, 76) or |
| | deletion of genes encoding conjugational plasmid transfer in |
| | Salmonella virulence plasmid (96) |
| ΔspvRABCD | deletes Salmonella plasmid virulence genes encoding regulatory |
| | activator-repressor (R) and four genes conferring invasiveness and |
| | virulence; when the spvABCD genes are over expressed increase |
| | invasiveness and virulence |
| ΔcysG175::Pₛₚᵥ spvABCD | inserts spv operon without the R gene specifying the repressor- |
| | activator into a deletion of the cysG gene under control of a promoter |
| | not regulated by SpvR |
| ΔrecF | reduces inter- and intra-plasmidic recombination (60, 97-99) |
| ΔendA | deletes gene encoding endonuclease I to prevent degradation of |
| | released DNA vaccine (82) |
| ΔthyA | encodes thymidylate synthesis (100-104) |
| ΔsifA | enables Salmonella to escape from the SCV to enter the cytosol (105, |
| | 106) |
| ΔsseL | eliminates a gene that enables Salmonella to induce pyroptosis (82) |
| ΔtlpA | eliminates a gene that enables Salmonella to induce pyroptosis (82) |

^{a}Δ = deletion; TT = transcription terminator; P = promoter

### Example 3. Regulated Delayed Lysis Plasmid Vectors and Regulated Delayed Lysis DNA Vaccine Vectors for Use with PIESV Vector Strains

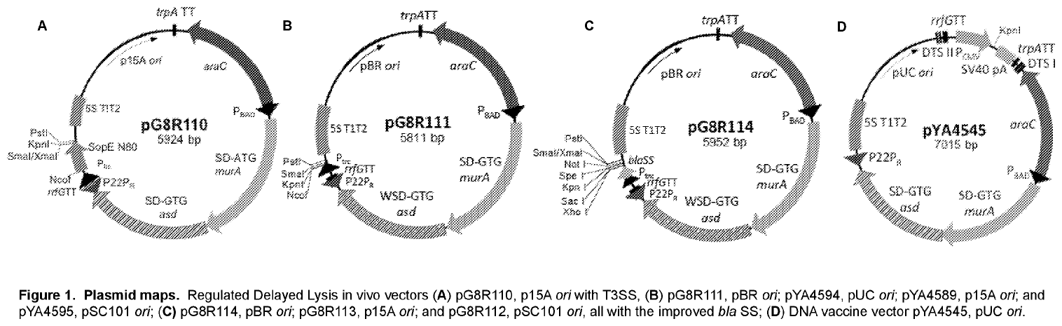

FIG. 1 provides diagrams of all the regulated lysis plasmid vectors (18-21, 107) with low pSC101 ori, moderate p15A ori, high pBR ori and very high pUC ori copy numbers. All plasmids to be used employ the balanced-lethal vector-host concept we developed (7) so that live PIESVs would be sensitive to all antibiotics and thus unable to disseminate antibiotic resistance when PIESV constructs are used in open environmental settings. The regulated lysis vectors (17,82) depicted in FIG. 1 have P_{trc} regulated gene expression to cause regulated delayed synthesis of protective antigens (due to gradual decrease in the quantity of LacI repressor specified by the chromosomal or virulence plasmid encoded arabinose-regulated araC P_{araBAD} lacI TT insertion mutations) for delivery by cell lysis and araC P_{araBAD}-regulated murA and asd genes with GTG start codons to decrease translation efficiency. The P22 P_{R} located with opposite orientation to the transcription of the araC P_{araBAD} GTG-murA GTG-asd genes is repressed by the C2 repressor made during growth of the strain with arabinose (due to the chromosomal ΔasdA27::TT araC P_{araBAD} c2 mutation). However, C2 concentration decreases due to cell division in vivo to cause P_{R}-directed synthesis of anti-sense mRNA to block translation of residual asdA and murA mRNA (18). As an alternate approach ensuring complete lysis, the ΔasdA27::TT araC P_{araBAD} c2 deletion-insertion mutation can be replaced by the ΔasdA33 mutation such that continued low-level transcription from the lysis plasmid P22 P_{R} (FIG. 1) is constant and reduces the amounts of the AsdA and MurA enzymes synthesized to result in an earlier cessation in peptidoglycan synthesis. Transcription terminators (TT) flank all plasmid domains for controlled lysis, replication and gene expression so that expression in one domain does not affect expression in another domain. A multiple insertion site is placed after the P_{trc} sequence with or without T2 or T3 secretion sequences for fusion to protein antigens encoded by inserted DNA sequences. The regulated delayed lysis DNA vaccine vector pYA4545 (82) (FIG. 1) possesses some of the same attributes as the plasmid vectors for regulated lysis. In addition, sequences subject to attack and digestion by host nucleases have been eliminated while multiple sequences facilitating nuclear targeting in host cells (an attribute totally lacking in bacterial plasmids) have been added. The CMV promoter and SV40 polyA termination sequences for eukaryotic transcription initiation and termination have also been improved (107). As with the regulated lysis plasmid vectors, transcription terminators (TT) flank all domains for regulatory functions (18).

### Example 4. Design and Construction of the Parent of the Family 1 S. typhimurium UK-1 PIESV Vector Strain

The PIESV vector strain χ12495 was selected for further modification. This strain when used with any of the regulated delayed lysis plasmid vectors depicted in FIG. 1 will display the regulated delayed lysis in vivo phenotype due to the absence of arabinose needed for synthesis of aspartate semialdehyde dehydrogenase encoded by the asdA gene and UDP-N-acetylglucosamine enolpyruvyl transferase encoded by the murA gene necessary for the synthesis of diaminopimelic acid (DAP) and muramic acid, respectively, two unique essential components of the peptidoglycan rigid layer of the bacterial cell wall. In the absence of arabinose, these strains ultimately undergo lysis as the asdA- and murA-encoded enzymes are diluted as a consequence of PIESV cell division in vivo. Although Salmonella has a generation time of some 40 to 50 min when growing in the lab in LB broth, cell division in vivo takes much longer varying from 10 to 20 h depending on the specific in vivo intra or extracellular habitat. In addition, we have investigated many means to modulate the rate of in vivo cell division of Salmonella vaccine vector strains and discovered genotypes to enable sufficiently prolonged persistence with protective antigen synthesis and delivery to maximize induction of mucosal, systemic and cellular protective immune responses but at the same time to ensure expression of biological containment with no long-term persistence of viable PIESV cells in vivo and no survival if shed into the environment. These collective attributes are unique and important, since persistence of viable Salmonella vaccine cells for two to three weeks in vivo is critical for the induction of cellular immune responses of long duration due to induction of immune memory responses (108). On the other hand, demise of viable vaccine cells within five to six weeks is very important when PIESV constructs are used to vaccinate broiler chickens that go to market at about six weeks of age since it is undesirable to have viable vaccine cells contaminate carcasses destined for human consumption. Thus, many of these newly discovered improvements have been added in the χ12615 genome in its derivation from the starting strain χ12495 as well as in the further improvements of χ12615 to generate χ12688 and χ12702. Although many of the mutations originally in χ12495 have been eliminated or replaced with substitutions, certain beneficial mutations present in χ12495 have been retained. These include Δ(wza-wcaM)-8 that eliminates some 20 genes that encode enzymes that Salmonella can use to synthesize extracellular polysaccharides such as colanic acid (25) that is made under stress and reduces completeness of regulated cell lysis (109-113) and LPS O-antigen capsule (114-116) that both act to suppress induction of immune responses. In addition, the Δ(wza-wcaM)-8 mutation enhances early onset induced antibody responses (25). The ΔrecF126 mutation reduces inter- and intra-plasmidic recombination and thus stabilizes maintenance of the genetic integrity of regulated lysis plasmid vectors (99). The ΔsifA26 mutation enables PIESV constructs to escape from the Salmonella Containing Vacuole (SCV, an endosome compartment) so that lysis of some vaccine cells occurs in the cytosol so that protective antigens released during lysis can be presented to the proteasome for Class I presentation to facilitate induction of CD8, CD17 and NKT cellular immune responses (20-23). Other mutations changed or added during the construction of χ12615, χ12688 and χ12702 are described below but detailed studies discovering newly developed strategies are described and justified more fully in succeeding Examples. Table 3 lists the steps in constructing the Parental Family 1 strains χ12615, χ12688 and χ12702 using the suicide vectors listed in Table 1 for introduction of mutations listed and described in Table 2.

TABLE 3
Derivation of the parent of the Family 1 S. Typhimurium UK-1 PIESV
vector strains χ12615, χ12688 and χ12702^{a}.

χ12495 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
χ12509 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
ΔaraBAD65::TT (from χ12495 by using suicide vector pYA4811 to introduce the
ΔaraBAD65::TT deletion mutation to prevent catabolism of arabinose)
χ12527 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
ΔaraBAD65::TT ΔrhaBADSR515 (from χ12509 by using suicide vector pG8R272 to introduce
the ΔrhaBADSR515 deletion mutation to prevent catabolism of rhamnose)
χ12531 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA197::araC P_{araBAD} lacI TT ΔrecF126 ΔsifA26
ΔaraBAD65::TT ΔrhaBADSR515 (from χ12527 by using suicide vector pG8R296 to substitute
ΔpagL38::TT rhaRS P_{rhaBAD2} waaL2 deletion-insertion mutation to replace ΔpagL64::TT rhaRS
P_{rhaBAD1} waaL1 deletion-insertion mutation)
χ12561 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA197::araC P_{araBAD} lacI TT ΔrecF126 ΔsifA26
ΔaraBAD65::TT ΔrhaBADSR515 ΔlpxR9 (from χ12531 by using suicide vector pYA4287 to
introduce ΔlpxR9 deletion mutation)
χ12562 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA197::araC P_{araBAD} lacI TT ΔrecF126 ΔsifA26
ΔaraBAD65::TT ΔrhaBADSR515 ΔpagP8 (from χ12531 by using suicide vector pYA4288 to
introduce ΔpagP8 deletion mutation)
χ12569 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA197::araC P_{araBAD} lacI TT ArecF126 ΔsifA26
ΔaraBAD65::TT ΔrhaBADSR515 ΔpagP8 ΔlpxR9 (from χ12562 by using suicide vector
pYA4287 to introduce ΔlpxR9 deletion mutation)
12602 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA1123 ΔrecF126 ΔsifA26 ΔaraBAD65::TT
ΔrhaBADSR515 ΔpagP8 ΔlpxR9 (from χ12569 by using suicide vector pYA3679 to substitute
ΔrelA1123 deletion mutation for ΔrelA197::araC P_{araBAD} lacI TT deletion-insertion mutation)
χ12615 ΔP_{murA25}::TT araC P_{araBAD} murA ΔasdA27::TT araC P_{araBAD} c2 ΔwaaL46 ΔpagL38:TT
rhaRS P_{rhaBAD2} waaL2 Δ(wza-wcaM)-8 ΔrelA1123 ΔrecF126 ΔsifA26 ΔaraBAD65::TT
ΔrhaBADSR515 ΔpagP8 ΔlpxR9 (pSTUK201 Δ(traM-traX)-36::araC P_{araBAD} lacI TT) (from
χ12602 by replacing the pSTUK100 conjugative virulence plasmid with the non-conjugative
pSTUK201 derivative with the araC P_{araBAD} lacI TT insertion modification)
χ12663 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA1123 ΔrecF126 ΔsifA26 ΔaraBAD65::TT
ΔrhaBADSR515 ΔpagP8 ΔlpxR9 (pSTUK206 Δ(traM-traX)-41::araC P_{araBAD} lacI TT) (from
χ12615 by replacement of the sequence in the regulated lacI constructions using suicide vector
pG8R381 to decrease the amount of LacI synthesized induced by arabinose)
χ12667 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA1123 ΔrecF126 ΔsifA26 ΔaraBAD65::TT
ΔrhaBADSR515 ΔpagP8 ΔlpxR9 ΔompA11 (pSTUK206 Δ(traM-traX)-41::araC P_{araBAD} lacI TT
(from χ12663 to delete the ompA gene using suicide vector pYA4757 to enhance immunity to
other important outer membrane protein antigens)
χ12688 ΔP_{murA25}::TT araC P_{araBAD} murA ΔasdA33 ΔwaaL46 ΔpagL38:TT rhaRS P_{rhaBAD2}
waaL2 Δ(wza-wcaM)-8 ΔrelA1123 ΔrecF126 ΔsifA26 ΔaraBAD65::TT ΔrhaBADSR515 ΔpagP8
ΔlpxR9 (pSTUK206 Δ(traM-traX)-41::araC P_{araBAD} lacI TT) (from χ12663 to replace the
ΔasdA27::TT araC P_{araBAD} c2 deletion-insertion mutation with the ΔasdA33 mutation using
suicide vector pYA3736 to enhance complete lysis of vaccine cells)
χ12698 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA1123 ΔrecF12 ΔsifA26 ΔaraBAD65::TT
ΔrhaBADSR515 ΔpagP81::Pₗₚₚ lpxE ΔlpxR9 (pSTUK206 Δ(traM-traX)-41::araC P_{araBAD} lacI
TT) (from χ12663 by replacing the ΔpagP8 mutation with the ΔpagP81::Pₗₚₚ lpxE deletion-
insertion mutation using the suicide vector pYA4295 to replace the toxic lipid A with the
adjuvant non-toxic MPLA))
χ12702 ΔP_{murA25}::TT araC P_{araBAD} murA ΔasdA33 ΔwaaL46 ΔpagL38::TT rhaRS P_{rhaBAD2}
waaL2 Δ(wza-wcaM)-8 ΔrelA1123 ΔrecF12 ΔsifA26 ΔaraBAD65::TT ΔrhaBADSR515
ΔpagP81::Pₗₚₚ lpxE ΔlpxR9 (pSTUK206 Δ(traM-traX)-41::araC P_{araBAD} lacI TT) (from χ12698
to replace the ΔasdA27::TT araC P_{araBAD} c2 deletion-insertion mutation with the ΔasdA33
mutation using suicide vector pYA3736 to enhance complete lysis of vaccine cells by
eliminating the synthesis of the C2 repressor that can repress synthesis of anti-sense mRNA
directed by the P22 P_{R} on the regulated delayed lysis plasmid vectors)
^{a}Phenotypes associated with mutations are defined in Table 1 and suicide vectors used in constructions are listed in Table 2.

### Example 5. Design and Construction of the Parent of the Family 2 S. typhimurium UK-1 PIESV Vector Strains χ12616 and χ12704

The construction of strains listed in Table 4 was designed to yield χ12616/χ12704 with capabilities to induce cross-protective immunity to enteric bacterial species by inducing immune responses to iron- and manganese-regulated proteins and outer membrane proteins, many of which share common structural and immunological attributes and the LPS core polysaccharide that is identical in essentially all S. enterica serotypes. Exposure of these cell surface macromolecules was achieved by regulated delayed loss of the surface-covering LPS O-antigen. These strains were constructed using the suicide vectors listed in Table 1 for introduction of mutations listed and described in Table 2. These strains can be used with any of the regulated delayed lysis plasmid vectors depicted in FIG. 1 to encode synthesis and delivery of additional protective antigens. These strains will display the regulating delayed lysis in vivo phenotype.

TABLE 4
Derivation of the parent of the Family 2 S.
Typhimurium UK-1 PIESV vector strain χ12616^{a}.

χ12495 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
χ12510 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
P_{mntR44}::TT araC P_{araBAD} mntR (from χ12495 by using suicide vector pG8R227 to introduce
P_{mntR44}::TT araC P_{araBAD} mntR deletion-insertion mutation)
χ12533 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
P_{mntR44}::TT araC P_{araBAD} mntR ΔP_{fur33}::TT araC P_{araBAD} fur (from χ12510 by using suicide vector
pYA3722 to introduce ΔP_{fur33}::TT araC P_{araBAD} fur deletion-insertion mutation)
χ12538 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
P_{mntR44}::TT araC P_{araBAD} mntR ΔP_{fur33}::TT araC P_{araBAD} fur ΔaraBAD65::TT (from χ12533 by
using suicide vector pYA4811 to introduce the ΔaraBAD65::TT deletion mutation to prevent
catabolism of arabinose)
χ12540 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA197::araC P_{araBAD} lacI TT ΔrecF126 ΔsifA26
P_{mntR44}::TT araC P_{araBAD} mntR ΔP_{fur33}::TT araC P_{araBAD} fur ΔaraBAD65::TT ΔrhaBADSR515
(from χ12538 by using suicide vector pG8R272 to introduce the ΔrhaBADSR515 deletion
mutation to prevent catabolism of rhamnose)
χ12541 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA197::araC P_{araBAD} lacI TT ΔrecF126 ΔsifA26
P_{mntR44}::TT araC P_{araBAD} mntR ΔP_{fur33}::TT araC P_{araBAD} fur ΔaraBAD65::TT ΔrhaBADSR515
(from χ12540 by using suicide vector pG8R296 to replace ΔpagL64::TT rhaRS P_{rhaBAD1} waaL1
with the ΔpagL38::TT rhaRS P_{rhaBAD2} waaL2 deletion-insertion mutation)
χ12607 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA1123 ΔrecF126 ΔsifA26 P_{mntR44}::TT araC P_{araBAD}
mntR ΔP_{fur33}::TT araC P_{araBAD} fur ΔaraBAD65::TT ΔrhaBADSR515 (from χ12541 by using
suicide vector pYA3679 to substitute ΔrelA1123 deletion mutation for
ΔrelA197::araC P_{araBAD} lacI TT deletion-insertion mutation)
χ12616 ΔP_{murA25}::TT araC P_{araBAD} murA ΔasdA27::TT araC P_{araBAD} c2 ΔwaaL46 ΔpagL38::TT
rhaRS P_{rhaBAD2} waaL2 Δ(wza-wcaM)-8 ΔrelA1123 ΔrecF126 ΔsifA26 P_{mntR44}::TT araC P_{araBAD}
mntR ΔP_{fur33}::TT araC P_{araBAD} fur ΔaraBAD65::TT ΔrhaBADSR515 (pSTUK201 Δ(traM-traX)-
36:: araC P_{araBAD} lacI TT) from χ12607 by replacing the pSTUK100 conjugative virulence
plasmid with the non-conjugative pSTUK201 derivative with the araC P_{araBAD} lacI TT insertion
modification)
χ12664 ΔP_{murA25}::TT araC P_{araBAD} murA ΔasdA27::TT araC P_{araBAD} c2 ΔwaaL46 Δ(wza-wcaM)-
8 ΔrelA1123 ΔrecF126 ΔsifA26 P_{mntR44}::TT araC P_{araBAD} mntR ΔP_{fur33}::TT araC P_{araBAD} fur
ΔaraBAD65:TT ΔrhaBADSR515 ΔpagL38::TT rhaRS P_{rhaBAD2} waaL2 (pSTUK206 Δ(traM-
traX)-41::araC P_{araBAD} lacI TT) (from χ12616 by replacement of the sequence in the regulated
lacI construction using suicide vector pG8R381 to decrease the amount of LacI synthesized)
χ12689 ΔP_{murA25}::TT araC P_{araBAD} murA ΔasdA33 ΔwaaL46 Δ(wza-wcaM)-8 ΔrelA1123
ΔrecF126 ΔsifA26 P_{mntR44}::TT araC P_{araBAD} mntR ΔP_{fur33}::TT araC P_{araBAD} fur ΔaraBAD65::TT
ΔrhaBADSR515 ΔpagL38::TT rhaRS P_{rhaBAD2} waaL2 (pSTUK206 Δ(traM-traX)-41::araC
P_{araBAD} lacI TT) (from χ12664 to replace the ΔasdA27::TT araC P_{araBAD} c2 deletion-insertion
mutation with the ΔasdA33 mutation by using the suicide vector pYA3736 to enhance complete
lysis of vaccine cells by eliminating the synthesis of the C2 repressor that can repress synthesis
of anti-sense mRNA directed by the P22 P_{R} on the regulated delayed lysis plasmid vectors)
χ12704 ΔP_{murA25}::TT araC P_{araBAD} murA ΔasdA33 ΔwaaL46 ΔpagL38::TT rhaRS P_{rhaBAD2}
waaL2 Δ(wza-wcaM)-8 ΔrelA1123 ΔrecF126 ΔsifA26 ΔmntR28 ΔP_{fur33}::TT araC P_{araBAD} fur
ΔaraBAD65::TT ΔrhaBADSR515 (pSTUK206 Δ(traM-traX)-41::araC P_{araBAD} lacI TT)
(from χ12689 to replace using the suicide vector pYA3975 the P_{mntR44}::TT araC P_{araBAD} mntR
deletion-insertion mutation with the ΔmntR28 mutation, which does not attenuate but ensures
derepression of Mn-regulated genes and some co-regulated by Fur to further enhance display of
IROMPs)
^{a}Phenotypes associated with mutations are defined in Table 1 and suicide vectors used in constructions are listed in Table 2.

### Example 6. Design and Construction of the Parent of the Family 3 S. typhimurium UK-1 PIESV DNA Vaccine Vector Strains χ12601, χ12705 and χ12706

The improved strain χ12601 whose derivation is listed in Table 5 was derived from χ12378. This strain was designed to optimally transfer DNA vaccine vectors encoding protective antigens to be synthesized in the vaccinated animal or human host. χ12601 is best suited for combination with the regulated delayed lysis DNA vaccine vector pYA4545 (FIG. 1) to encode sequences for synthesis of protective antigens. The DNA vaccine vector strain χ12601 has desirable properties also displayed by the Family 1 and Family 2 strains but in addition possesses the ΔendA2113 mutation to preclude cleavage of the DNA vaccine vector by the bacterial periplasmic endonuclease during its release from the PIESV DNA vaccine donor strain displaying regulated in vivo lysis. In addition, strain χ12601 possesses the ΔsseL116 and ΔtlpA181 deletion mutations that eliminate two gene functions responsible for Salmonella-induced pyroptosis that leads to the destruction of the host cell nuclear apparatus to very much reduce expression of the DNA vaccine vector-encoded protective antigens due to impairment in transcription and subsequent translation. The suicide vectors listed in Table 1 were used for the introduction of the mutations listed and described in Table 2. During the construction of these strains, we determined that the timing of lysis was influenced depending on which means of cessation in waaL expression was used. We therefore decided to co-develop χ12563 (with ΔwaaL46 ΔpagL64::TT rhaRS P_{rhaBAD1} waaL1) and χ12601 (with ΔwaaL46 ΔpagL38::TT rhaRS P_{rhaBAD1} waaL2) We thus made derivatives of both χ12563 and χ12601 with the ΔaraBAD65::TT and ΔrhaBADSR515 mutations that delay onset of in vivo lysis to determine which timing is best for delivery of DNA vaccine vectors to maximize induction of protective immunity. The last step was to replace the ΔasdA27::TT araC P_{araBAD} c2 mutation with the ΔasdA33 mutation yielding the strains χ12705 and χ12706.

TABLE 5
Derivation of the parents of the Family 3 S. Typhimurium UK-1 PIESV DNA vaccine
vector strains χ12563, χ12601, χ12705 and χ12706^{a}.

χ12378 ΔP_{murA25}::TT araC P_{araBAD} murA ΔasdA27::TT araC P_{araBAD} c2 Δpmi-2426 ΔwaaL46
ΔpagL64::TT rhaRS P_{rhaBAD1} waaL1 Δ(wza-wcaM)-8 ΔrelA197::araC P_{araBAD} lacI TT ΔrecF126
ΔsifA26 ΔendA2113
χ12380 ΔP_{murA25}::TT araC P_{araBAD} murA ΔasdA27::TT araC P_{araBAD} c2 Δpmi-2426 ΔwaaL46
ΔpagL64::TT rhaRS P_{rhaBAD1} waaL1 Δ(wza-wcaM)-8 ΔrelA1123 ΔrecF126 ΔsifA26 ΔendA2113
(from χ12378 by using suicide vector pYA3679 to introduce ΔrelA1123 deletion mutation)
χ12384 ΔP_{murA25}::TT araC P_{araBAD} murA ΔasdA27::TT araC P_{araBAD} c2 Δpmi-2426 ΔwaaL46
ΔpagL64::TT rhaRS P_{rhaBAD1} waaL1 Δ(wza-wcaM)-8 ΔrelA1123 ΔrecF126 ΔsifA26 ΔendA2113
ΔsseL116 (from χ12380 by using suicide vector pYA4621 to introduce ΔsseL116 deletion
mutation)
χ12388 ΔP_{murA25}::TT araC P_{araBAD} murA ΔasdA27::TT araC P_{araBAD} c2 Δpmi-2426 ΔwaaL46
ΔpagL64::TT rhaRS P_{rhaBAD1} waaL1 Δ(wza-wcaM)-8 ΔrelA1123 ΔrecF126 ΔsifA26 ΔendA2113
ΔsseL116 ΔtlpA181 (from χ12384 by using suicide vector pYA4620 to introduce ΔtlpA181
deletion mutation)
χ12563 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA1123 ΔrecF126 ΔsifA26 ΔendA2113 ΔsseL116
ΔtlpA181 (from χ12388 by replacement of the Δpmi-2426 deletion mutation with its wild-type
allele using P22 transduction propagated on χ3761)
χ12601 ΔP_{murA25}::TT araC P_{araBAD} murA ΔasdA27::TT araC P_{araBAD} c2 ΔwaaL46 ΔpagL38::TT
rhaRS P_{rhaBAD1} waaL2 Δ(wza-wcaM)-8 ΔrelA1123 ΔrecF126 ΔsifA26 ΔendA2113 ΔsseL116
ΔtlpA181 (from χ12563 by using suicide vector pG8R296 to replace ΔpagL64::TT rhaRS
P_{rhaBAD1} waaL1 with the ΔpagL38::TT rhaRS P_{rhaBAD2} waaL2 deletion-insertion mutation)
χ12642 ΔP_{murA25}::TT araC P_{araBAD} murA ΔasdA27::TT araC P_{araBAD} c2 ΔwaaL46 ΔpagL38::TT
rhaRS P_{rhaBAD1} waaL2 Δ(wza-wcaM)-8 ΔrelA1123 ΔrecF126 ΔsifA26 ΔendA2113 ΔsseL116
ΔtlpA181 ΔrhaBADSR515 (from χ12601 by using suicide vector pG8R272 to introduce the
ΔrhaBADSR515 deletion mutation to prevent catabolism of rhamnose)
χ12643 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA1123 ΔrecF126 ΔsifA26 ΔendA2113 ΔsseL116
ΔtlpA181 ΔrhaBADSR515 (from χ12563 by using suicide vector pG8R272 to introduce the
ΔrhaBADSR515 deletion mutation to prevent catabolism of rhamnose)
χ12644 ΔP_{murA25}::TT araC P_{araBAD} murA ΔasdA27::TT araC P_{araBAD} c2 ΔwaaL46 ΔpagL38::TT
rhaRS P_{rhaBAD1} waaL2 Δ(wza-wcaM)-8 ΔrelA1123 ΔrecF126 ΔsifA26 ΔendA2113 ΔsseL116
ΔtlpA181 ΔrhaBADSR515 ΔaraBAD65::TT (from χ12642 by using suicide vector pYA4811 to
introduce the ΔaraBAD65::TT deletion mutation to prevent catabolism of arabinose)
χ12645 ΔP_{murA25}::TT araC P_{araBAD} murA ΔasdA27::TT araC P_{araBAD} c2 ΔwaaL46 ΔpagL64::TT
rhaRS P_{rhaBAD1} waaL1 Δ(wza-wcaM)-8 ΔrelA1123 ΔrecF126 ΔsifA26 ΔendA2113 ΔsseL116
ΔtlpA181 ΔrhaBADSR515 ΔaraBAD65::TT (from χ12643 by using suicide vector pYA4811 to
introduce the ΔaraBAD65::TT deletion mutation to prevent catabolism of arabinose)
χ12705 ΔP_{murA25}::TT araC P_{araBAD} murA ΔasdA33 ΔwaaL46 ΔpagL38::TT rhaRS P_{rhaBAD2}
waaL2 Δ(wza-wcaM)-8 ΔrelA1123 ΔrecF126 ΔsifA26 ΔendA2113 ΔsseL116 ΔtlpA181
ΔrhaBADSR515 ΔaraBAD65::TT (from χ12644 to replace the ΔasdA27::TT araC P_{araBAD} c2
deletion-insertion mutation with the ΔasdA33 mutation using the suicide vector pYA3736 to
enhance complete lysis of vaccine cells by eliminating the synthesis of the C2 repressor that can
repress synthesis of anti-sense mRNA directed by the P22 P_{R} on the regulated delayed lysis
plasmid vectors)
χ12706 ΔP_{murA25}::TT araC P_{araBAD} murA ΔasdA33 ΔwaaL46 ΔpagL64::TT rhaRS P_{rhaBAD1}
waaL1 Δ(wza-wcaM)-8 ΔrelA1123 ΔrecF126 ΔsifA26 ΔendA2113 ΔsseL116 ΔtlpA181
ΔrhaBADSR515 ΔaraBAD65::TT (from χ12645 to replace the ΔasdA27::TT araC P_{araBAD} c2
deletion-insertion mutation with the ΔasdA33 mutation using the suicide vector pYA3736 to
enhance complete lysis of vaccine cells by eliminating the synthesis of the C2 repressor that can
repress synthesis of anti-sense mRNA directed by the P22 P_{R} on the regulated delayed lysis
plasmid vectors)

### Example 7. Further Potential Modifications of the PIESV Strains χ12615, χ12688 and χ12702 (Family 1), χ12616 and χ12704(Family 2) and/or χ12563, χ12601, χ12705 and χ12706 (Family 3) to Impart Other Beneficial Attributes to Enhance Effectiveness as PIESV Antigen (Families 1 and 2) or DNA Vaccine (Family 3) Delivery Vectors

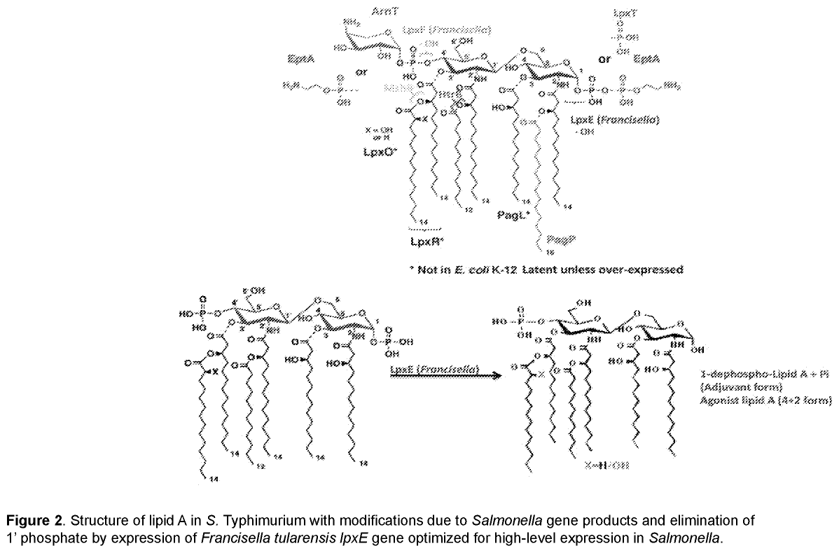

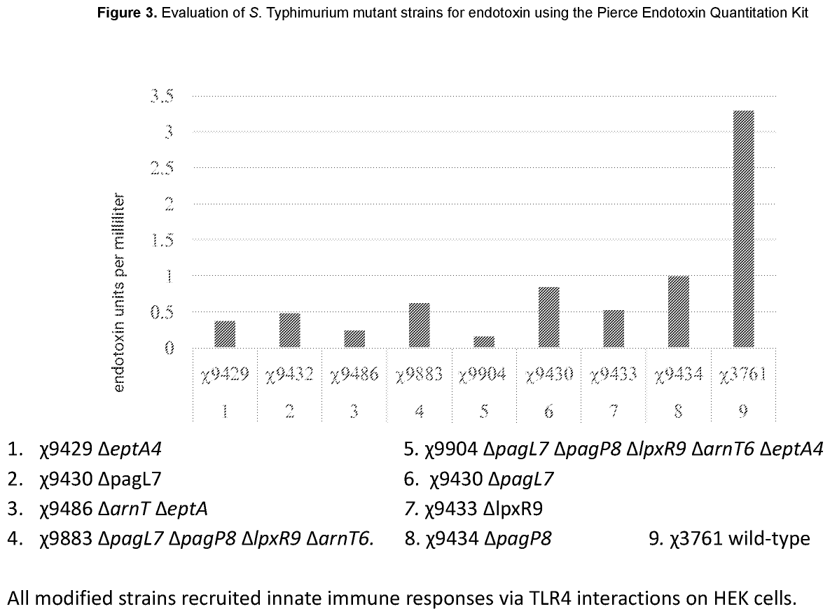

Based on prior studies and discoveries plus well-established facts it will be appropriate to further modify vaccine vector strains to tailor them for specific applications or for specific host species. It is well recognized that animal sensitivity to the endotoxin of gram-negative bacteria composed of the LPS lipid A moiety varies over a 1000-fold range with birds such as chickens being very tolerant/resistant while humans and especially horses are extremely sensitive (117-122). Since deletion of genes for synthesis of lipid A is lethal due to the importance of lipid A in constituting the lipid in the outer layer of the outer membrane in gram negative bacteria, it is critical to determine the components of lipid A leading to toxicity to determine how they might be modified or eliminated to reduce toxicity. However, lipid A constitutes a Pathogen-Associated Molecular Pattern (PAMP) now generally referred to as a Microbe-Associated Molecular Pattern (MAMP) since they are present on all gram-negative bacteria) that interacts with TLR4 to recruit innate immunity. This interaction is therefore of critical importance in enhancing the immunogenicity of bacterial vectored vaccines. Since the 1′ and 4′ phosphates attached to the carbohydrate backbone of lipid A are essential for toxicity and virulence (69), we chose to delete one of these phosphates by over expression in S. typhimurium of a codon-optimized lpxE gene of Francisella tularensis to generate a strain that made a non-toxic mono-phosphoryl lipid A that serves as a potent adjuvant to enhance immunogenicity by activating TLR4 via a non-inflammatory pathway (68). We can thus add the ΔpagP81::Pₗₚₚ lpxE deletion-insertion mutation into PIESV vector strains so that lysis liberates a non-inflammatory, non-toxic adjuvant form of lipid A to enhance immunogenicity, especially when used to vaccinate animal or human hosts sensitive to the wild-type lipid A. The structure of lipid A due to this modification is diagramed in the bottom half of FIG. 2. Salmonella, like most pathogens has evolved means to suppress or modulate the infected host's ability to respond with a protective immune response. Many of these evasive immunosuppressive mechanisms have been by modification of lipid A to decrease its ability to interact with TLR4 to stimulate an innate immunity to enhance induction of protective immunity to clear the infection. These modifications and the enzymes responsible for each modification are diagrammed in the top of FIG. 2. These ΔlpxR9, ΔpagL7, ΔpagP8, ΔeptA4 and ΔarnT6 deletion mutations listed in Table 2, individually and in combination decrease or eliminate lipid A toxicity (FIG. 3) to enhance a productive interaction of the modified lipid A to interact with TLR4 on the surface of host cells. Since χ9904 with the ΔlpxR9, ΔpagL7, ΔpagP8, ΔeptA4 and ΔarnT6 deletion mutations displays the least toxicity in the Pierce Test (FIG. 3), the ΔeptA4 and ΔarnT6 mutations could be added to the improved Family 1 PIESV strains χ12688 and χ12702 as well as all five mutations to Family 2 strains. However, this was not deemed necessary since the Family B strains have most useful applications in the poultry industry wherein the bird have a very high tolerance to LPS lipid A toxicity.

Since some hosts to be orally vaccinated with PIESV constructs might have increased susceptibility to intestinal inflammation, a likelihood if orally vaccinating newborns and infants, the PIESV vector strains can be further modified by introduction of the ΔsopB1925 mutation that significantly reduces inflammation in the small intestine (123). This deletion has the additional benefit since the SopB protein is immunosuppressive (124). Importantly, mucosal vaccination with attenuated Salmonella vaccine strains that possess the ΔsopB1925 mutation are superior in inducing elevated mucosal immune responses (24). In this regard, several derivatives of strains listed in Tables 3 and 5 have been constructed that have both the ΔsopB1925 and ΔompA11 (see below) mutations.

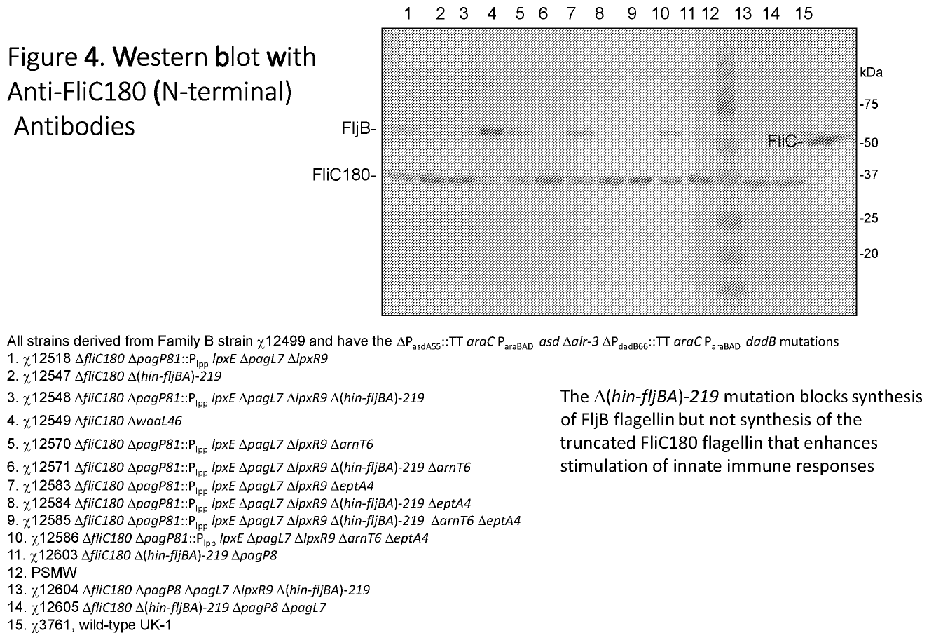

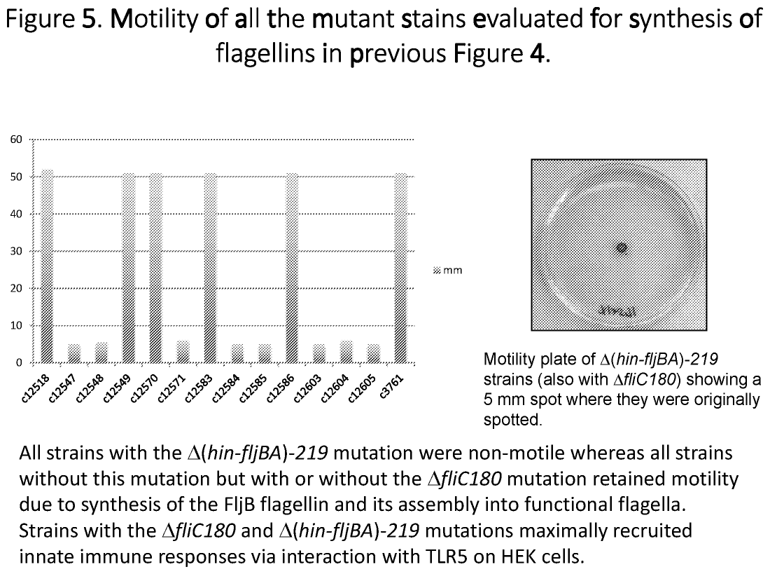

Salmonella is motile due to flagella made by synthesis and assembly of two types of antigenically distinct flagellins that can be alternately synthesized by an invertible switch hin that enables synthesis of the Phase I FliC flagellin or the Phase II FljB flagellin. This phase variation occurs spontaneously at a rate of about 10⁻⁴ per cell division (59,62). Unassembled FliC flagellin secreted by Salmonella interacts with the cell surface associated TLR5 on cells in a vaccinated or infected animal or human host to activate expression of innate immunity. Salmonella can reduce this stimulation of innate immunity by switching to synthesis of the Phase II FljB flagellin. Of importance in modifying PIESV strains to enhance recruitment of innate immunity is the fact that neither flagella nor motility due to the presence of flagella is necessary for Salmonella to infect animal hosts when administered by a mucosal route such as orally (125). This is of critical importance since it is the unassembled flagellin, and not flagella or partially aggregated flagellin subunits, that interact with TLR5 (126). Thus, PIESV strains can be modified to synthesize and uniformly secrete a FliC subunit that possesses the TLR5 binding domain in addition to a CD4 T-cell epitope by including the ΔfliC180 and Δ(hin-fljBA)-219 deletion mutations. The ΔfliC180 mutation encodes such a flagellin that is unable to aggregate and thus all molecules synthesized and secreted are available to activate innate immune responses via interaction with TLR5 on the surface of cells in the vaccinated animal host. FIG. 4 presents data from western blot analyses of S. typhimurium mutant strains with combinations of mutations governing synthesis of the FliC180 truncated protein with mutations blocking phase variation and synthesis of the FljB flagellin and with various modification of lipid A. FIG. 5 analyzes the same strains as used for the FIG. 4 data to show motility or non-motility dependent on the genotype with only strains able to synthesize the FljB flagellin being able to display motility.

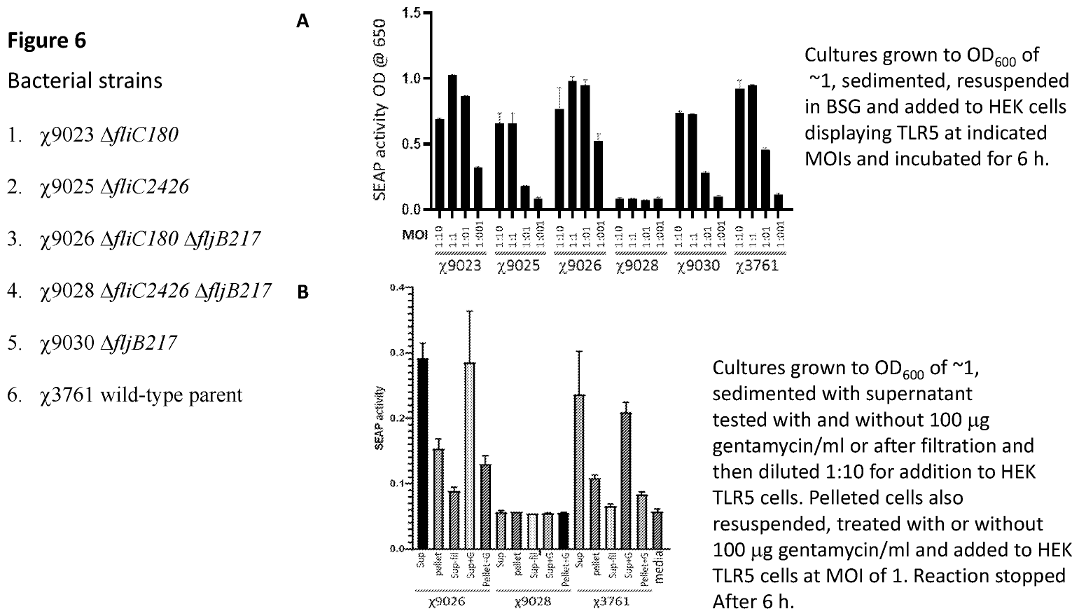

In analyzing strains for interaction with TLR5 on HEK cells, strains that only synthesize FljB (χ9025) or FliC (χ9030) are able to recruit innate immune responses, but to a lesser extent than the wild-type strain χ3761 able to synthesize either phase I or II flagellin (FIG. 6A). A strain with deletion of both the fliC and fljB genes (χ9028) is, of course, inactive in interacting with TLR5 on HEK cells. Strains with the ΔfliC180 mutation (χ9023 and χ9026) synthesize a truncated FliC protein unable to form flagella or exhibit motility (FIG. 5) but are very efficient in interacting with TLR5 on HEK cells to activate innate immunity (FIG. 6A). The synthesis of flagella in Salmonella is dependent, in part, on the synthesis and assembly of a type 3 secretion system that secretes flagellin monomers to be assembled into flagella on the exterior cell surface. To verify that strains with the ΔfliC180 mutation secreted the flagellin monomer, we conducted a cell fractionation experiment comparing cell culture supernatant fractions after sedimentation of cells. We thus compared χ9026 (producing the FliC180 truncated flagellin), χ9028 (no flagellin production) and the wild-type parent χ3761. Since sedimentation of bacterial cells by centrifugation only reduces cells remaining in the supernatant about 1000-fold, we compared filtration of supernatants versus killing residual cells in the supernatant with gentamycin. As shown by the data in FIG. 6B, the supernatant fraction treated with gentamycin revealed that most activity of the χ9026 culture resided in the supernatant. This conclusion is augmented by the finding that resuspended bacteria after their pelleting by centrifugation had a very reduced ability to interact with TLR5 on HEK cells, with or without treatment with gentamycin (FIG. 6B).

Based on the results of these studies, it will be best to combine use of the ΔfliC180 mutation with the Δ(hin-fljBA)-219 mutation so that the fliC gene is expressed by 100 percent of cells in the population so as to maximize recruitment of TLR5 to activate induction of innate immunity. This strategy has been used in the design and construction of the Family A and B SDAAS strains used as live adjuvants described in Example 20. The inclusion of these two mutations in any of the improved Family 1, 2 and 3 strains can readily be accomplished to enhance recruitment of innate immunity by activation via TLR5 by using the suicide vectors listed in Table 1.

S. typhimurium has some 12 operons encoding fimbrial appendages. Some of these fimbriae contribute to intestinal colonization because of adherent components on the fimbriae. However, some of these fimbriae fail to be synthesized under any in vitro condition and are not synthesized in the GI tract either. However, the Sta and Saf fimbriae that are not synthesized under any laboratory experimental condition are synthesized and assembled in vivo in spleens (85). This in vivo up-regulation in synthesis and assembly was an important observation since the spleen is possibly the most important internal effector lymphoid tissue and is responsible for generating long-lasting protective immunity. Constitutive in vivo synthesis of either the Saf fimbriae or the Stc fimbriae in PIESV strains can be achieved by the addition of either the ΔP_{saf5}::P_{murA} safA or ΔP_{stc}::P_{murA} stcA deletion-insertion mutation, in which synthesis and assembly is specified by the constitutive promoter of the mur operon, essential for synthesis of the rigid peptidoglycan layer of the cell wall. Such PIESV vaccine vector strains delivering the protective Streptococcus pneumoniae PspA protective antigen enhanced both the anti-PspA antibody responses and increased the levels of protective immunity to challenge of vaccinated animals with a wild-type virulent S. pneumoniae strain (85). Representative results demonstrating that constitutive expression of the operons encoding the Saf and Stc fimbriae in antigen delivery vaccine vector strains are better at conferring protective immunity to pathogen challenge are presented in Table 6.

TABLE 6
Protective efficacy of PIESV strains expressing Saf or Stc
fimbriae in a constitutive manner and delivering the S. pneumoniae
PspA antigen to orally vaccinated BALB/c mice

| | Constitutively | No. of | |
|---|---|---|---|
| | expressed | mice alive/ | |
| Strain | fimbrial genes | total no. | % survival |
| χ11850(pYA4088) | saf | 10/19 | 52.6 |
| χ11851(pYA4088) | stc | 10/19 | 52.6 |
| χ9088(pYA4088) | none | 3/11 | 27.3 |
| None (PBS) (control) | — | 0/18 | 0.0 |

Seven-week-old BALB/c mice were immunized orally with ~1 × 10⁸ CFU of the indicated S. Typhimurium vaccine strains and boosted with the same dose 6 weeks later. All mice were challenged by intraperitoneal inoculation 4 weeks after the booster dose with ~1 × 10⁴ CFU of virulent S. pneumoniae strain WU2. Deaths were recorded until 3 weeks post-infection.

It is also likely that the Saf and Stc fimbriae that represent MAMPs recruit innate immunity by interaction with some TLR, which has yet to be identified. Based on these observations, the addition of either or both the ΔP_{saf5}::P_{murA} safA and ΔP_{stc}::P_{murA} stcA deletion-insertion mutation to an improved PIESV vector strain should enhance colonization of the spleen to further enhance the level of induced protective immunity.

The Δ(agfG-agfC)-999 mutation eliminates the production of the proteinaceous thin aggregative fimbriae (also referred to as curli) that form a layer on the bacterial cell surface and can promote biofilm formation (84, 127-129). The dual agf operons are expressed both in vitro and in vivo (66). The AgfD gene product also serves as a regulator that activates genes for the production and secretion of cellulose (104) and the LPS O-antigen capsule (91) to result in biofilm formation. Although the Δ(wza-wcaM)-8 mutation present in the parent PIESV vector strains listed in Tables 3, 4 and 5 effectively precludes synthesis of polysaccharide capsular materials that can form biofilms, the addition of the Δ(agfG-agfC)-999 mutation would eliminate a different means to accomplish this objective of precluding biofilm formation. Wild-type S. typhimurium with the Δ(agfG-agfC)-999 mutation (χ11351) has the same LD₅₀ as the UK-1 parent and colonizes internal lymphoid tissues such as the spleen to the same titers as χ3761 prior to the onset of death of infected BALB/c mice. The inability to make biofilms also facilitates the completeness of the regulated cell lysis due to the chromosomal ΔP_{murA25}::TT araC P_{araBAD} murA ΔasdA27::TT araC P_{araBAD} c2 mutations and the presence of a regulated lysis plasmid with asdA and murA gene expression dependent on arabinose that is unavailable in vivo (FIG. 1) (17). Furthermore, the production of biofilm components that cover the PIESV cell surface inhibit immunological surveillance of antigenic surface structures and outer membrane vesicles produced by the PIESV strain (76, 130). Thus, the addition of the Δ(agfG-agfC)-999 mutation to the improved PIESV vector strains is likely to further enhance their immunogenicity and further preclude their ability to survive to persist. This, in turn, should increase the induction of protective immunity against the Salmonella vector strain. Since Salmonella is one of the most common food borne pathogens causing diarrheal disease with some mortality, especially in infants, the elderly and the immunocompromised (131-136), inducing protective immunity against Salmonella infection is a very important added benefit of using PIESV vector technologies. Adding the Δ(agfG-agfC)-999 mutation to the improved PIESV strains can be easily achieved by use of the pYA4941 suicide vector listed in Table 1.

Although fully described below in terms of improved refinements, the regulated loss in ability of PIESV vectors to synthesize LPS O-antigen is key to providing a regulated delayed attenuation phenotype and with the added benefit of exposing the PIESV cell surface for better immunosurveillance to result in enhanced immune responses to Salmonella surface antigens and those produced and secreted by the PIESV strain. This phenotype is thus beneficial in conjunction with means to eliminate production of capsular materials to cover the PIESV cell surface, and both contribute to enhanced induction of protective immunity to Salmonella infection.

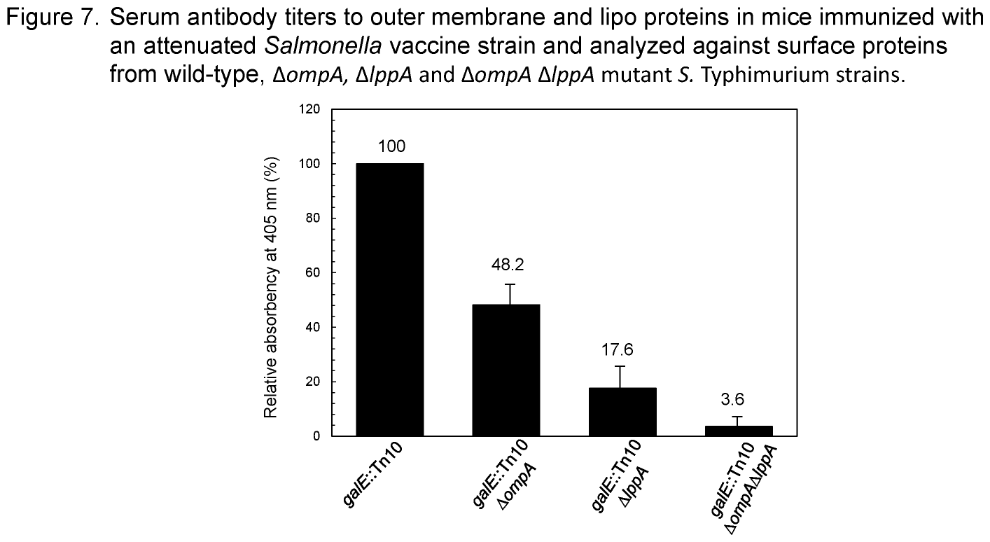

The OmpA outer membrane protein and the Lpp lipoprotein are two of the most prevalent proteins in the Salmonella outer membrane (86, 137-139). In mice immunized with a Salmonella vaccine strain attenuated by inactivating the galE gene to eliminate synthesis of the LPS 0-antigen and outer core, the antibody responses to these two proteins constitute over half of the serum antibodies to the Salmonella cell surface (FIG. 7). While deletion of the lpp genes encoding synthesis of Lpp reduces virulence (139), the ompA gene can be deleted from the wild-type UK-1 parent χ3761 without impairing Salmonella growth or virulence with an LD₅₀ of ˜6×10⁴ CFU for orally inoculated BALB/c mice. In this regard, while the OmpA protein plays no role in S. typhimurium adherence to or invasion into cells in culture, it nevertheless, is very immunogenic (140-144), induces DC maturation via TLR4, p38 and ERK1/2 activation and further enhances Th1 polarization of the immune response (140, 145). The purified S.

Typhimurium OmpA protein parenterally administered to mice also induces DC maturation and profound activation of MHC II molecules to induce CD4 and CD8 specific responses (145, 146) However, in addition to not decreasing virulence by deletion of the ompA gene, it appears that this deletion does not decrease the immunogenicity of Salmonella vaccine strains and does not reduce the level of protective immunity induced against challenge of immunized mice with wild-type virulent Salmonella. In these regards, the OmpA protein is a subterfuge since it induces high titers of antibodies that do not contribute to protective immunity against Salmonella. In fact, immunizing with an attenuated ΔompA11 Salmonella vaccine induces higher titers of antibodies to other outer membrane proteins that likely contribute to protective immunity since deletion of combinations of the ompC, ompF, ompD and ompW genes encoding other significant outer membrane proteins can reduce virulence (147-149). In this regard, while single ompF and ompC deletion mutants are virulent, mutants deficient in both ompC and ompF are attenuated (147). Results with ompD mutants are mixed showing decrease in virulence (148), increase in virulence (149) and no effect on virulence (150). Although deletion of ompW does not alter virulence (149), it is likely that multiple deletion mutations to eliminate synthesis of multiple outer membrane proteins will collectively decrease fitness and virulence. The fact that the OmpA protein can be eliminated with no detriment and possibly a benefit enables the modification of the OmpA protein by deletion of parts encoding surface-exposed domains and replacing these with segments of proteins from other pathogens capable of inducing protective immunity to that donor pathogen. Alternatively, the OmpA protein can be replaced by insertion of a sequence encoding an OMP from some other pathogen. This provides a very efficient and effective means because of the high prevalence and high immunogenicity of OmpA to deliver pathogen antigens conferring protective immunity.

Another attribute that can be added to a PIESV vector construct is a mutation resulting in constitutive expression of the spv operon on the Salmonella virulence plasmid (designated pSTUK100) in the S. typhimurium UK-1 strains). The spvR gene encodes a regulatory protein that self regulates its own expression and controls expression of the adjacent virulence plasmid spvABCDE operon (151-155) that governs the ability of S. typhimurium strains to traffic from the intestinal tract to internal effector lymphoid tissues such as the spleen (156, 157). Mutations in the promoter regions of the spvA-E operon can enable over expression of the spvA-E operon to increase the ability of PIESV constructs to colonize the spleen and other lymphoid tissues more efficiently. Alternatively, the spvABCD operon can be inserted into the chromosome to be expressed under the control of a constitutive promoter such as that for the cysG gene (that is unessential for virulence) or a mutant Pₛₚᵥ promoter no longer dependent on activation by SpvR or in which the spvR gene on the pSTUK100 virulence plasmid has been deleted or inactivated. We thus constructed the strain χ9877 ΔcysG175::Pₛₚᵥ spvABCD containing the pSTUK101 (in place of the wild-type pSTUK100) which has a ΔspvRABCD mutation that eliminates expression of the regulatory SpvR protein. In comparison to the wild-type S. typhimurium strain χ3761, χ9877 colonized spleen and liver of BALB/c mice to about one log higher titers 3 and 6 days after oral inoculation.

In previous studies (60), we used two different balanced-lethal host-vector systems in the same vaccine construct to enable encoding and delivering multiple protective antigens. Thus, in addition to using Asd⁺ vectors in a vaccine vector strain with a ΔasdA mutation, the vaccine strain possessed the Δalr-3 and ΔdadB4 mutations eliminating synthesis of the two alanine racemases made by Salmonella. This enabled use of a plasmid vector with the DadB⁺ selective marker to encode these additional protective antigens. In this regard, DadB⁺ plasmid vectors are available with different copy numbers using different ori sequences. pYA4346 has the p15A ori, pYA4015, p4554 and pYA4635 have the pBR ori, and pYA4552 has the pUC ori (60). All of these vectors have the DadB⁺ selective marker plus a multi-cloning site followed by a transcription terminator but have different promoters other than P_{trc} for regulating expression of inserted DNA sequences encoding protective antigens. To enable use of these DadB⁺ plasmids one can modify PIESV vector strains by introducing the Δalr-3 and ΔdadB4 mutations (Table 2) by using the suicide vectors described in Table 1. We also have a series of plasmid vectors with differing copy numbers that have ΔroA⁺, ΔroC⁺ and ΔroD⁺ selective markers for use in PIESV strains with attenuating ΔaroA, ΔaroC and ΔaroD mutations.

### Example 8. Modulation in the Rate of Display of Regulated Delayed Attenuation Attributes Relative to Timing of Vaccine Cell Persistence and Display of LPS O-Antigen Prior to Lysis

The PIESV strains listed in Tables 3, 4 and 5 all possess genes (murA, lacI in Family 1 and 2 strains and fur and (sometimes) mntR in Family 2 strains) whose expression is dependent on the presence of the sugar arabinose that can be supplied during growth of the vaccine strains prior to use for vaccination and which is absent in vivo. They also have the waaL gene whose expression is necessary for attachment of the LPS O-antigen components to the LPS core polysaccharide dependent of the presence of the sugar rhamnose during growth of the vaccine strains prior to use for vaccination and which is also absent in vivo. Salmonella actively transports arabinose and rhamnose such that at the time of harvesting vaccine cells, both arabinose and rhamnose exist within cells to enable continued expression of genes controlled by the araC P_{araBAD} and rhaRS P_{rhaBAD} cassettes. However, such expression is of very short duration since the vaccine cells rapidly catabolize the sugars to be used as energy sources with production of CO₂, H₂O and some acids during the process of being introduced into a vaccinated host. By preventing such catabolism of arabinose and rhamnose it can be predicted that the arabinose and rhamnose will be retained within vaccine cells with continued synthesis of arabinose- and rhamnose-regulated genes until the concentrations are sufficiently diluted by cell division or are leaked out of the vaccine cells. To evaluate these concepts the ΔaraBAD65::TT and ΔrhaBADSR515 mutations that delete all the genes necessary for the catabolism of arabinose and rhamnose were deleted in the Family 1 and 2 strains. The expected consequences of introducing these mutations will lead to the delayed time of lysis when strains with one of the regulated lysis plasmids (FIG. 1) are transferred from Purple broth growth medium with arabinose and rhamnose to Purple broth without the two sugars and also to a decreased rate of in vivo loss in the display of the LPS O-antigen of the PIESV cell surface. Results of experiments validating these expectations are presented below for strains after inclusion of another alteration to further delay the in vivo loss in the display of the LPS O-antigen.

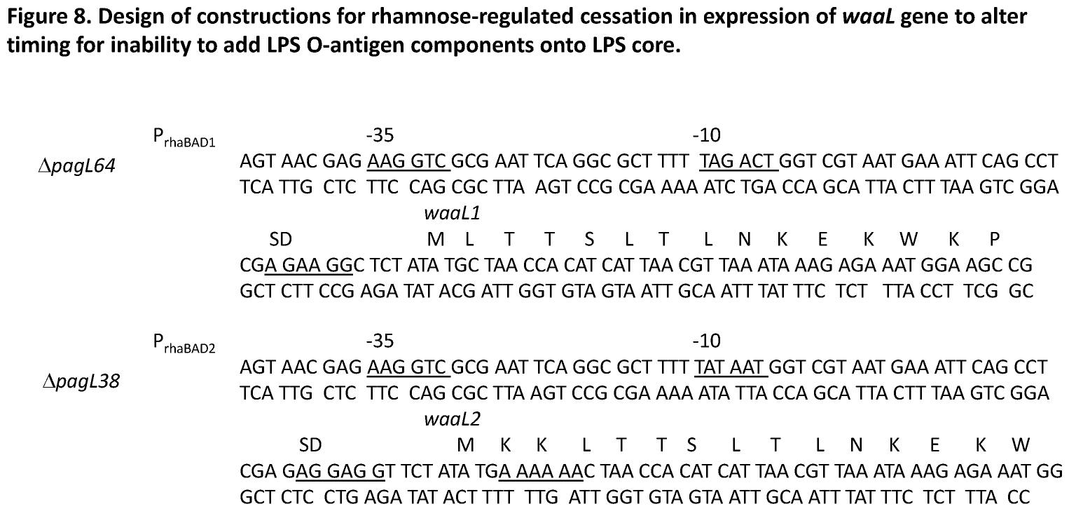

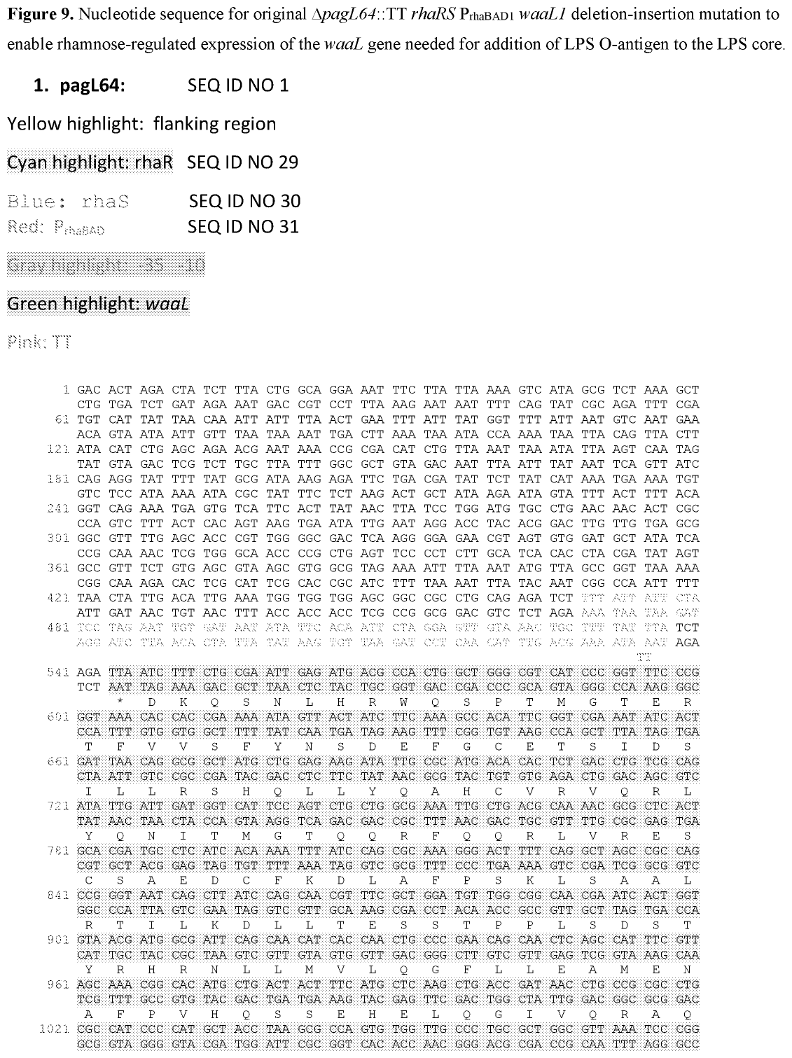

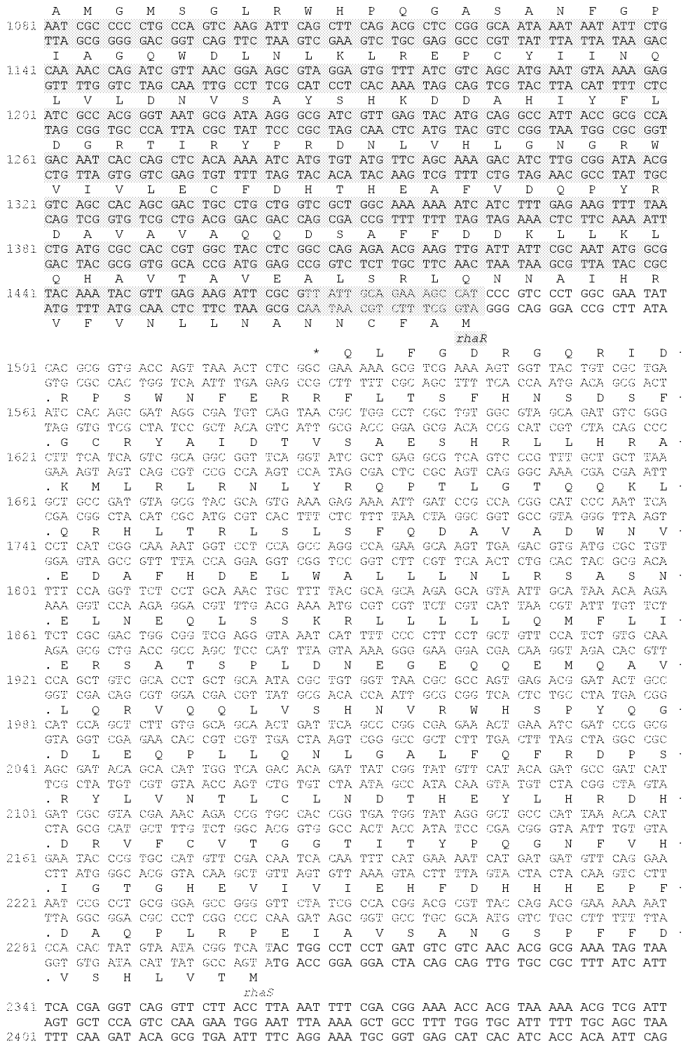

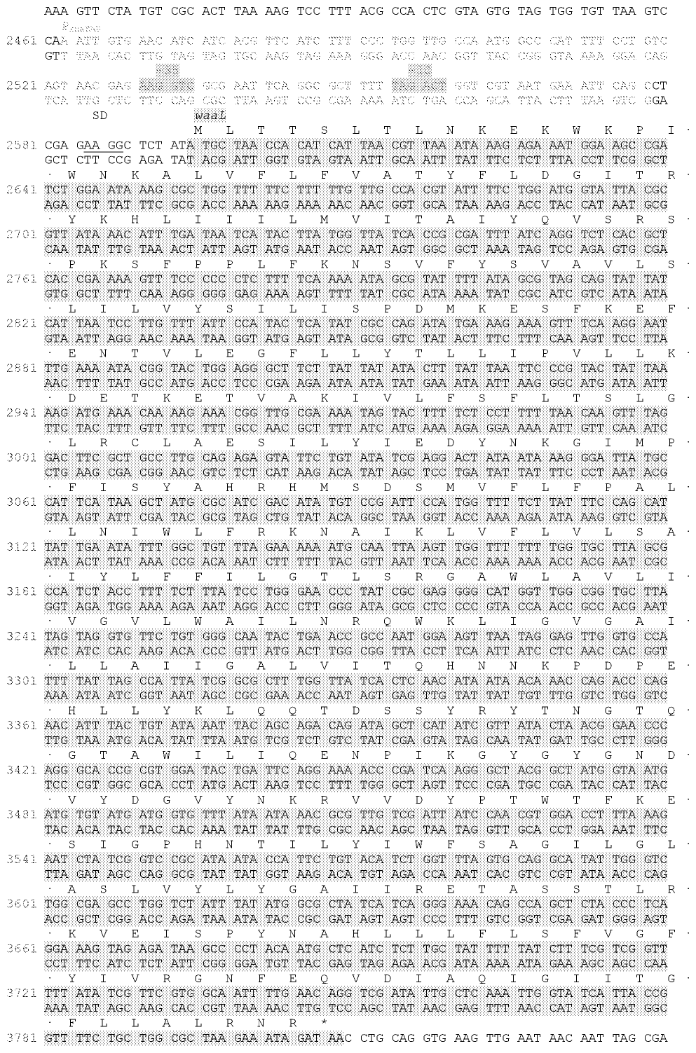

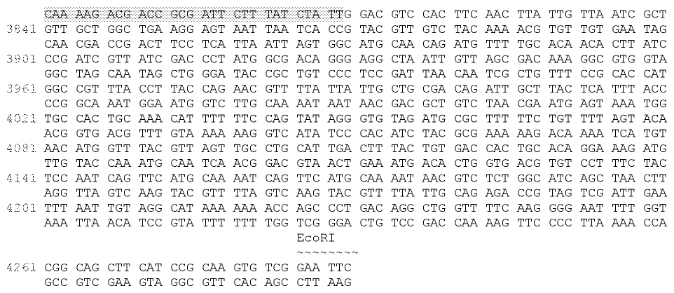

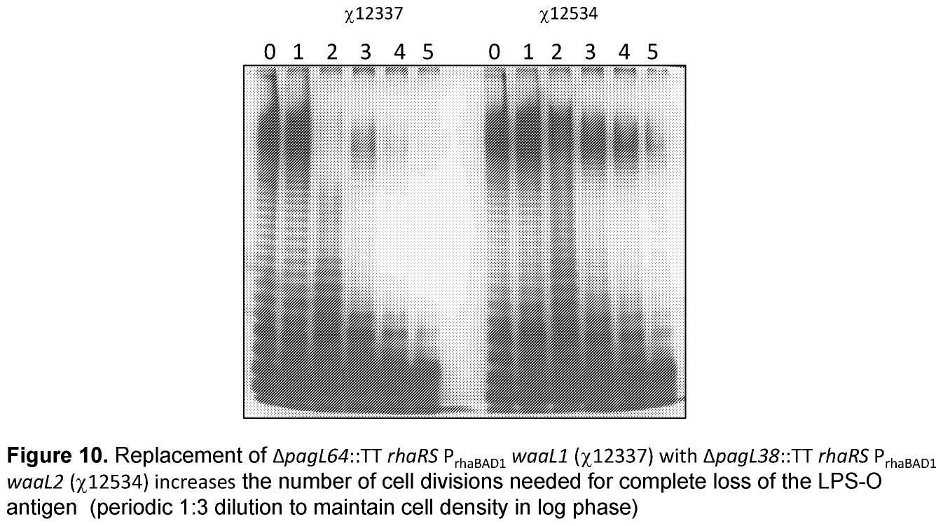

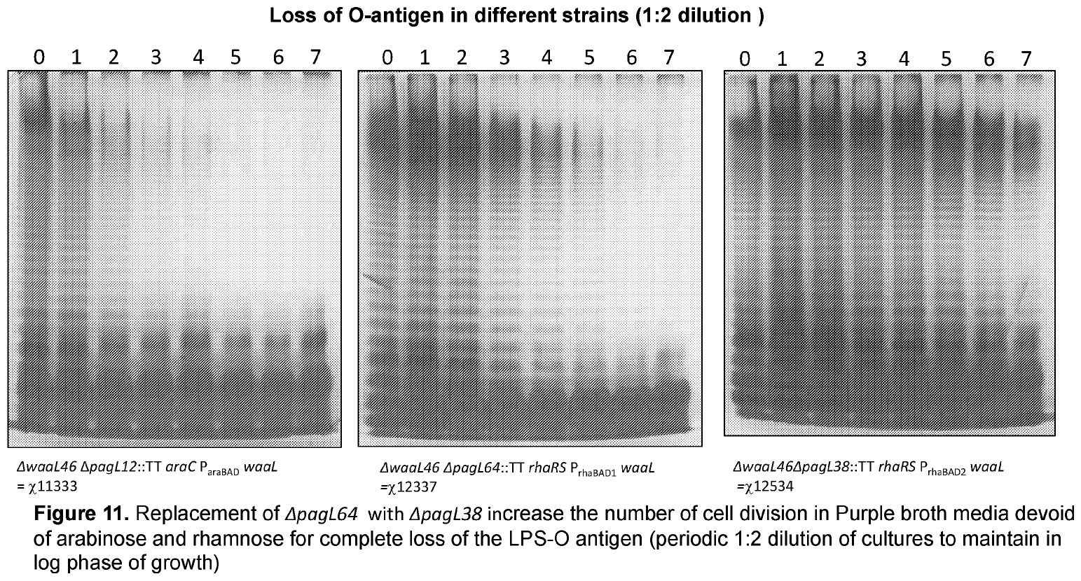

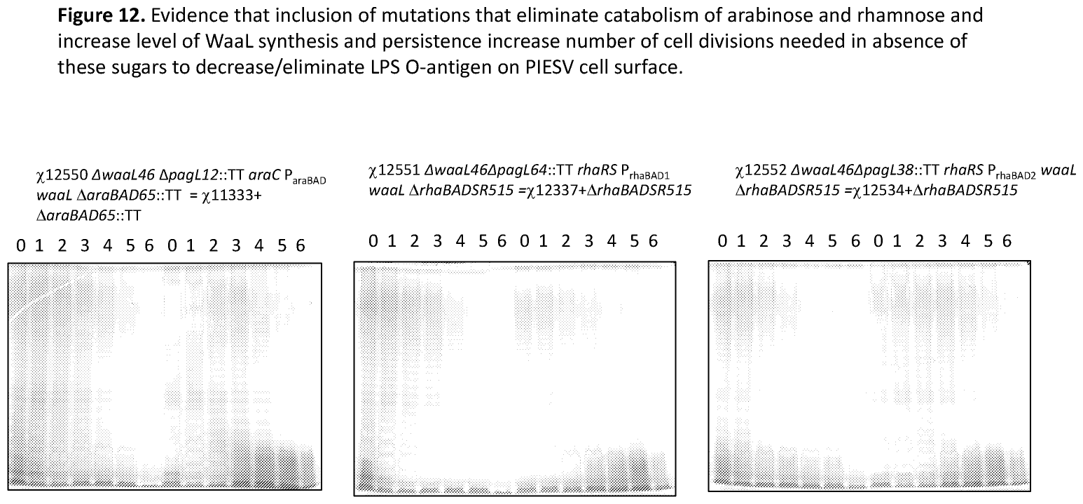

The attenuation resulting from the regulated delayed loss of LPS O-antigen from the surface of PIESV cells in vivo is a composite of increased susceptibility to phagocytosis (158-161) by a variety of cells with phagocytic capabilities and to an increased sensitivity to complement-mediated cytotoxicity (160, 161). While phagocytosis probably enhances induction of immune responses due to antigen processing and presentation in degrading PIESV cells, the sensitivity to complement is due to complement-mediated destruction of lipid bilayers that effectively kills cells very rapidly. In using bacterial vectored vaccines, the temporal sequence of events is of critical importance. Thus, PIESV cells must effectively colonize internal effector lymphoid tissues, invade into cells in those tissues including phagocytic and dendritic cells, synthesize and deliver protective antigens, display attenuation so as not to induce disease symptoms and undergo regulated delayed lysis to release synthesized protective antigens or DNA vaccines encoding them. The complete loss of LPS O-antigen that would result in PIESV cell killing by complement should thus be delayed since once PIESV cells are dead, they can no longer synthesize protective antigens or grow to lyse to liberate these or a DNA vaccine. To achieve this desirable timing of programmed events in the life and function of PIESV cells, the level of synthesis of the WaaL protein was increased so that it would take several more cell divisions to dilute its concentration to essentially cease adding LPS O-antigens side chains to the PIESV cell surface. This can be achieved in several ways such as by changing the −10 RNA polymerase binding domain within the P_{rhaBAD} promoter, by increasing the number of base pairs within the Shine-Dalgarno (SD) ribosome binding domain upstream of the ATG start codon for the waaL gene that are complementary to sequences in the 16S ribosomal RNA (which increases ribosome loading on the mRNA to increase translation levels of the waaL mRNA) and/or by inserting AAA codons as the 2^{nd} and 3rd codons in the waaL gene that also significantly increases translation of the waaL mRNA. We thus designed a suicide vector pG8R296 (Table 1) to enable the replacement of the ΔpagL64::TT rhaRS P_{rhaBAD1} waaL1 with the ΔpagL38::TT rhaRS P_{rhaBAD2} waaL2 sequence. The critical sequence information for these two constructions are depicted in FIG. 8 in which the P_{rhaBAD}-10 sequence was changed from TAGACT to TATAAT, the SD sequence changed from AAGG to AGGA and two Lys AAA codons inserted after the ATG start codon in the ΔpagL38::TT rhaRS P_{rhaBAD2} waaL2. The nucleotide sequence for the original ΔpagL64::TT rhaRS P_{rhaBAD1} waaL deletion-insertion mutation is presented in FIG. 9. As indicated by the data in FIG. 10, this change increased the number of cell divisions for complete loss of the LPS O-antigen from the PIESV cell surface. Both χ12337 and χ12534 were derived from χ11312, which has the ΔwaaL46 mutation inserted into the wild-type S. typhimurium UK-1 strain χ3761. FIG. 11 presents data from another experiment comparing rates of losing display of LPS O-antigen when the waaL gene expression is under araC P_{araBAD} control (χ11333 also derived from χ11312) versus the two constructs χ12337 and χ12534 with altered rhaRS P_{rhaBAD} control of waaL expression. that also compares enhanced delay by inclusion of the ΔaraBAD65::TT mutation in χ12550, which was derived from χ11333 and inclusion of the ΔrhaBADSR515 mutation in χ12551 and χ12552. FIG. 12 presents data to show that this delay in loss of LPS O-antigen on cell surface is delayed further by inclusion of ΔaraBAD65::TT and ΔrhaBADSR515 mutations to block catabolism or arabinose or rhamnose present in vaccine cells at the time of harvesting from the fermenter.

For this experiment, χ12550 was constructed by introducing the ΔaraBAD65::TT mutation into χ11333 and χ12551 and χ12552 by introducing the ΔrhaBADSR515 mutation into χ12337 and χ12534. It should be noted that this delay in LPS O-antigen loss due to inclusion of the ΔaraBAD65::TT and ΔrhaBADSR515 mutations and the increased level of WaaL synthesis and persistence delayed the timing for PIESV cell lysis and release of contents to enhance induction of immune responses against synthesized protective antigens or for the release of a DNA vaccine vector encoding such antigens.

### Example 9. Improved Regulated Delayed In Vivo Synthesis of Protective Antigens and Enhancement of Safety and Containment of PIESV Strains

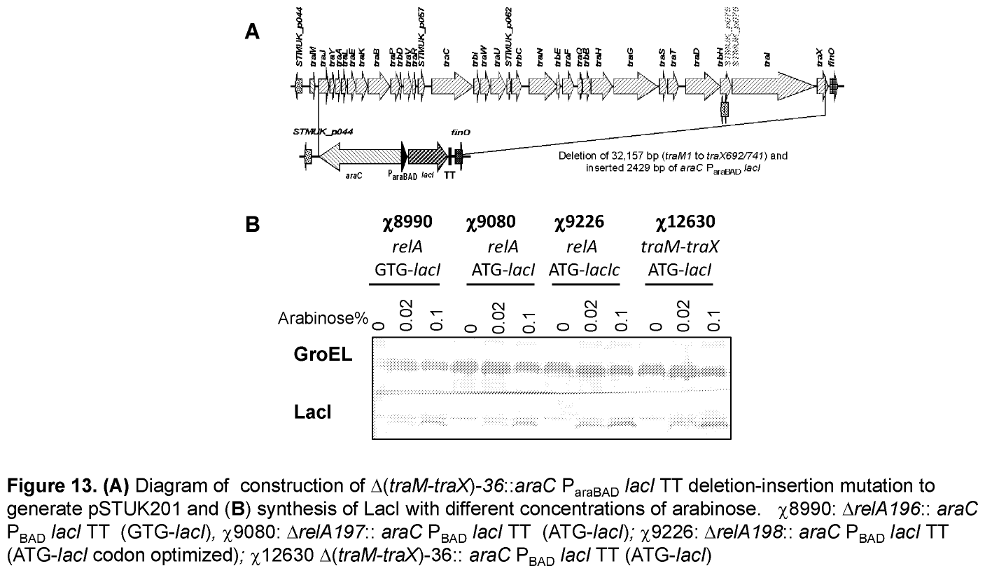

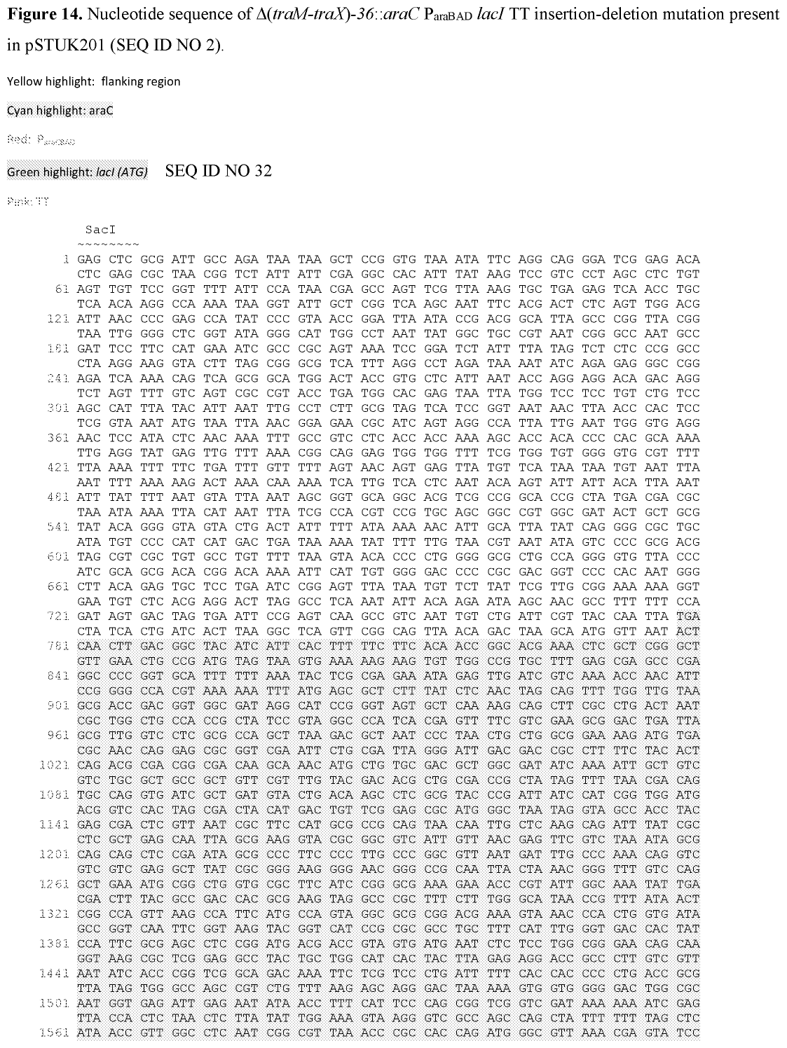

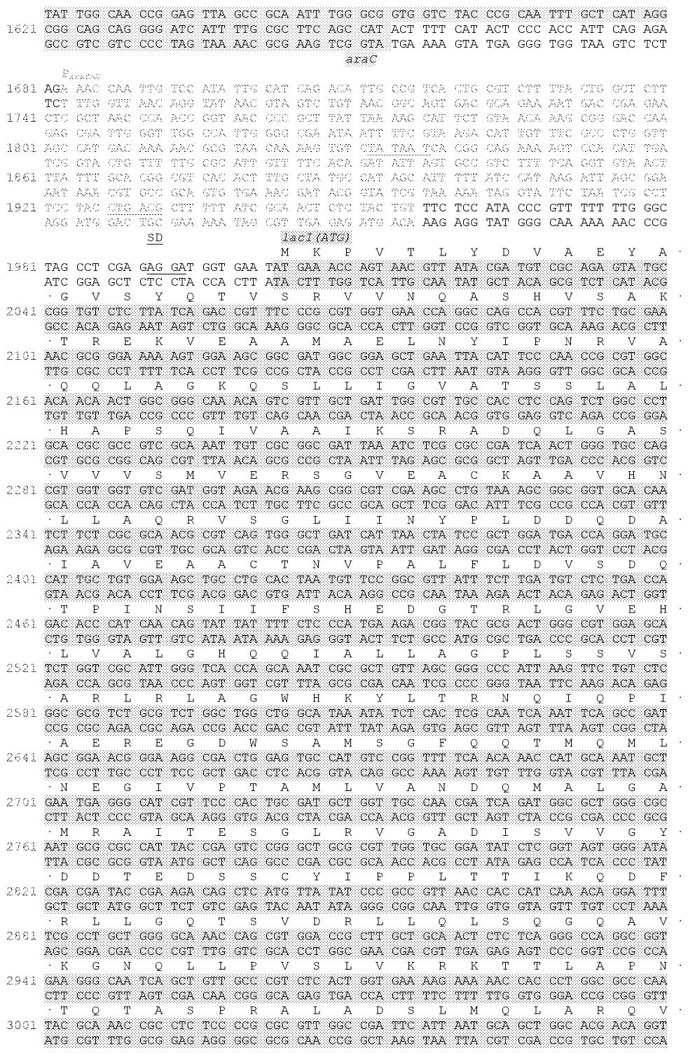

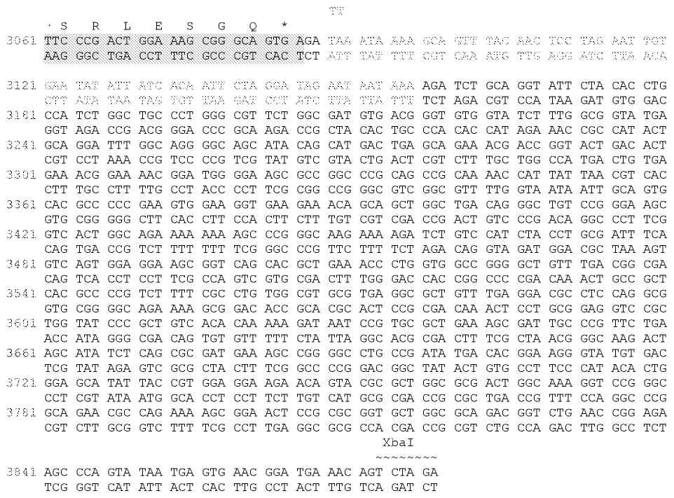

Since synthesis of protective antigens encoded on regulated delayed lysis plasmid vectors represents a metabolic burden to PIESV cells, which in turn decreases growth rate, colonizing ability and ultimately the magnitude of induced immune responses, we devised a regulated delayed means for in vivo synthesis of protective antigens so that maximal synthesis would be achieved after colonization of internal effector lymphoid tissues (16). This was achieved by inserting an araC P_{araBAD} cassette fused to the lacI gene encoding a repressor that blocks synthesis of genes under the control of a promoter such as P_{trc} that possess the lacO sequence to which LacI binds. The regulated delayed lysis plasmids diagrammed in FIG. 1 all have a cloning site downstream from P_{trc} The timing of derepression for the expression of plasmid encoded genes for protective antigens was varied by changing the −10 sequence in P_{araBAD}, the SD sequence, the spacing between the SD sequence and the start codon and the sequence of the start codon (16). Each of these types of changes would alter the level of LacI synthesized during PIESV growth and would thus require different numbers of cell divisions in vivo to achieve maximal levels or protective antigen synthesis. These different araC P_{araBAD} lacI TT cassettes were inserted into deletions of the relA gene that uncouples growth from a dependence on protein synthesis (facilitates complete lysis of PIESV strains) and the endA gene that eliminates a periplasmic endonuclease (16). The maximum amounts of LacI synthesized by the constructs designed to produce three levels of LacI synthesis are presented in FIG. 13B. Since the relA gene can be beneficially deleted in PIESV strains and is a permissive site for the insertion of heterologous genes specifying gene products that could be protective antigens or serve other beneficial attributes to the PIESV strains, we considered plasmid display of the sugar-regulated synthesis of the LacI repressor. The S. typhimurium virulence plasmid is a low-copy number 90 kb plasmid that possesses genetic information to enable conjugation with other bacteria (162, 163) (in addition to the spv operon that enhances colonization of the spleen) that could lead to transfer of the virulence plasmid or by mobilization other plasmids present in the same recombinant PIESV cell into other bacteria present in the vaccinated host or environment. Also, there is some evidence that there is a total amount of plasmid DNA that can be maintained per bacterial cell. Since gene activity in bacteria is influenced by gene copy number, the reduction in the size of the virulence plasmid would potentially increase the copy number of the regulated lysis plasmids designed to specify synthesis and delivery of protective antigens from various pathogens. This could thus give a slight boast to the induced antibody responses since the magnitude of immune responses is related to the amount of antigen delivered (159). Taking all these points into consideration, an araC P_{araBAD} lacI TT cassette was inserted into a 32,157 bp deletion of the traM to traX genes in pSTUK100 using a suicide vector delivering the Δ(traM-traX)-36::araC P_{araBAD} lacI TT insertion to generate pSTUK201 (FIG. 13A). Western blot data comparing the maximum levels of LacI synthesized depending on arabinose concentration are presented in FIG. 13B for strains derived from the wild-type S. typhimurium UK-1 χ3671 strain with the chromosomal and virulence plasmid sites for the araC P_{araBAD} lacI TT sequences. It should be noted that a TT sequence is placed after the C-terminal end of the lacI gene to preclude transcription of sequences downstream from the lacI insertion. FIG. 14 provides the DNA sequence of the Δ(traM-traX)-36::araC P_{araBAD} lacI TT insertion and the flanking genes. The Family 1 and 2 strains with this pSTUK201 display regulated expression of genes encoded on lysis plasmids that can be derepressed by addition of the inducer IPTG since elimination of arabinose (that leads to derepression in vivo) would lead to cell lysis. It should be noted that the replacement of the chromosomal ΔrelA197::araC P_{araBAD} lacI TT mutation by addition of pSTUK201 necessitate the introduction of the chromosomal ΔrelA1123 to ensure the maintenance of the RelA phenotype to uncouple growth from a dependance on protein synthesis to ensure maximal regulated delayed lysis.

The level of the lacI gene product resulting from growth of PIESV strains in media with 0.1% arabinose will determine the number of cell divisions in vivo after inoculation of the PIESV strain into an animal host when derepression of antigen-encoding genes regulated by P_{trc} commence to be expressed. We can thus choose a construction analogous to those in the strains with the ΔrelA196, ΔrelA197 and ΔrelA198 alleles that specify low, moderate and high LacI levels (FIG. 13B). We can thus chose to modify the Δ(traM-traX)-36::araC P_{araBAD} lacI TT construction in pSTUK201 to specify low, moderate and high LacI levels. A low level of LacI is desirable if synthesis of the protective antigen encoded by the P_{trc}-regulated gene does not significantly impair the growth of the PIESV strain and thus its ability to colonize internal effector lymphoid tissues in the vaccinated animal host. If, however, synthesis of the P_{trc}-regulated gene encodes a protective antigen that is somewhat toxic to the PIESV strain to impede growth and colonizing ability, it is preferable to use an arabinose-regulated construct specifying a high level of LacI synthesis. These parameters are easily evaluated by comparing growth of PIESV constructs in media with 0.1% arabinose and 0.1% rhamnose but with and without IPTG to see the effect of antigen synthesis. Such modifications are readily made in derivatives of PIESV strains such as χ12615 and χ12616.

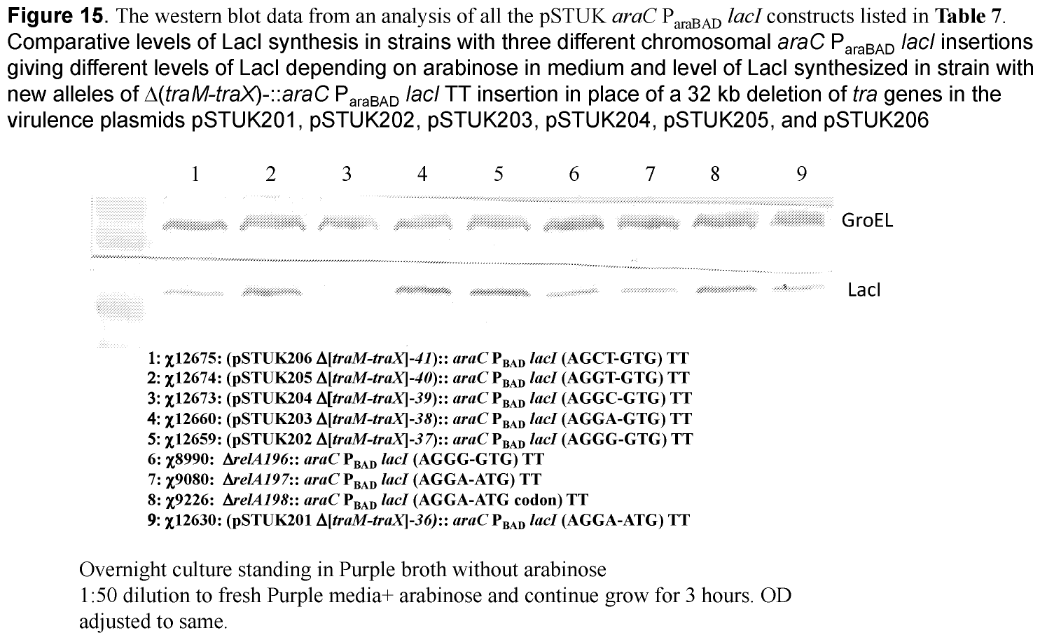

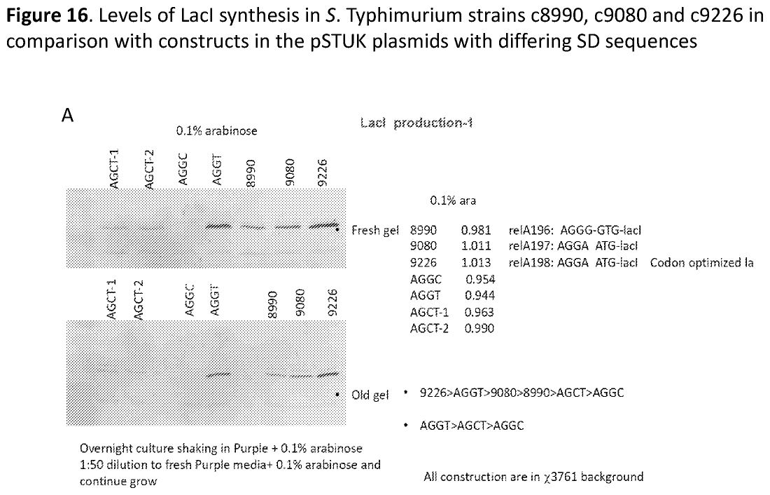

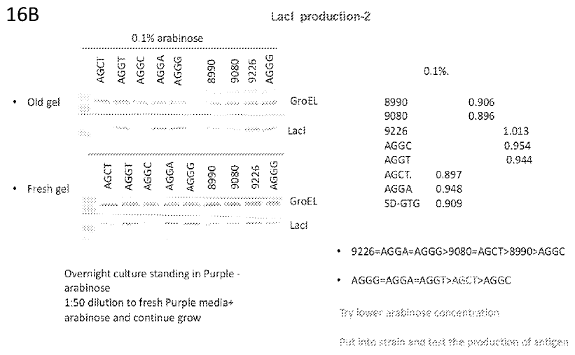

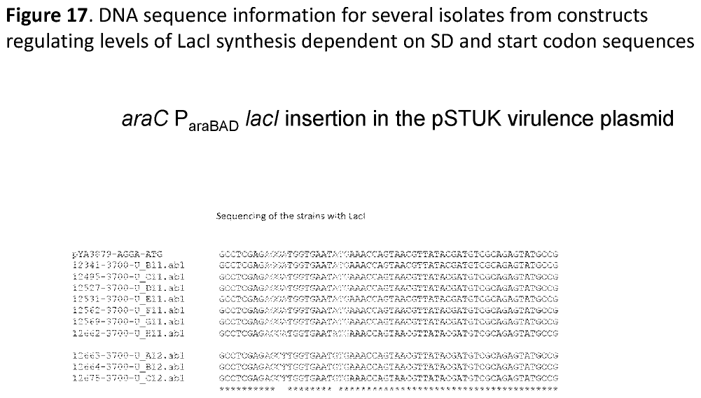

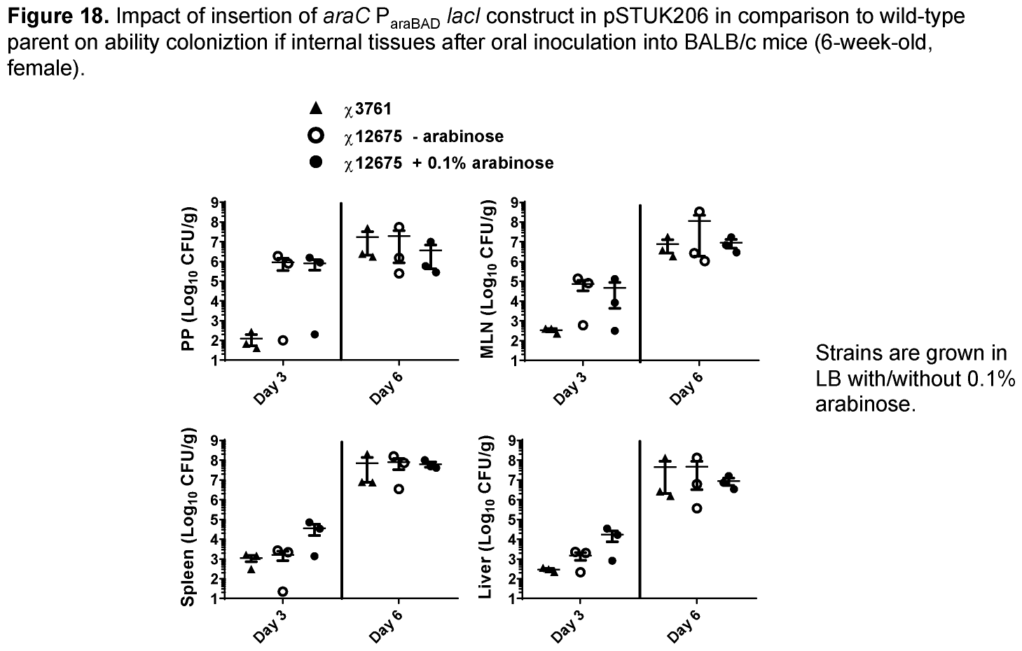

Subsequent comparative studies with synthesis of Streptococcus pneumoniae protective antigen PspA (164) in χ12615 with the pSTUK201 construct revealed that the level of LacI synthesized in LB broth with 0.1 percent arabinose caused a much-delayed synthesis of antigen analogous to what had previously observed in constructs with the ΔrelA198::araC P_{araBAD} lacI TT construction (see FIG. 13B). A series of modified constructs were thus made that would specify different levels of LacI synthesis during growth in LB broth with 0.1 percent arabinose due to changes in the SD sequence. The levels of LacI synthesis in these constructs in comparison to the levels of LacI made by the strains with the ΔrelA196, ΔrelA197 and ΔrelA198 alleles are shown in FIGS. 15A and B with the promoter region nucleotide sequences presented in FIG. 16. The levels of LacI synthesis made by the constructed strains with these sequences are presented in FIG. 17 and the properties of all these constructs are listed in Table 7. Since PIESV strains with the ΔrelA197::araC P_{araBAD} lacI TT deletion-insertion mutation gave programmed antigen gene expression optimally in regard to the timing of regulated expression of attenuation, cell wall and surface changes and lysis, we selected the Δ(traM-traX)-41::araC P_{araBAD} lacI TT construction in pSTUK206 to insert into strains to replace the originally inserted Δ(traM-traX)-36::araC P_{araBAD} lacI TT construction. This yielded χ12663 (Table 4) and χ12664 (Table 5). Additionally, the levels of LacI production in PIESV strains with pSTUK206 can be modified by using different arabinose concentrations for growth of the strains. The LD₅₀ data proved that the alterations in the virulence plasmid do not change the virulence of the strain (Table 8) or its ability to colonize internal tissues after oral inoculation (FIG. 18).

TABLE 7
araC ParaBAD lacl insertions in the pSTUK virulence plasmid.

| | | | | Sequence | |
|---|---|---|---|---|---|
| | | | | between SD | |
| | | | SD | and Start | Start |
| Strain | Genotype | Promoter | sequence | codon | codon |
| χ12630 | (pSTUK201 Δ[traM- | araC P_{BAD} | AGGA | TGGTGAAT | ATG |
| | traX]-36)::araC P_{BAD} | | | | |
| | lacI (AGGA-ATG) | | | | |
| | TT | | | | |
| χ12660 | (pSTUK203 Δ[traM- | | AGGA | | GTG |
| | traX]-38)::araC P_{BAD} | | | | |
| | lacI (AGGA-GTG) TT | | | | |
| χ12659 | (pSTUK202 Δ[traM- | AGGG | GTG | | |
| | traX]-37)::araC P_{BAD} | | | | |
| | lacI (AGGG-GTG) | | | | |
| | TT | | | | |
| χ12673 | (pSTUK204 Δ[traM- | AGGC | GTG | | |
| | traX]-39::araC P_{BAD} | | | | |
| | lacI (AGGC-GTG) TT | | | | |
| χ12674 | (pSTUK205 Δ[traM- | AGGT | GTG | | |
| | traX]-40):: araC P_{BAD} | | | | |
| | lacI (AGGT-GTG) TT | | | | |
| χ12675 | (pSTUK206 Δ[traM- | AGCT | GTG | | |
| | traX]-41)::araC P_{BAD} | | | | |
| | lacI (AGCT-GTG) TT | | | | |

•All strains are derived from S. Typhimurium χ3761

TABLE 8
The LD₅₀ of χ12675 in female 6-week-
old BALB/c mice by oral immunization

| | | # of Survival | |
|---|---|---|---|
| | | mice/Total | Mean Day |
| Strains | Dose | number | of Death |
| X3761 | 1.5 × 10³ | 3/3 | — |
| | 1.5 × 10⁴ | 3/3 | — |
| | 1.5 × 10⁵ | 0/3 | 9.7 |
| X12675 | 1.3 × 10³ | 3/3 | — |
| | 1.3 × 10⁴ | 3/3 | — |
| | 1.3 × 10⁵ | 1/3 | 7 |
| X12675 | 1.2 × 10³ | 3/3 | — |
| 0.1% arabinose | 1.2 × 10⁴ | 2/3 | 10 |
| | 1.2 × 10⁵ | 2/3 | 7 |

All strains were grown in LB; 0.1% arabinose was added when needed

Three benefits are achieved by these improved means to achieve regulated delayed in vivo synthesis of protective antigens in (i) providing a freed-up permissive relA chromosomal site for gene insertion, (ii) enhancing the levels of induced immunities to plasmid-encoded protective antigens and (iii) enhancing biological containment by decreasing the probability for transfer of genetic information to other microorganisms beyond that which is achieved by the regulated delayed lysis in vivo phenotype.

### Example 10. Generation of a Regulated Delayed Lysis Plasmid Encoding Five Protective Antigens with Expression of Coding Sequences Regulated by Five LacI Regulated Promoters and with Four Antigens Secreted by Four Different Optimized Type 2 Secretion Systems

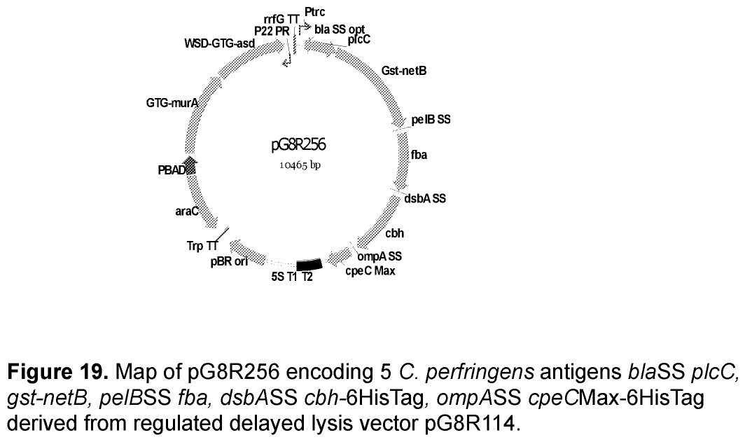

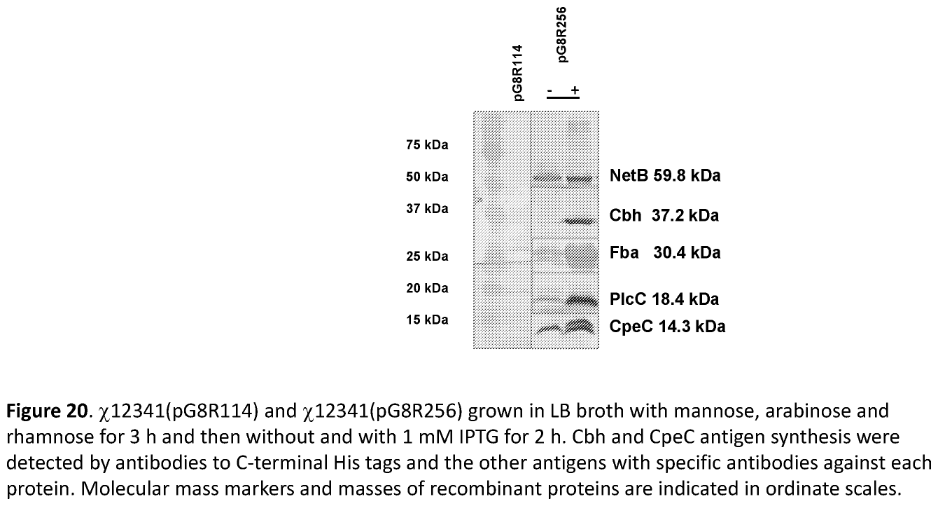

FIG. 19 diagrams the 10,465 bp regulated delayed lysis plasmid pG8R256 that specifies synthesis of 5 Clostridium perfringens protective antigens and FIG. 20 presents a western blot showing the IPTG-dependent synthesis of each of the five protective antigens by the triple sugar dependent PIESV strain χ12341, which is the parent of χ12495 (Tables 3 & 4). Antisera against the PlcC, Gst-NetB and Fba antigens were available and used whereas the Cbh and CpeC antigens were identified using an encoded C-terminal 6HisTag. The χ12341(pG8R256) vaccine construct is stable for 50 generations of growth in LB broth media with arabinose and DAP (permissive conditions) and continues to produce these five antigens at the end of this growth period. The plasmid is thus stably maintained and retains the ability to synthesize each of the five protective antigens.

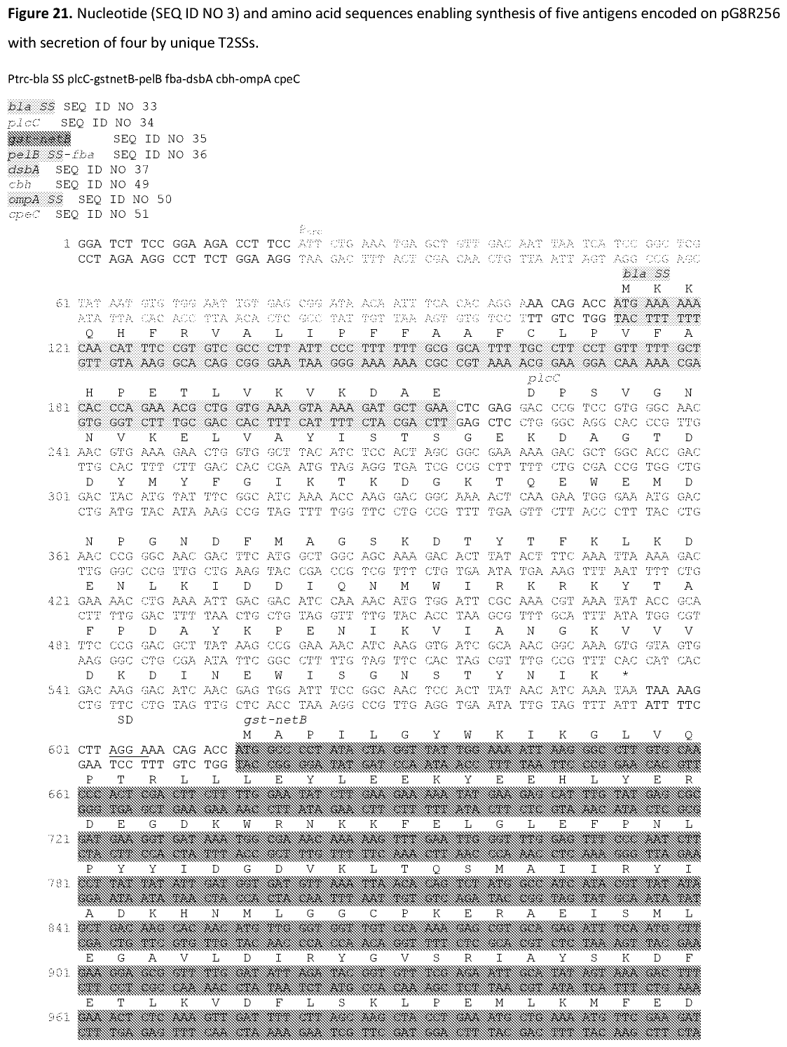

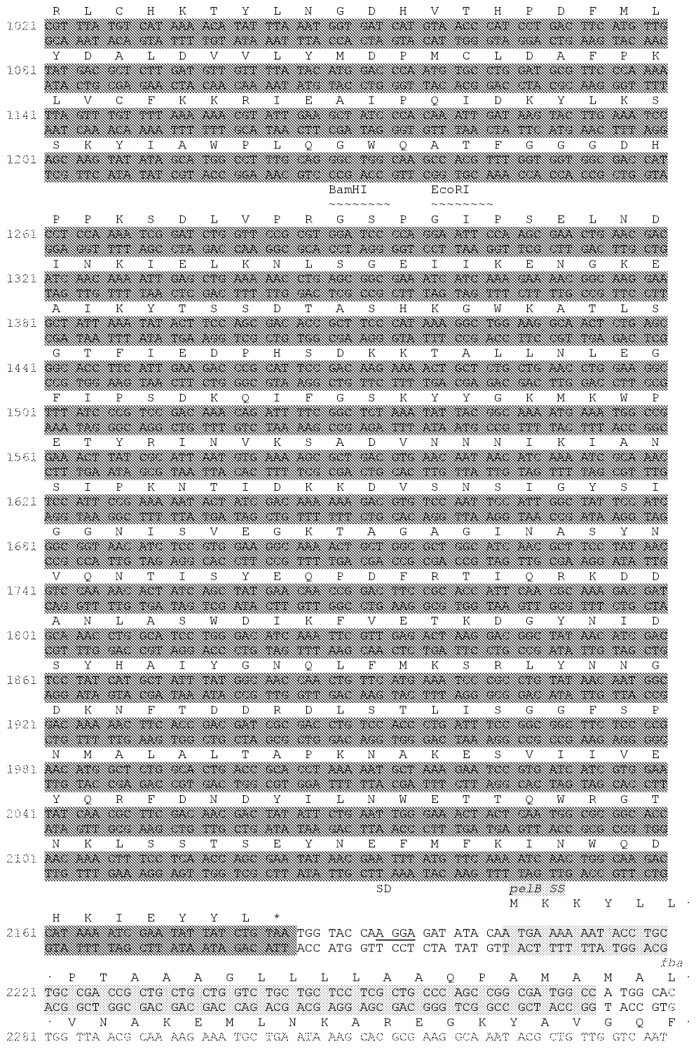

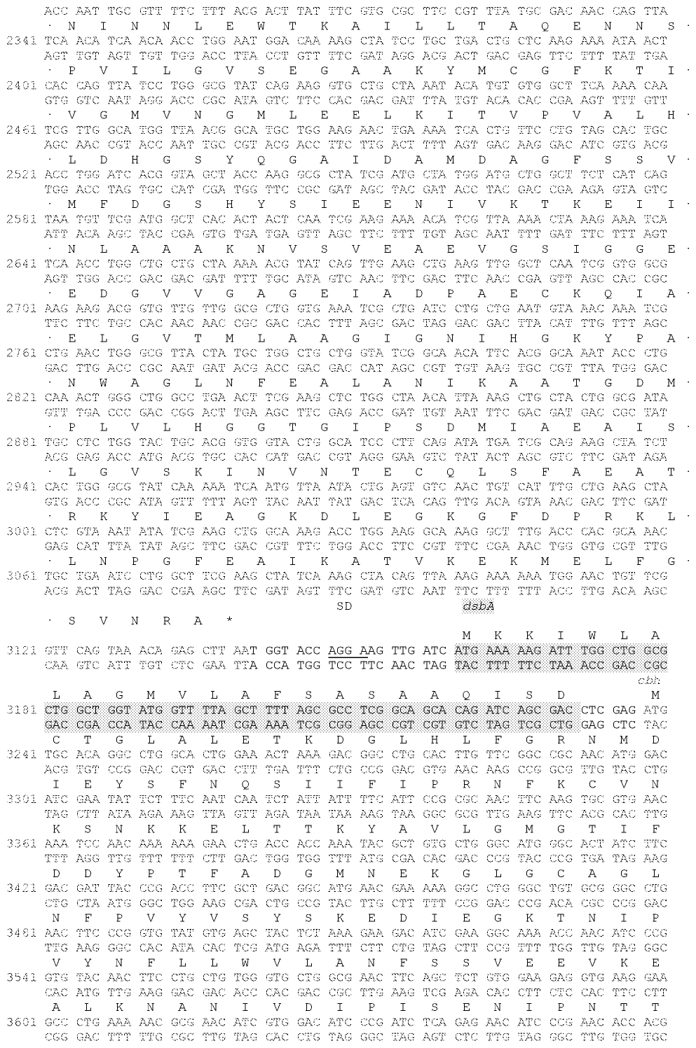

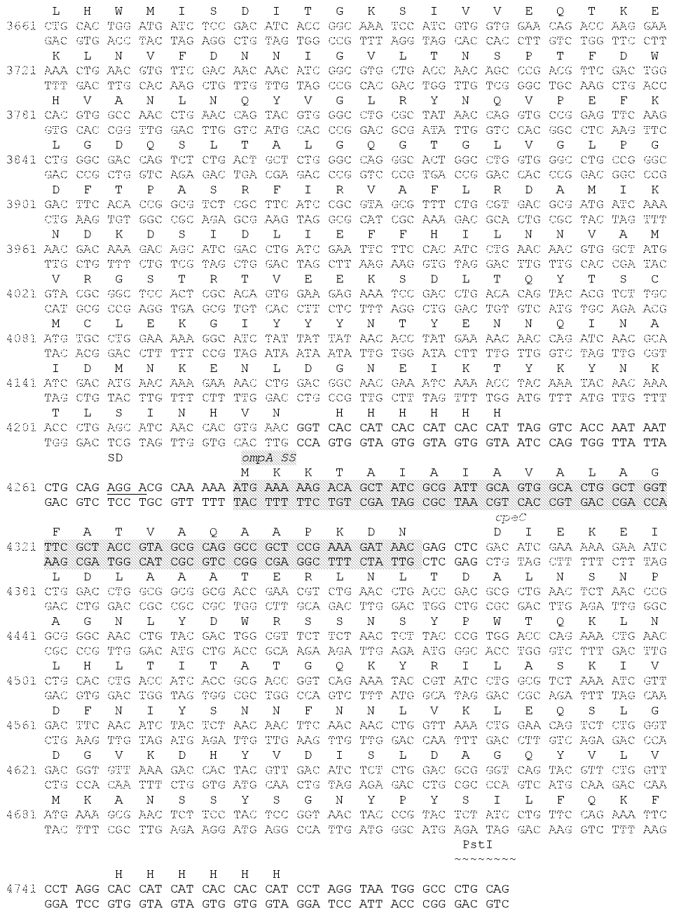

The plasmid pG8R256 (FIG. 19) encodes 5 protective antigens specified by the sequences blaSS plcC, gst-netB, pelBSS fba, dsbASS cbh-6HisTag, and ompASS cpeCMax-6HisTag that have all been codon optimized for high-level expression in Salmonella. All antigens have been individually proven to be protective (22, 165-167). Except Gst-NetB (which is toxic to vaccine cells if secreted), each antigen is fused to a unique Type II secretion signal. This will maximize immunogenicity and augment formation of outer membrane vesicles. FIG. 21 displays the nucleotide and amino acid sequences enabling synthesis and secretion of each of the four secreted antigens.

In regard to the derivation of pG8R256, its ancestor pYA3681 is a regulated delayed lysis in vivo plasmid originally described by Kong et al., (17). pYA3681 served as the backbone for the addition of an optimized blaSS described by Jiang et al. (21) resulting in plasmid pG8R114 (165, 168). The difference between the native blaSS and the optimized blaSS is the change of the second and third codons to AAA for Lys (see FIG. 19). Having A-rich codons in the second and third codon increases translation efficiency of mRNA significantly. This strategy was also used to optimize the pelBSS by insertion of AAA for Lys after the second codon (which is AAA already coding for Lys) of the native pelB secretion signal. Therefore, in one embodiment of the invention, a signal sequence can be a blaSS signal sequence or an optimized blaSS signal sequence. In one embodiment, the codon-optimized fba sequence can be inserted with an optimized pelBSS (see FIG. 19). pYA5130 is a pYA3681 derived plasmid encoding a native blaSS plcC-fba-gst-netB cassette. pG8R252 is a pG8R114 (see FIG. 1) derived lysis plasmid encoding 4 C. perfringens antigens with optimized blaSS-plcC, gst-netB, dsbASS-cbh-6HisTag, ompASS-cpeCMax-6HisTag. Using pYA5130 as a template (167), fba F1 was amplified with primers Pel/Fba-S2 (5′ GTCTGCTGCTCCTCGCTGCCCAGCCGGCGATGGCCATGGCACTGGTTAACGCAAAAG 3′) (SEQ ID NO 25) and Fba-KpnI-a (5′ GCGCGGTACCATTAAGCTCTGTTTACTGAACCGAAC 3′) (SEQ ID NO 26). The fba PF was amplified with template fba F1 using primers Pel-F2-S1 (5′ GATATACAATGAAAAAATACCTGCTGCCGACCGCTGCTGCTGGTCTGCTGCTCCTCG CTG 3′) (SEQ ID NO 27) and Fba-KpnI-a. The pelBSS-fba was amplified using primer Pel-F1-KpnI-S (5′ CGCGGGTACCAAGGAGATATACAATGAAAAAATACCTGC 3′) (SEQ ID NO 28) and Fba-KpnI-a with fba PF as a template. This pelBSS-fba fragment was cut with KpnI and cloned into pG8R252 to generate pG8R256. The sequence of pelBSS-fba was verified by sequencing and enzyme digestion.

### Example 11. Generation of a Regulated Delayed Lysis Plasmid Vector to Fuse Protective Antigens onto the Woodchuck Hepatitis Virus (WHV) Core with Specific Application to Make a Vaccine Against SARS-CoV-2

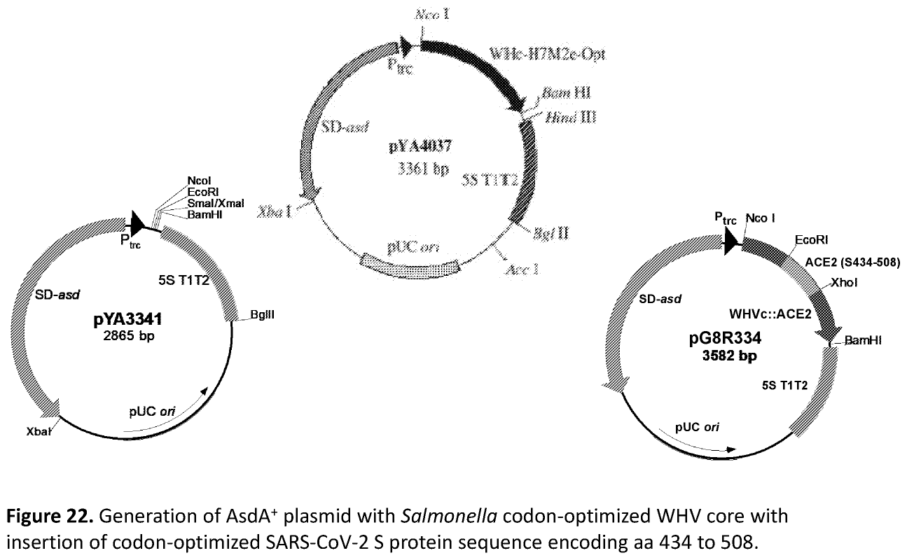

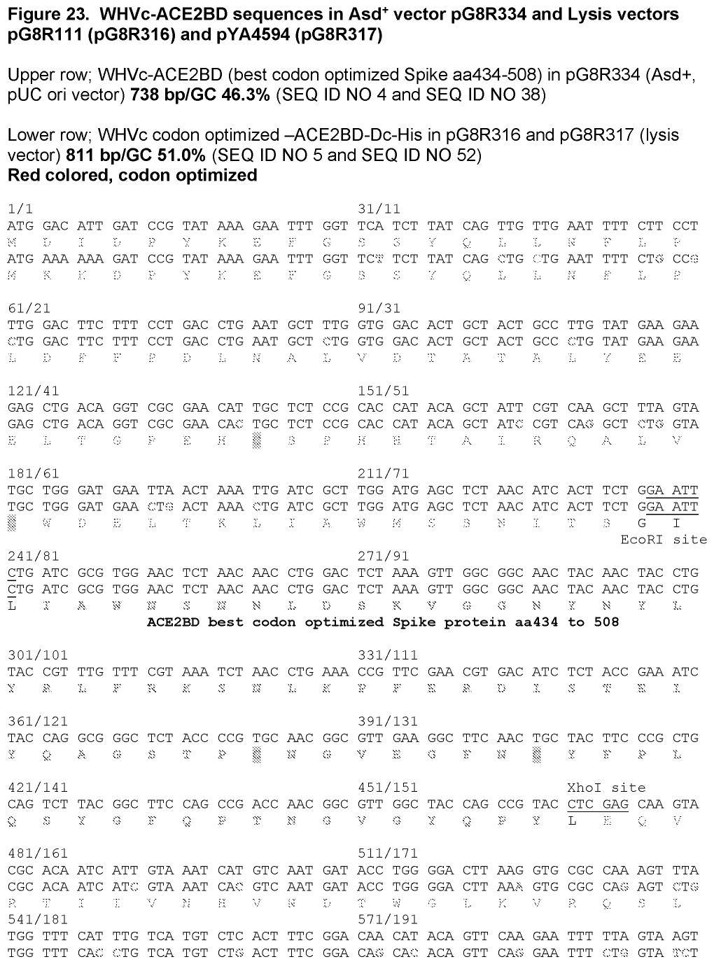

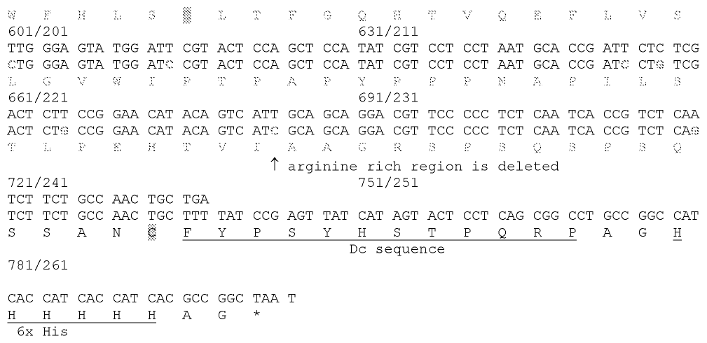

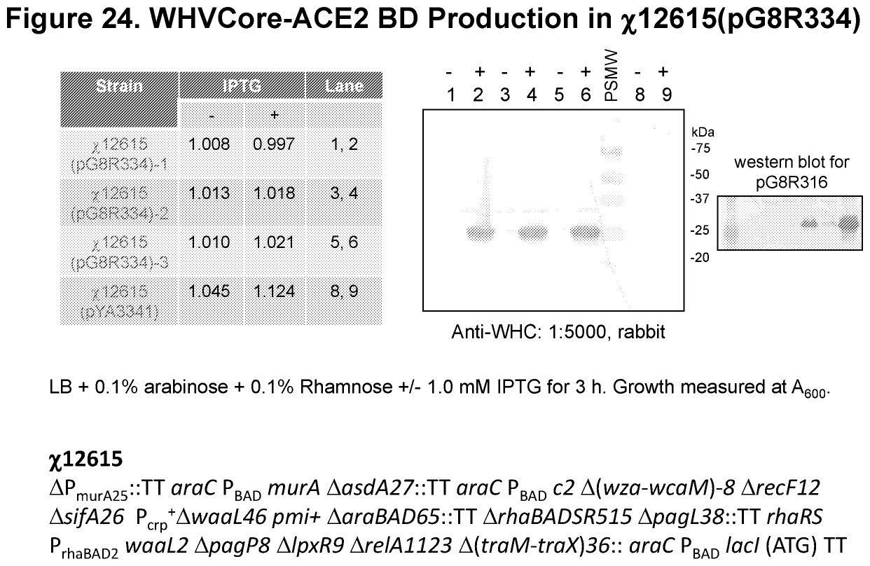

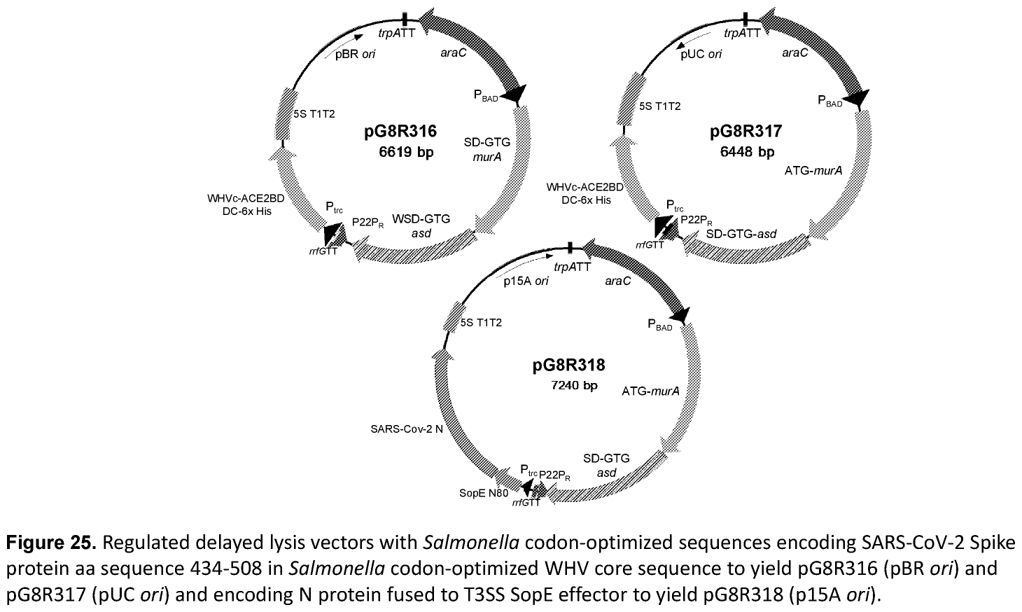

It is important to develop a diversity of vaccine vector platforms that can be rapidly deployed to design and develop vaccines against arising zoonotic, epidemic and pandemic pathogens. It is also important to have a diversity of means to deliver protective antigens to elicit desired types of immune responses. We diagramed three different regulated delayed lysis plasmid vectors in FIG. 1 that can be used to deliver protective antigens synthesized by the PIESV strain and released by lysis of the PIESV cells in vivo or in addition by secretion using either T2SS or T3SS. These vectors can be further modified as described in Example 10 to encode multiple protective antigens to be synthesized and delivered by secretion as well as by lysis. Since particulate antigens including virus-like particles (VLPs) are highly immunogenic, various strategies can be used to synthesize VLPs composed of viral antigens and further engineered to display protective antigens from other pathogens. Although early work used the hepatitis B virus (HBV) core as an antigen delivery platform, it was later found that the Woodchuck hepatitis virus (WHV) core was more manipulatable to display protective antigens as used by us to display the influenza virus M2e antigen (19). In developing a diversity of vaccines to induce protective immunity to the SARS-CoV-2 virus (and to be used to develop a universal vaccine against avian, swine and human influenza viruses), we further improved a regulated delayed lysis vector encoding a codon optimized sequence to encode the WHV core. All sequences to be used in vector constructions were synthesized by Genescript and supplied to us in pET30a vectors. The synthesized sequences were designed and specified by us. To achieve high-level expression in the Salmonella vaccine vectors, we substituted all codons used less often than 5 percent of the time in genes highly expressed in Salmonella with more frequently used codons and made further adjustments so that synthesized sequences had a GC content of about 52 percent, the average in the Salmonella genome. Our first step was to excise the Salmonella codon-optimized WHV core sequence with an inserted influenza M2e sequence from pYA4037 (pUC ori) (19) and replace with a Salmonella codon-optimized sequence encoding the SARS-CoV-2 ACE2 binding domain (BD) S protein sequence (aa 434 to 508) and insert into the AsdA⁺ vector pYA3341 (pUC ori) using NcoI and BamHI restriction sites to yield pG8R334 (FIG. 22). This was accomplished by using the EcoRI and XhoI restriction sites flanking sequences encoding aa 81 (76 in original WHV core) as shown in FIG. 23 providing the nucleotide sequences for the WHV core as originally codon-optimized with the inserted codon-optimized SARS-CoV-2 S gene sequence for aa 434 to 508. The resulting pG8R334 was electroporated into χ12615 with selection for DAP independence on LB agar medium with 0.1% arabinose and 0.1% rhamnose. Cultures were prepared from three independent colonies and synthesis of the WHV core sequence with the ACE2 BD of the S protein measured by western blot analysis using rabbit anti-WHV core antibodies. As expected, synthesis of the chimeric WHV core was dependent on induction with IPTG due to the repression by the LacI synthesized during growth in arabinose-containing medium (to prevent lysis) (FIG. 24). We then further codon-optimized the sequence encoding the WHV core in pG8R316 and pG8R317 using more-preferred codons for Salmonella highly expressed genes and to achieve a GC content closer to the GC content of Salmonella (52%) (FIG. 25). We also changed the codons for the second and third codons to be A-rich to further enhance translation efficiency and added a Dc sequence and a sequence encoding a 6HisTag at the C-terminal end of the WHV core-S (aa434-508) fusion. This codon-optimized sequence encoding the ACE2 BD region of the SARS-CoV-2 spike (S) protein encompassing amino acids 438 to 504 inserted in the WHVcore sequence at amino acid was then inserted into the NcoI-BamHI cleaved pG8R111 (pBR ori) to yield pG8R316 and pYA4594 (pUC ori) to yield pG8R317 (FIG. 25).

### Example 12. Construction of Regulated Lysis Plasmid Vectors Encoding SARS-CoV-2 N Protein Fused to the T3SS Effector SopE to Enhance Induction of T-Cell Immunity

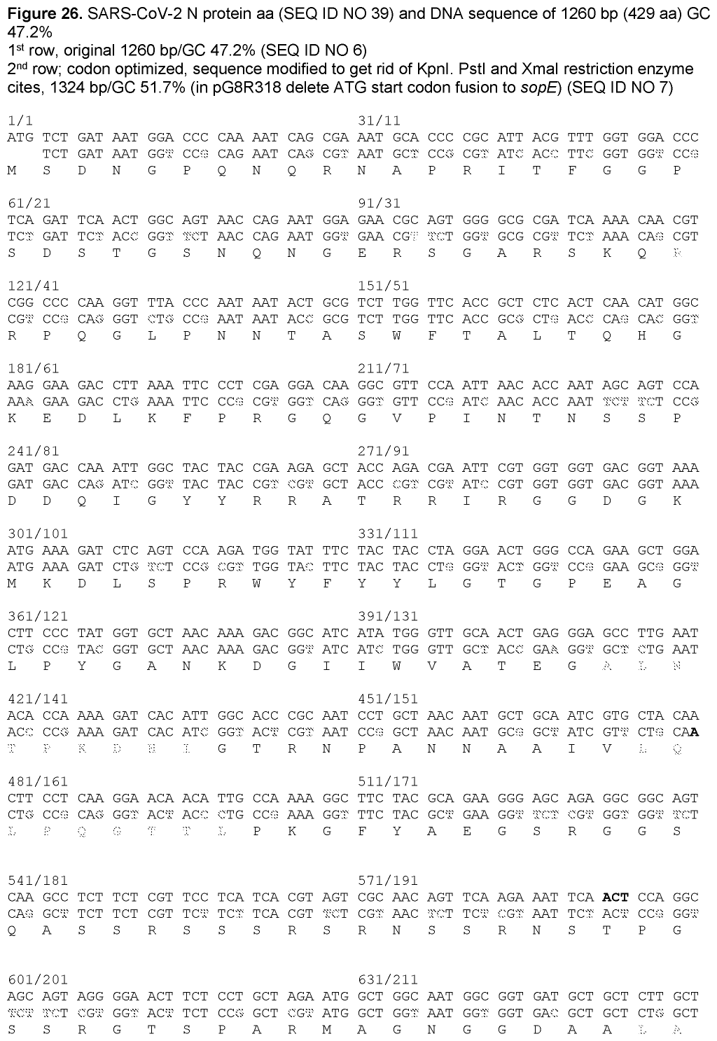

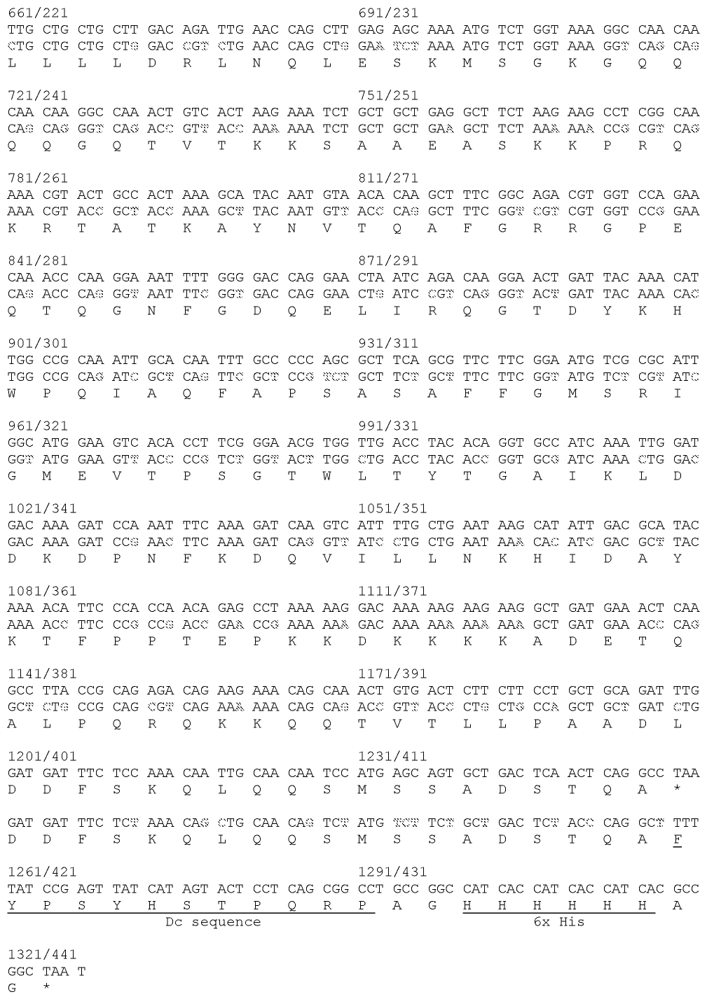

The codon-optimized sequence for high expression the SARS-CoV-2 N gene in Salmonella is presented in FIG. 26. The N gene was cloned into the T3SS vector pG8R110 (p15A ori) cut with XmaI/PstI to generate plasmid pG8R318 (FIG. 25). All plasmids including the control vectors pG8R111 and pG8R110 were transformed into strain χ12615 (Table 3) with selection for DAP independence on LB agar with 0.1% arabinose and 0.1% rhamnose. All constructs were stable for over 50 generations of growth in permissive LB broth media with 0.1% arabinose, 0.1% rhamnose and 50 μg DAP/ml and synthesized S and N antigens after this period of growth. Thus, all plasmids were stably maintained and retain abilities to specify synthesis of encoded SARS-CoV-2 proteins.

### Example 13. Expression of Codon-Optimized Sequences and Synthesis of WHV Core-S Gene Fusions and SopE-N Gene Fusion Specified by Regulated Lysis Plasmids pG8R316, pG8R317 and pG8R318 in χ12615 as Revealed by Western Blot Analyses

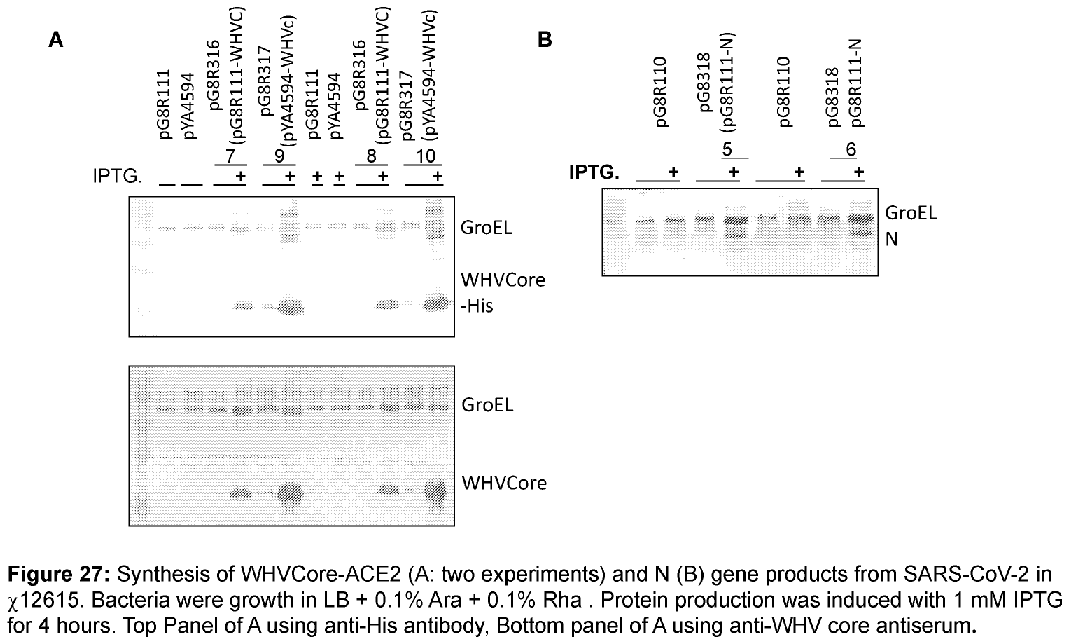

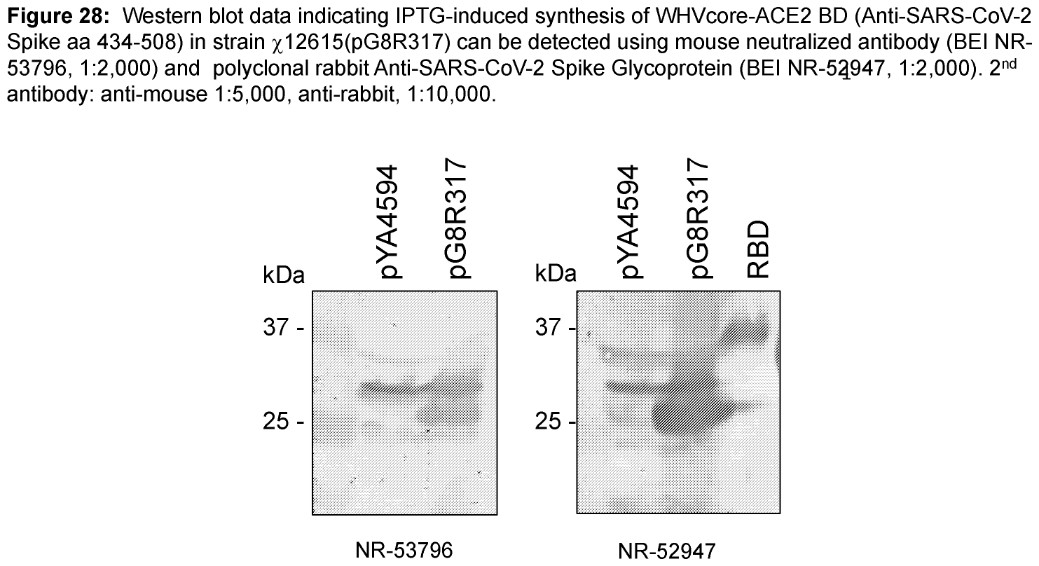

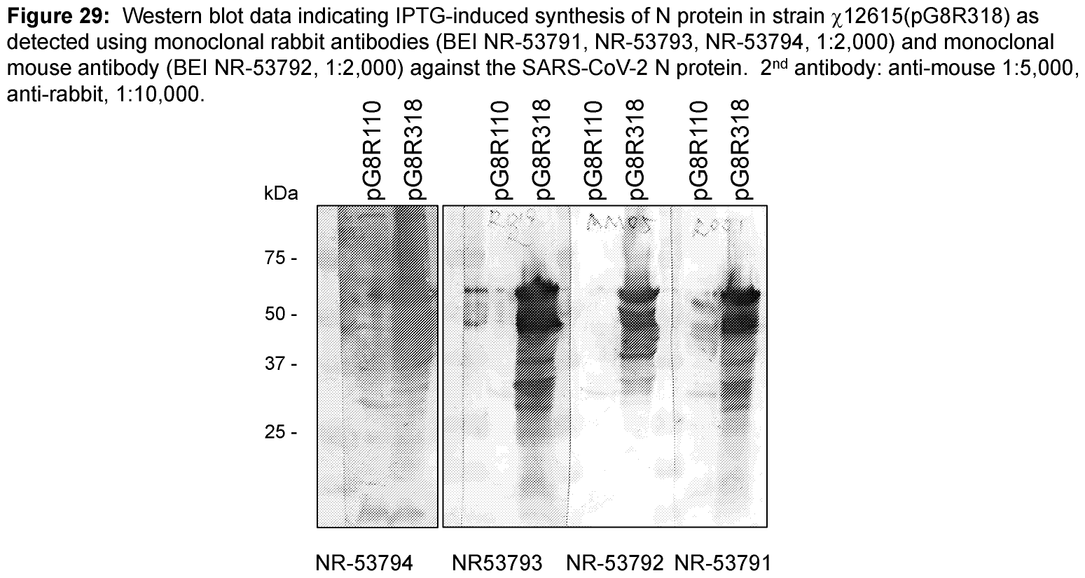

FIG. 27 presents results of these western blot analyses of proteins synthesized without and with IPTG induction to overcome the repression of the P_{trc} promoter by the LacI present due its synthesis in LB broth medium with 0.1% arabinose to preclude strain lysis. Measurement of GroEL was used as a constitutively synthesized protein standard. All constructs behaved as expected with regulated synthesis of the SARS-CoV-2 proteins as fused to the WHV core or to the T3SS effector SopE. FIG. 28 presents analysis of the χ12615(pG8R317) construct for synthesis of the SARS-CoV-2 spike BD domain inserted into the WHV core to be recognized by a mouse monoclonal antibody that can neutralize infection by SARS-CoV-2 as well as by a polyclonal antibody that recognizes the Spike glycoprotein. These immunological reagents were made by others and obtained from BEI. FIG. 29 presents western blot data showing that χ12615(pG8R318) synthesizes and delivers by type 3 secretion the SARS-CoV-2 N protein that is recognized by three rabbit monoclonal antibodies and one mouse monoclonal antibody produced by others and obtained from BEI.

In further improvement in use of PIESV vector systems to generate safe, efficacious and cost-effective vaccines to protect against zoonotic, epidemic and pandemic viral pathogens, the plasmid constructs pG8R316, pG8R317 (Example 11) and pG8R318 (Example 12) can be introduced into the further improved χ12688 and χ12702 PIESV vector strains (Table 3) for use and comparison in more lipid A tolerant and intolerant, respectively, host strains. Studies on stability and regulated antigen synthesis would be repeated in comparison to results presented in FIGS. 27 & 28 as well as conduct of studies in mice as described in Example 15.

### Example 14. Construction of DNA Vaccine Vectors Encoding SARS-CoV-2 S (ACE2BD and Entire Sequence) and N Gene Sequences with and without Codon Modifications

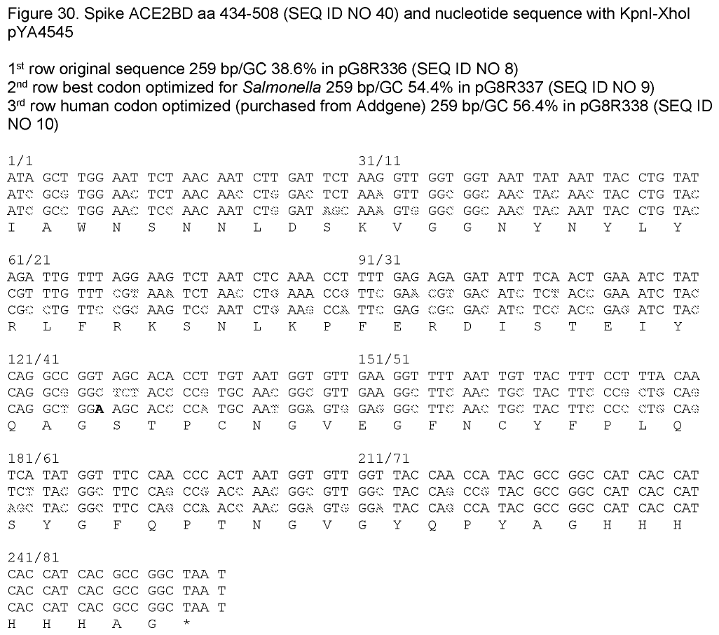

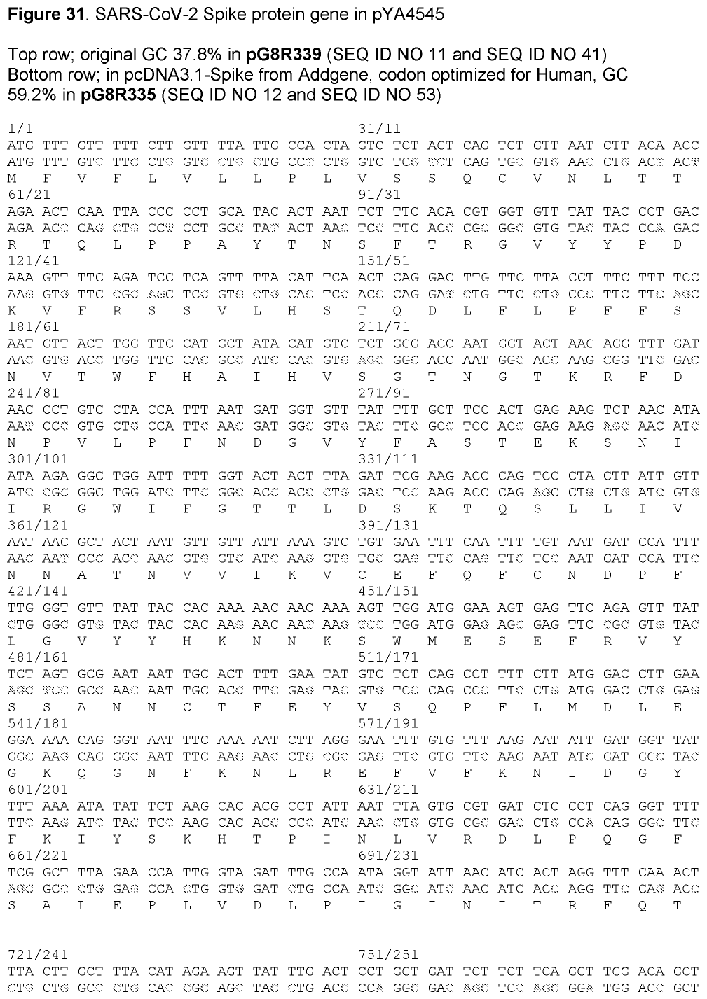

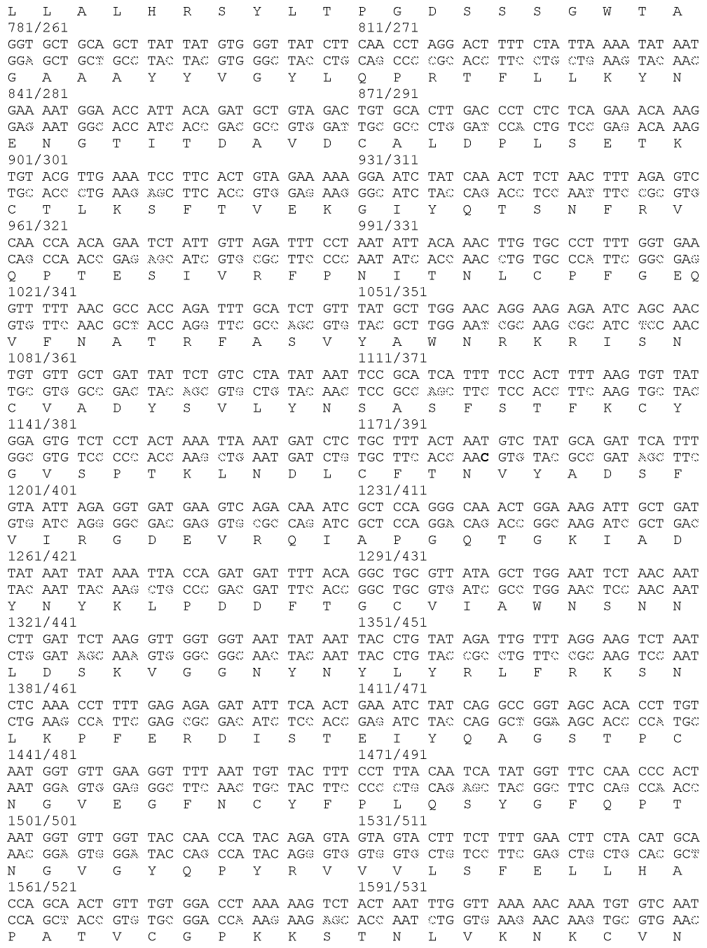

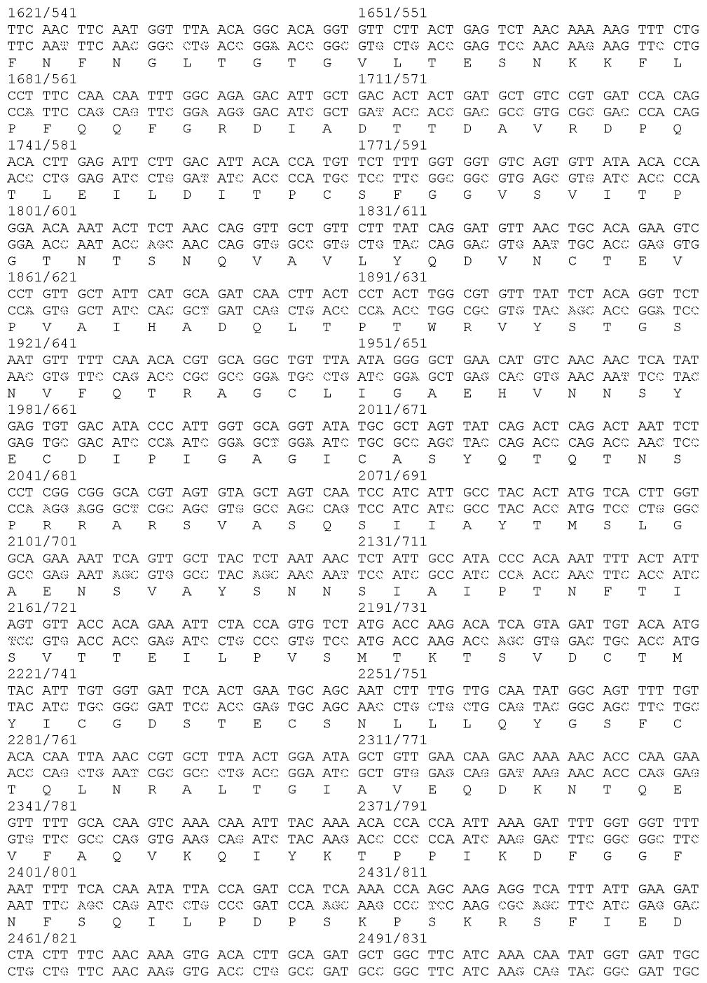

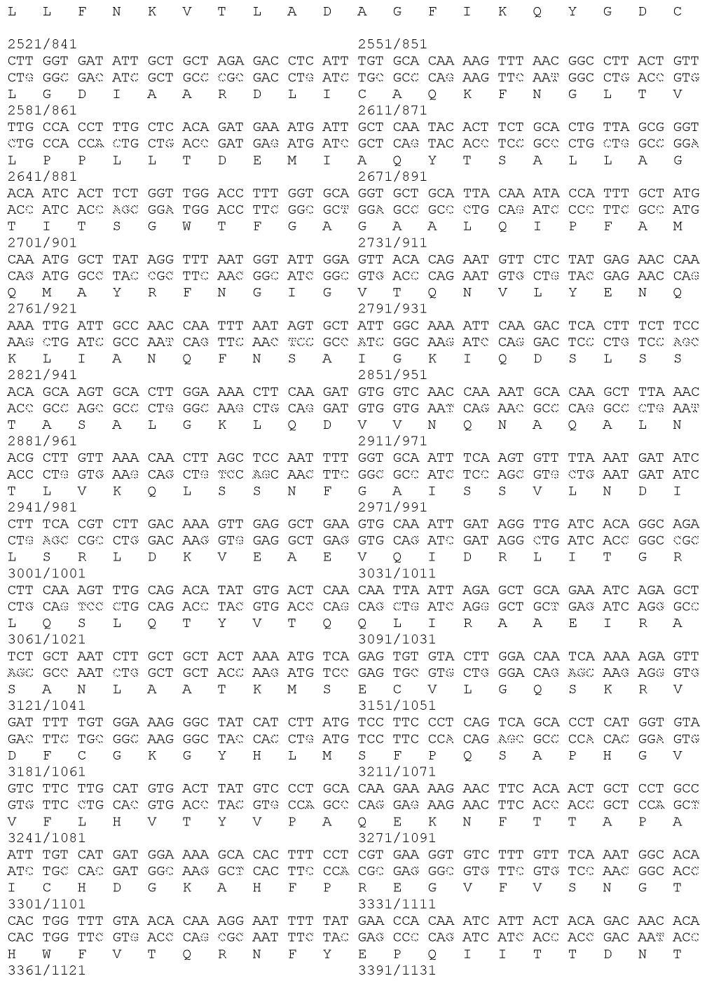

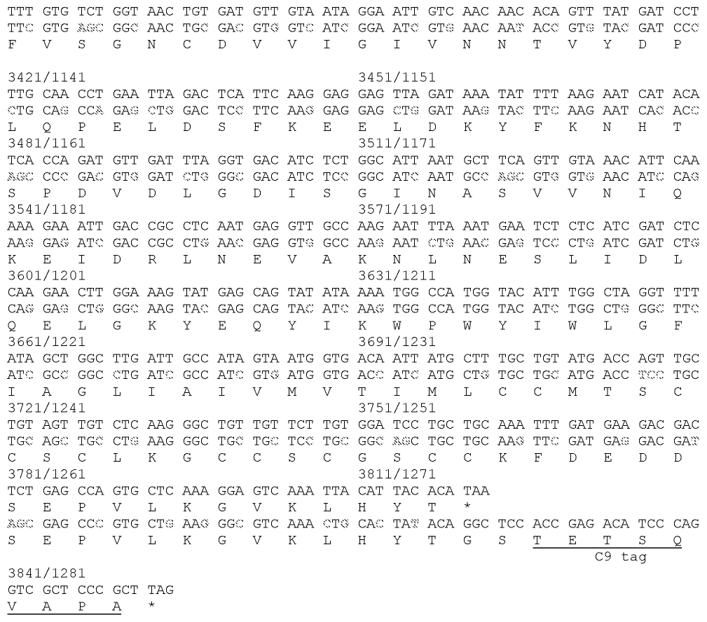

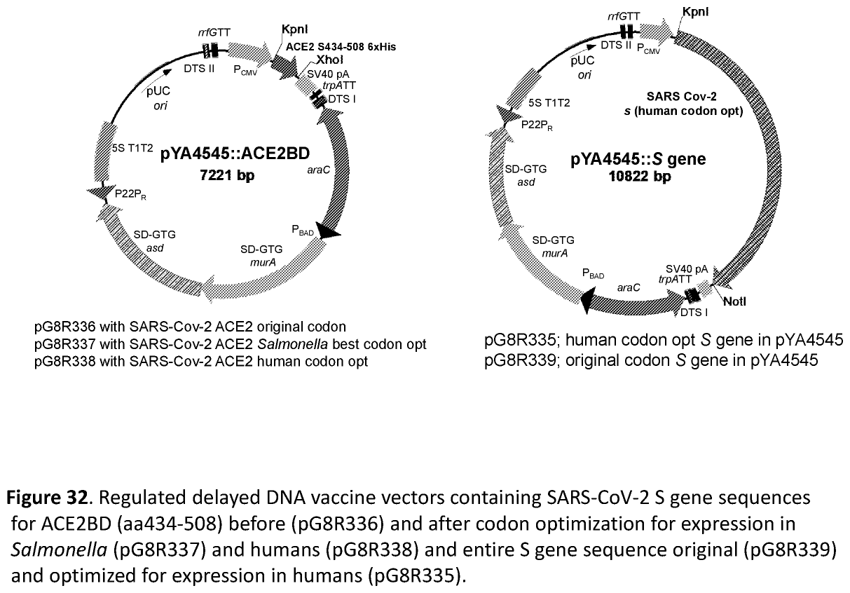

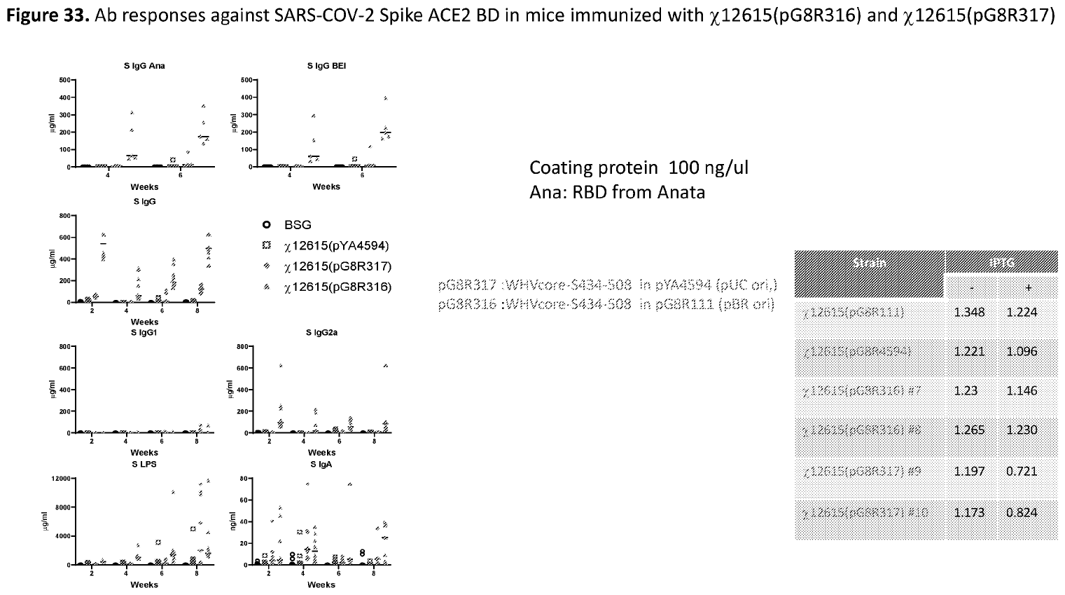

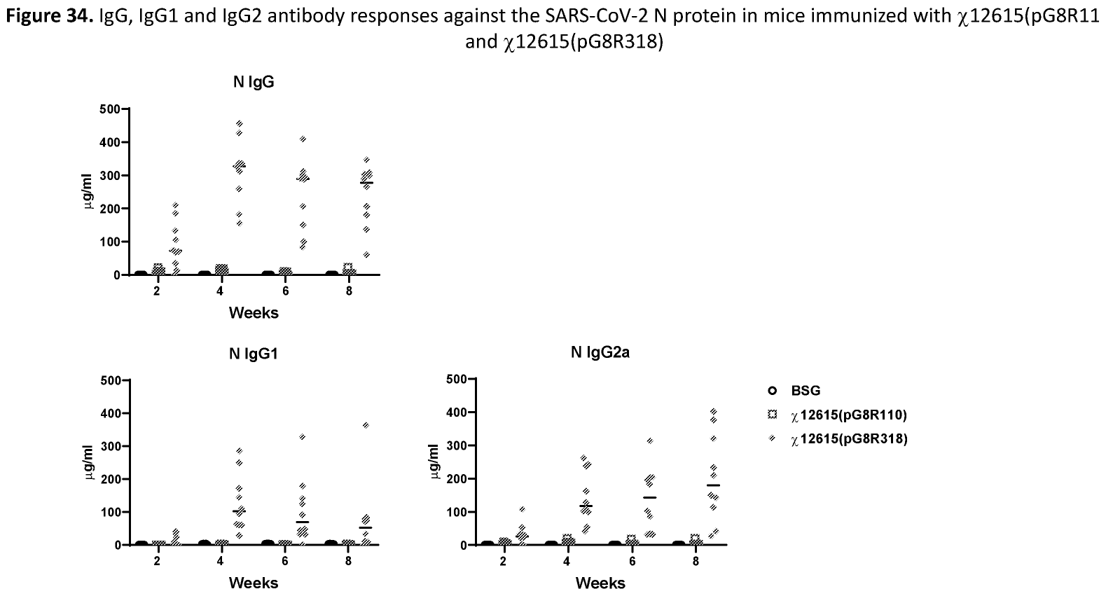

FIG. 30 provides the DNA sequences synthesized by Genescript for the SARS-CoV-2 S gene sequences for spike protein ACE2 BD (aa434-508) to be inserted into DNA vaccine vector pYA4545 with the native sequence (pG8R336), with the codon-optimized sequence for expression in Salmonella (pG8R337) and with the codon-optimized sequence for expression in humans (pG8R338) for delivery by χ12601 (Table 5). FIG. 31 provides the original and human codon-optimized entire S gene sequences for insertion into the DNA vaccine vector pYA4545. The sequences for the original SARS-CoV-2 N gene sequence are given in FIG. 22. FIG. 32 diagrams the structural compositions for the DNA plasmid vectors encoding all these sequences. All of these DNA vaccine vectors were then electroporated into χ12601 by selection for DAP independence on LB agar with 0.1% arabinose and 0.1% rhamnose. All constructs were stable for over 50 generations of growth in permissive LB broth media with 0.1% arabinose, 0.1% rhamnose and 50 μg DAP/ml. Since the χ12601 constructs require multiple cell divisions in vivo prior to commencement of lysis, evaluation of the pYA4545 constructs for ability to specify synthesis of the S and N gene encoded proteins is therefore evaluated after electroporation of the plasmids into Vero cells in culture. In this regard, derivatives of χ12601 with either or both the ΔrhaBADSR515 and ΔaraBAD65::TT mutations have been constructed (χ12642 to χ12645) to determine the effect of timing on time to lyse in vivo impacts the level of DNA vaccine encoded antigen synthesis and therefore induced level of immunity. During making these constructions there was a question as to whether the parameters for timing of attenuation, antigen synthesis, loss of LPS O-antigen and lysis that were optimal for antigen delivery would or would not be optimal for delivery of DNA vaccines encoding protective antigens to maximize induction of protective immunity. The DNA vaccine constructs pG8R335, pG8R336 and pG8R337 in χ12563 and χ12601 (with differing time in rate of loss of O-antigen in vivo), χ12644 and χ12645 (differing in timing of lysis due to inability to catabolize arabinose and rhamnose) and χ12705 and χ12706 (differing in time of lysis due to absence of C2-regulated synthesis of anti-sense RNA for asdA and murA genes) will thus be used to evaluate which genotype of the PIESV DNA vaccine delivery vector will generate optimal results. Alternatively, or in addition, these different PIESV DNA vaccine delivery vectors with the DNA vaccine construct pYA4611 encoding the influenza virus WSN hemagglutinin (95) can similarly be evaluated for highest level of induction of influenza virus neutralizing antibody titer and highest level of protective immunity to challenge of vaccinated mice with influenza virus.

### Example 15. Immune Responses Induced in Mice Immunized with χ12615 with Regulated Delayed Lysis Plasmids pG8R316, pG8R317 and pG8R318

Table 9 lists all the regulated lysis plasmids and DNA vaccine vectors encoding SARS-CoV-2 sequences that have been constructed and characterized for stability and ability to encode synthesis of the S or N protein encoded by the inserted sequences. Each of the lysis plasmids

TABLE 9
List of regulated lysis plasmids and DNA vaccines encoding synthesis or delivery
of SARS-CoV-2 Spike (S) (all or aa434-508 specifying ACE2BD) and N protein

| pG8R | | | | C-terminal |
|---|---|---|---|---|
| number | Gene | Vector | ori | tag |
| pG8R316 | WHVc-ACE2BD S434-508 | pG8R111 | pBR | Dc-6xHis |
| | (Sal best codon) | | | |
| pG8R317 | WHVc-ACE2BD S434-508 | pYA4594 | pUC | Dc-6xHis |
| | (Sal best codon) | | | |
| pG8R318 | N (Sal best codon) | pG8R110 | p15A | Dc-6xHis |
| pG8R334 | S434-508 ACE2BD | pYA4037ΔM2e | pUC | |
| | (Sal best codon) | pYA3341 deriv. | | |
| pG8R335 | ORF S (human codon | pYA4545 | pUC | |
| | opt from Addgene) | | | |
| pG8R336 | S434-508 ACE2BD (orig codon) | pYA4545 | pUC | 6xHis |
| pG8R337 | S434-508 ACE2BD | pYA4545 | pUC | 6xHis |
| | (Sal best codon) | | | |
| pG8R338 | S434-508 ACE2BD | pYA4545 | pUC | 6xHis |
| | (human codon opt) | | | |
| pG8R339 | ORF S (orig codon) | pYA4545 | pUC | |

have been electroporated into χ12615 and the DNA vaccines into χ12601. After testing for stability during permissive growth for over 50 generations the χ12615(pG8R316), χ12615(pG8R317) and χ12615(pG8R318) constructs were grown in LB broth with 0.1% arabinose and 0.1% rhamnose and prepared for vaccination of BALB/c mice as described in Example 1.

Data from immunizing BALB/c mice with the regulated delayed lysis strains χ12615(pG8R316) and χ12615(pG8R317) are presented in FIG. 33. Both strains have Salmonella codon-optimized sequences encoding the SARS-CoV-2 Spike protein aa sequence 434-508 in a Salmonella codon-optimized WHV core sequence. Mice were immunized at 0 and 2 weeks with 10⁹ CFU of the PIESV constructs and serum and vaginal wash samples were collected at weeks 2, 4, 6 and 8. The empty vector control induced no antibodies against the Spike protein but did induce some antibodies against Salmonella LPS. As revealed by the data on growth of strains with and without IPTG, it is apparent that the pG8R117 construct, which has a pUC ori, produces so much of the WHV core fusion protein so as to inhibit growth as revealed by the OD₆₀₀ values in the chart to the right of the Figure. The IgG responses and especially the vaginal IgA concentrations of antibodies induced are thus lower than desired. We presume and will test, that the χ12615 vector strain is producing too much LacI such that antigen synthesis does not initiate as soon as desired before lysis occurs to preclude an adequate level of antigen synthesis. The means to rectify this likely problem are presented in Examples 8 and 9.

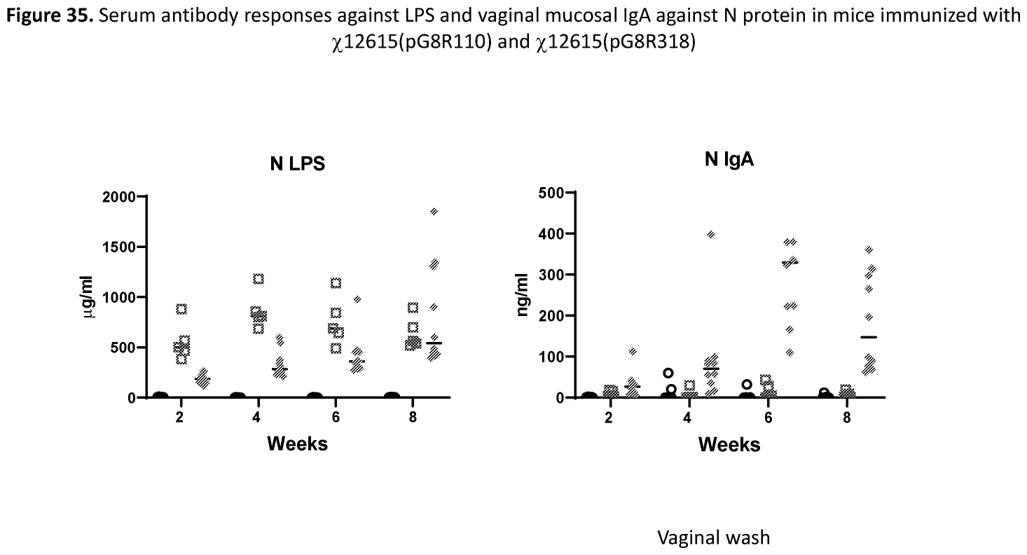

Data from immunizing BALB/c mice with χ12615(pG8R318) that delivers the SARS-CoV-2 N protein antigen encoded by a Salmonella codon-optimized sequence by Type 3 secretion that commences soon after vaccination as well as upon lysis of the PIESV vector are presented in FIG. 34. This construct induced robust immune responses that are mostly of a Th2 type. In contrast, the empty vector control did not induce any antigen-specific antibody responses. However, the empty vector control strain as well as the χ12615 strain delivering N protein induced good serum antibody responses against Salmonella LPS whereas only the χ12615(pG8R318) construct induced good mucosal IgA titers in vaginal secretions against the N protein (FIG. 35).

### Example 16. Construction and Characterization of Regulated Lysis Plasmid Vectors Encoding Streptococcus pneumoniae Protective Antigens PspA and PhtD

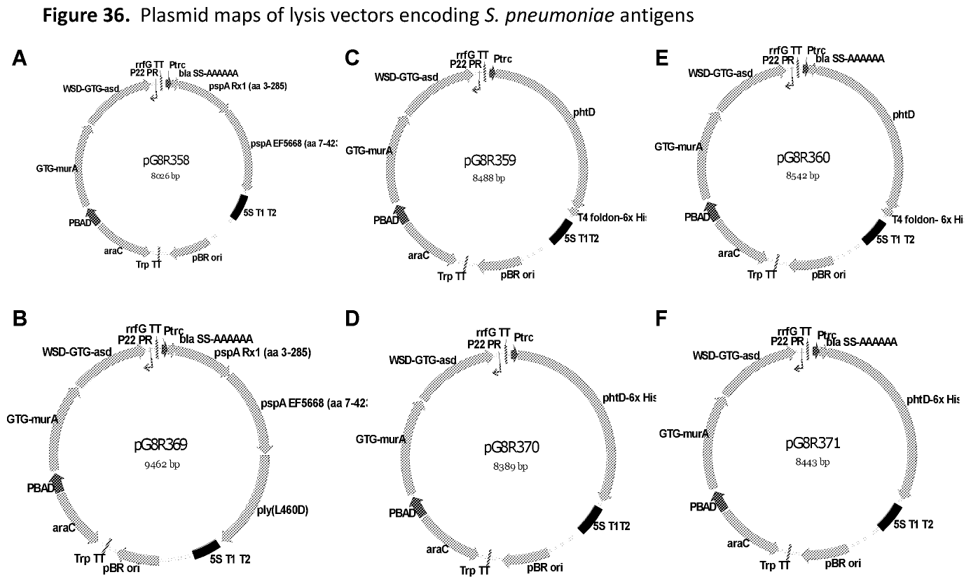

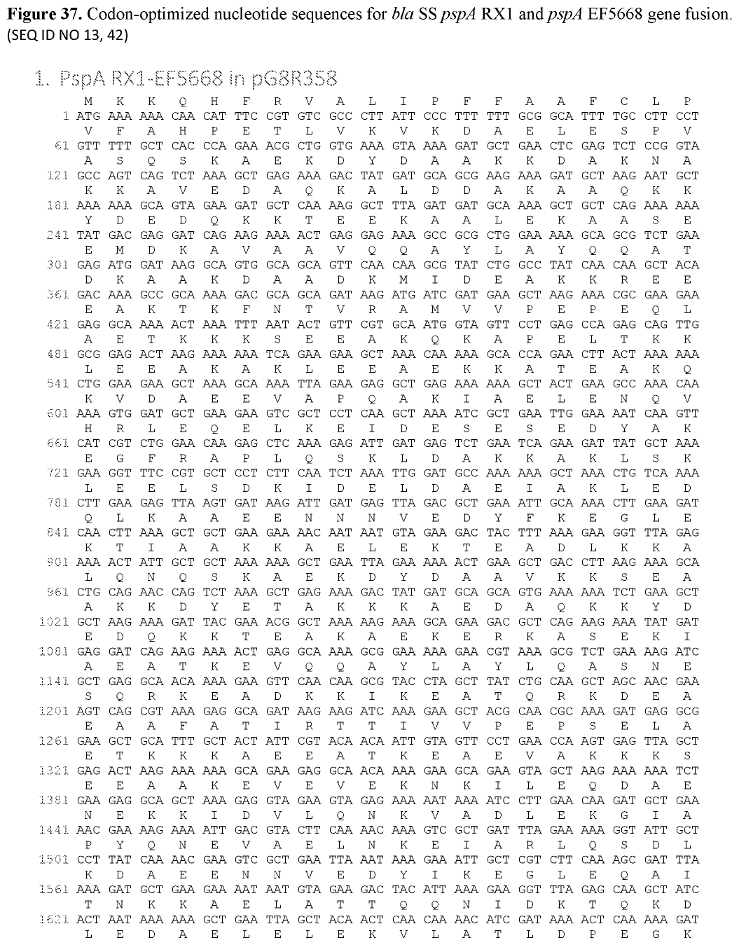

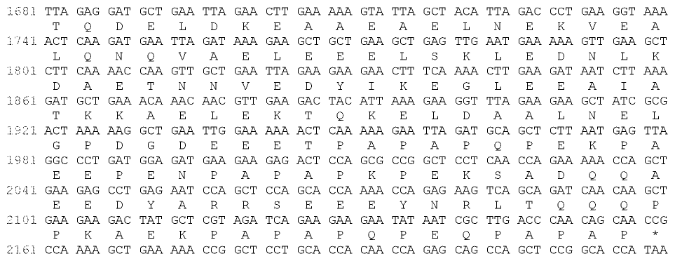

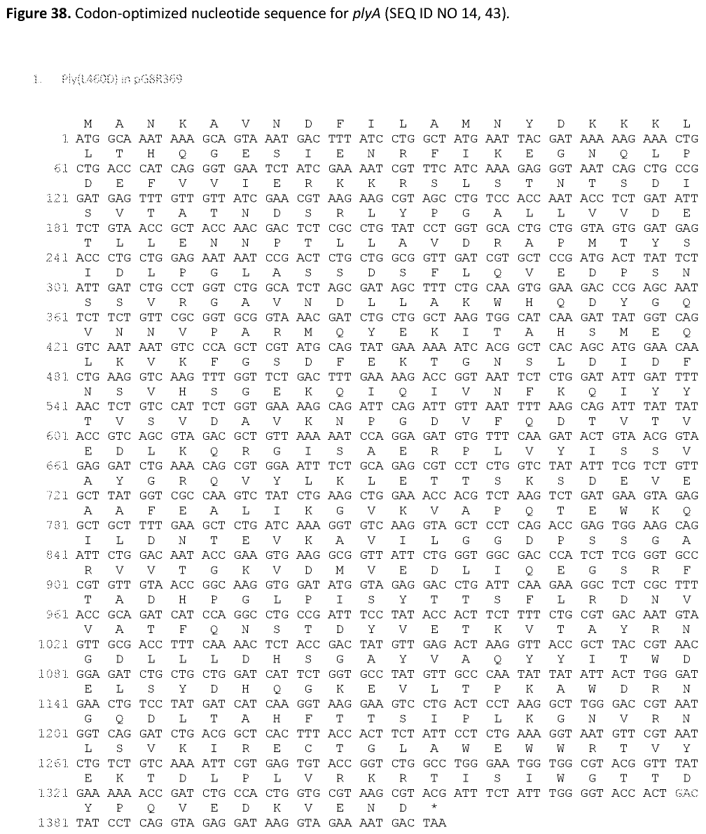

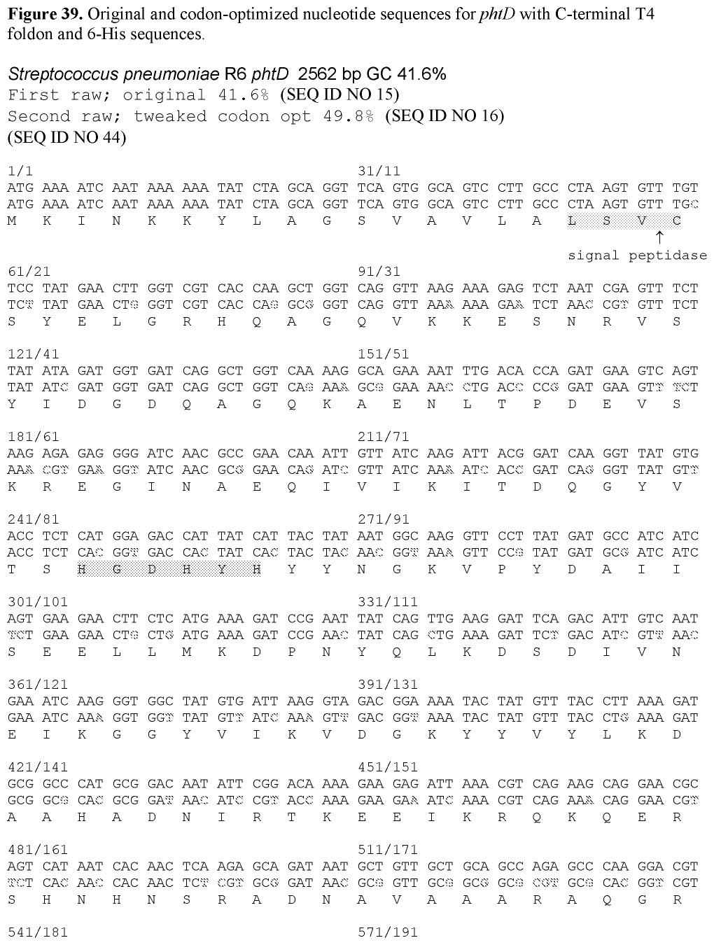

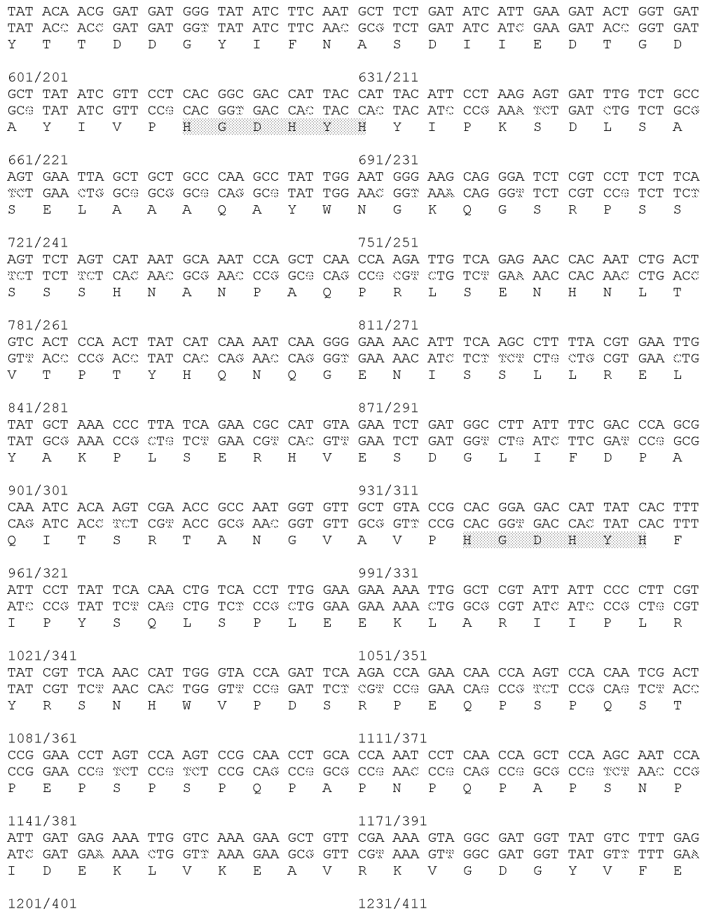

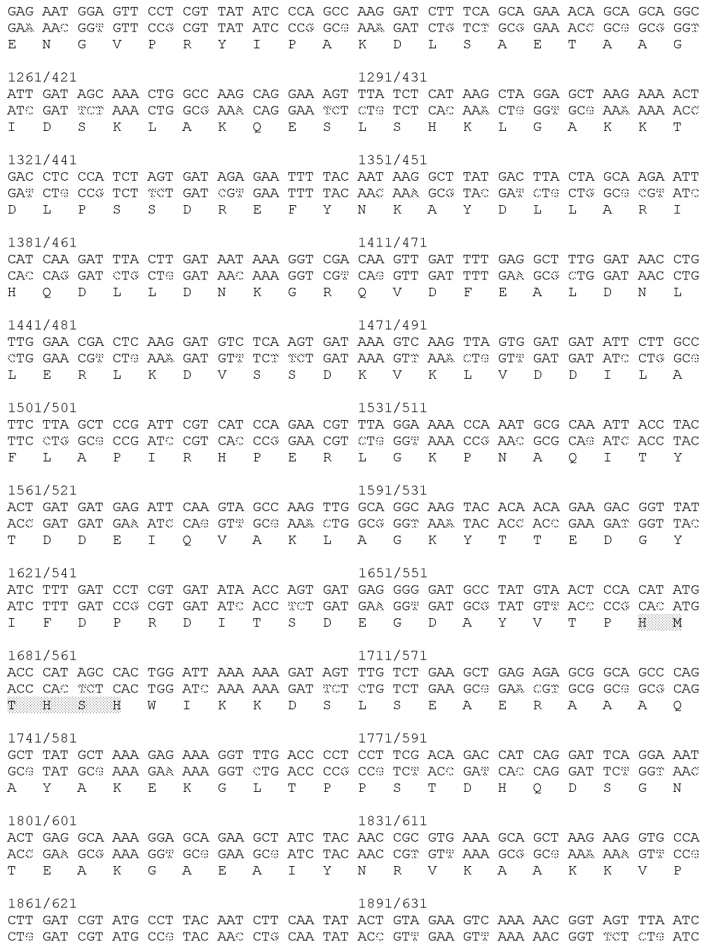

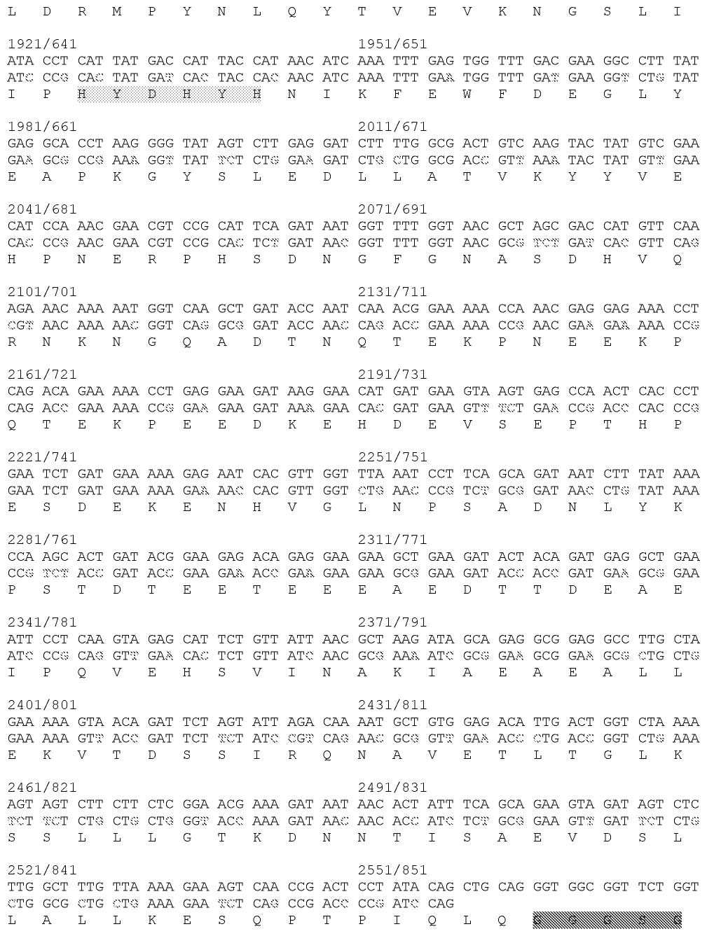

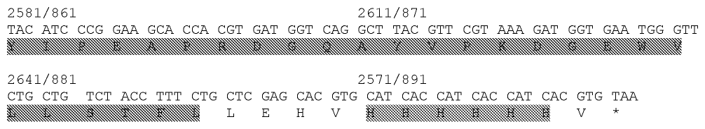

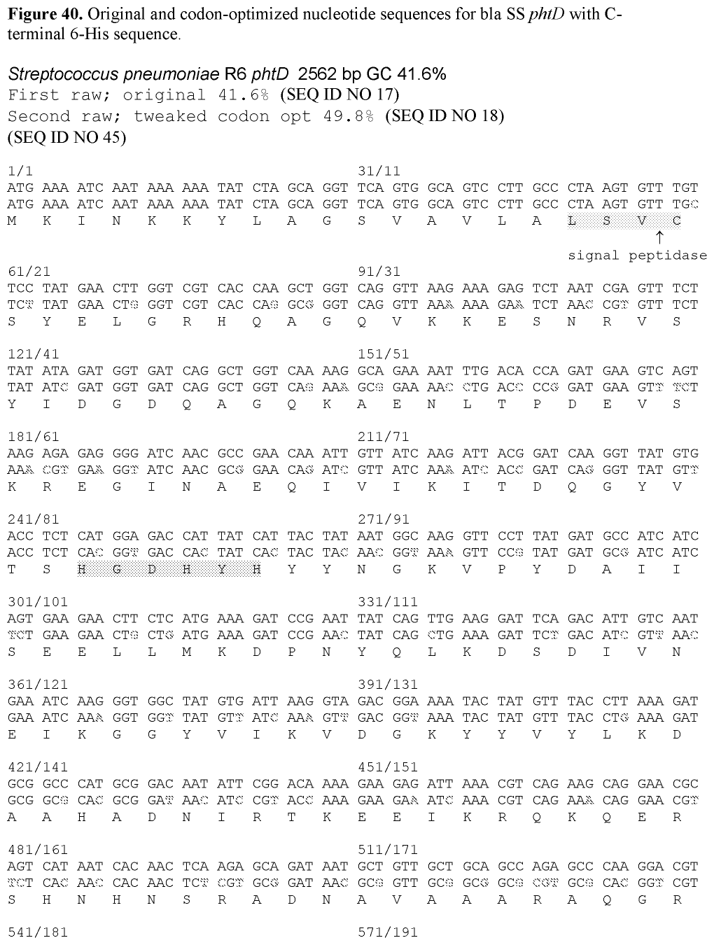

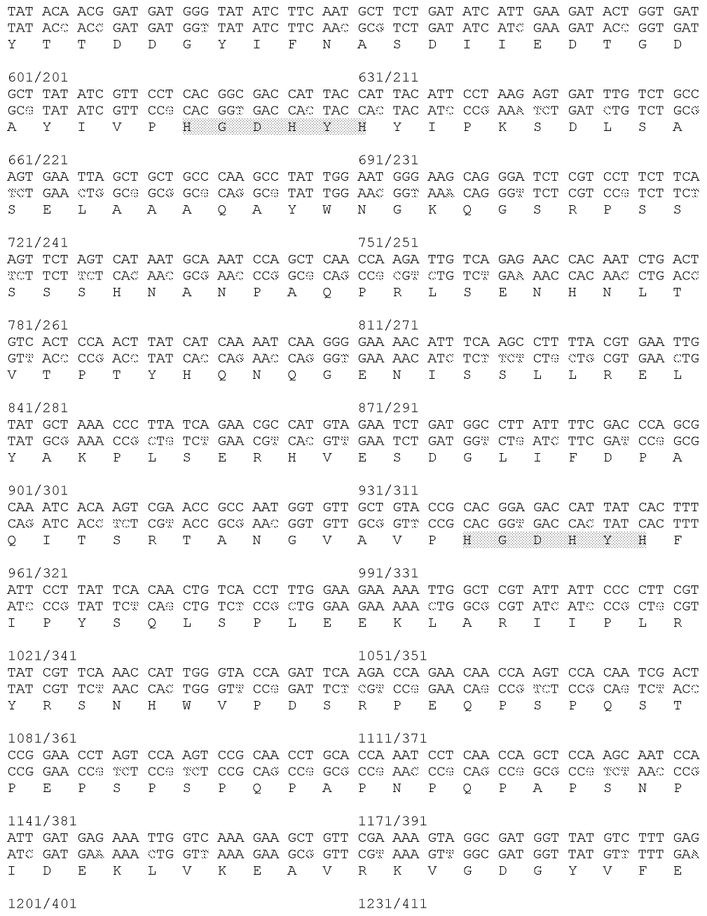

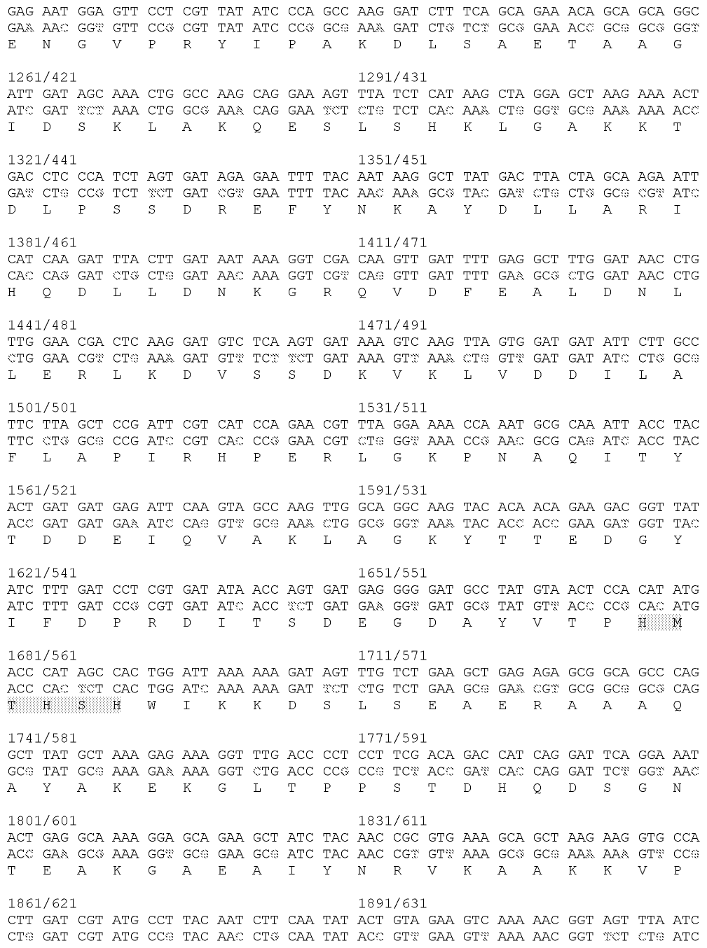

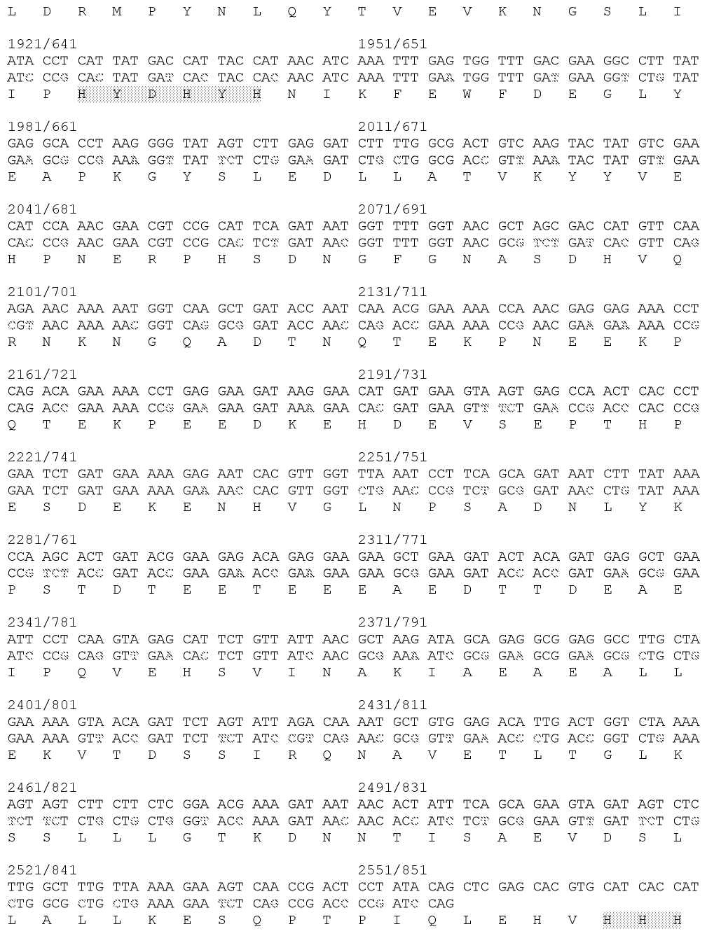

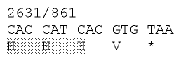

We have been working on the perfection of a PIESV vectored vaccine to protect against all the 95 known serotypes of Streptococcus pneumoniae that cause bacterial pneumonia with very high incidence in children and the elderly. The Pneumovax™ polysaccharide only protects against 23 serotypes, is ineffective in children and not very immunogenic in the elderly. The conjugate Prevnar™ vaccine only protects against 13 serotypes and disease to non-included serotypes is increasing. We therefore have been continually developing increasingly improved safe efficacious vaccines to prevent S. pneumoniae caused disease (11, 16, 24, 168) and have now constructed several improved regulated delayed lysis plasmid vectors encoding an operon fusion encoding a gene fusion of two PspA antigens and PlyA (pG8R369) and the universal PhtD antigen (pG8R370 and pG8R371). The PspA gene fusion encodes epitopes from the two major PspA S. pneumoniae clades (164). FIG. 36 diagrams the constructed plasmids with the codon optimized sequences for the PspA gene fusion listed in FIG. 37, the Ply A sequences in FIG. 38 and the PhtD sequences in FIGS. 39 and 40.

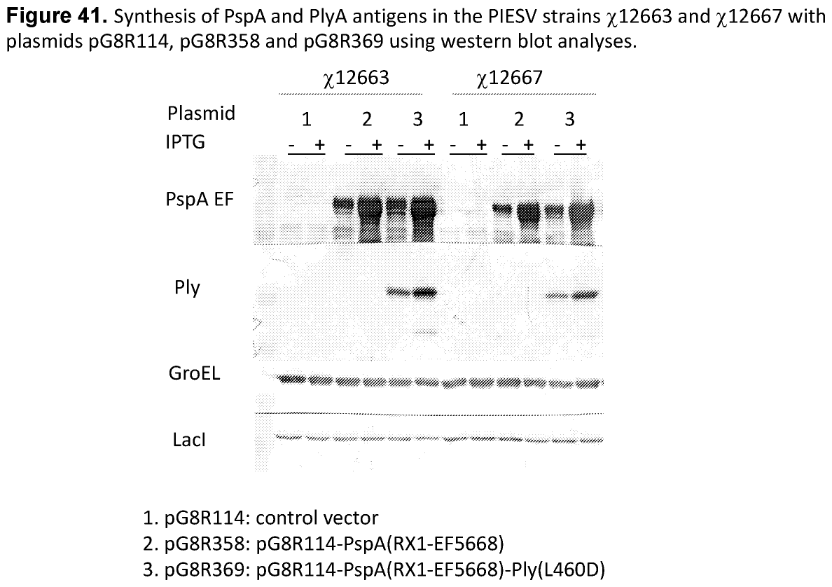

The plasmids pG8R358 and pG8R369 (FIG. 36) were initially electroporated into E. coli χ6212 to be evaluated for ability to synthesize the PspA and PlyA antigens and were then transferred into χ12663 (ΔP_{murA25}::TT araC P_{araBAD} murA ΔasdA27::TT araC P_{araBAD} c2 ΔwaaL46 ΔpagL38::TT rhaRS P_{rhaBAD2} waaL2 Δ(wza-wcaM)-8 ΔrelA1123 ΔrecF126 ΔsifA26 ΔaraBAD65::TT ΔrhaBADSR515 ΔpagP8 ΔlpxR9 (pSTUK206 Δ(traM-traX)-41::araC P_{araBAD} lacI TT)) and χ12667 (ΔP_{murA25}::TT araC P_{araBAD} murA ΔasdA27::TT araC P_{arBAD} c2 ΔwaaL46 ΔpagL38::TT rhaRS P_{rhaBAD2} waaL2 Δ(wza-wcaM)-8 ΔrelA1123 ΔrecF126 ΔsifA26 ΔaraBAD65::TT ΔrhaBADSR515 ΔpagP8 ΔlpxR9 ΔompA11 (pSTUK206 Δ(traM-traX)-41::araC P_{araBAD} lacI TT)) (see Table 3). After determining genetic stability after 50 generations of growth under permissive growth conditions (LB broth with 0.1% arabinose, 0.1% rhamnose and 50 μg DAP/ml) and stability of synthesized PspA and PlyA antigens during 4 hours of protein-synthesis inhibited incubation, these constructs were evaluated by western blot analyses for synthesis of the protective antigens after growth in LB broth (with 0.1% arabinose, 0.1% rhamnose and 50 μg DAP/ml) with and without IPTG induction. The results are presented in FIG. 41. Also, neither PspA or PlyA were synthesized when χ12663 and χ12667 either with no plasmid vector or the empty pG8R114 vector were analyzed by western blotting. Due to the high-level synthesis of the protective PspA and PlyA antigens in both PIESV strains, it is not possible to see whether the deletion of the ompA gene in χ12667 enhanced the levels of the protective antigens synthesized or enhanced their presentation on the cell-surface or in OMVs. Such determinations will best be made from comparative immunization studies with BALB/c mice measuring quantitative titers of antibodies induced and levels of protective immunity to challenge with virulent S. pneumoniae. Such studies using the methods described in Example 1 are in progress.

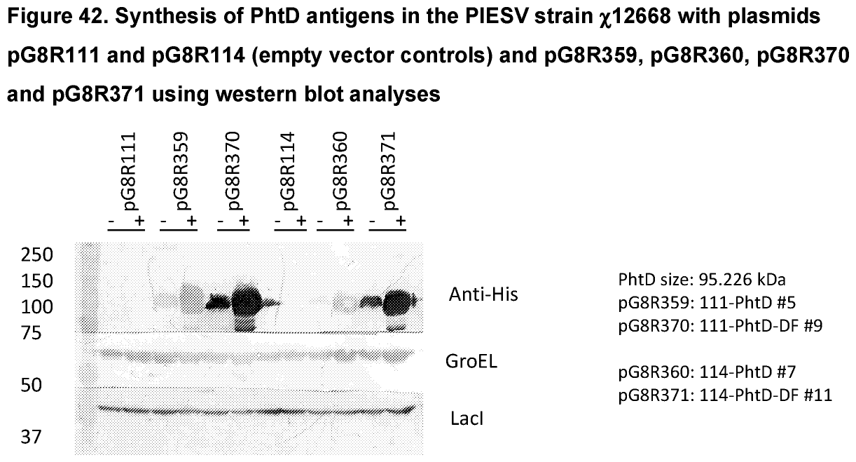

The four plasmids encoding PhtD were made with and without a sequence from the T4 fibritin gene (169, 170) to cause trimerization of the subunit proteins and all with C-terminal His sequences for insertion into pG8R111 (without) and pG8R114 (with the improved bla SS) vectors. After initial introduction and evaluation in E. coli χ6212, they were then transferred into the PIESV strain χ12688 (ΔP_{murA25}::TT araC P_{araBAD} murA ΔasdA33 ΔwaaL46 ΔpagL38::TT rhaRS P_{rhaBAD2} waaL2 Δ(wza-wcaM)-8 ΔrelA1123 ΔrecF126 ΔsifA26 ΔaraBAD65::TT ΔrhaBADSR515 ΔpagP8 ΔlpxR9 (pSTUK206 Δ(traM-traX)-41::araC P_{araBAD} lacI TT)) (Table 3). After testing for genetic purity and stability (Example 1), the χ12688 constructs were evaluated for levels of protein synthesis using western blot analyses. The results are presented in FIG. 42. It is interesting that high-level synthesis of PhtD is observed in χ12688 using vectors with (pG8R371) and without (pG8R370) fusion to the improved T2SS bla SS. On the other hand the inclusion of the C-terminal sequence from the T4 fibritin gene impeded the stable synthesis of the PhtD antigen. These plasmid constructs (as well as revised versions with the C-terminal T4 fibritin gene) in the improved PIESV strain χ12688 are being evaluated by oral immunization of BALB/c mice to determine whether the addition of the C-terminal domain of the T4 fibritin protein causes trimerization to enhance either or both levels of induced antibody or protective immunity to challenge with virulent S. pneumoniae.

Since most S. pneumoniae serotype strains are not adapted to cause disease in mice, the antisera from mice immunized with the constructs inducing the highest levels of antibodies against PspA, PlyA and PhtD will be used to evaluate their abilities to react with S. pneumoniae strains of many serotypes as a measure of potential abilities to protect against the majority of S. pneumoniae serotype strains. We will also make extracts of these different serotypes and use the mouse antibodies in western blot analyses to determine the breadth of reactivity to the surface localized protein antigens independent of capsular polysaccharide serotype.

Based on the results of these studies, we will generate a single regulated delayed lysis plasmid vector encoding all three protective antigens and introduce it into the best PIESV vector strain dependent on ongoing studies to evaluate the various additional improvements as fully described in Example 7.

### Example 17. Evaluation of Regulated Lysis Plasmid Constructs Encoding Brucella melitensis Fusion Antigens in Improved PIESV Vector Hosts

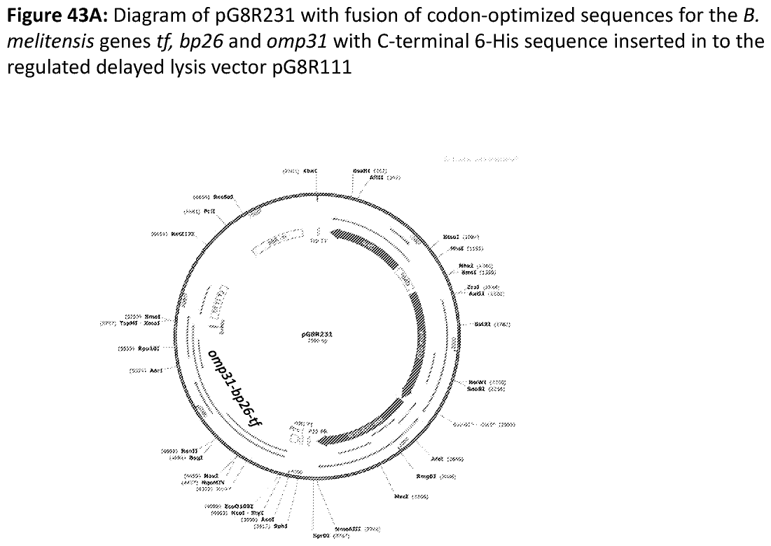

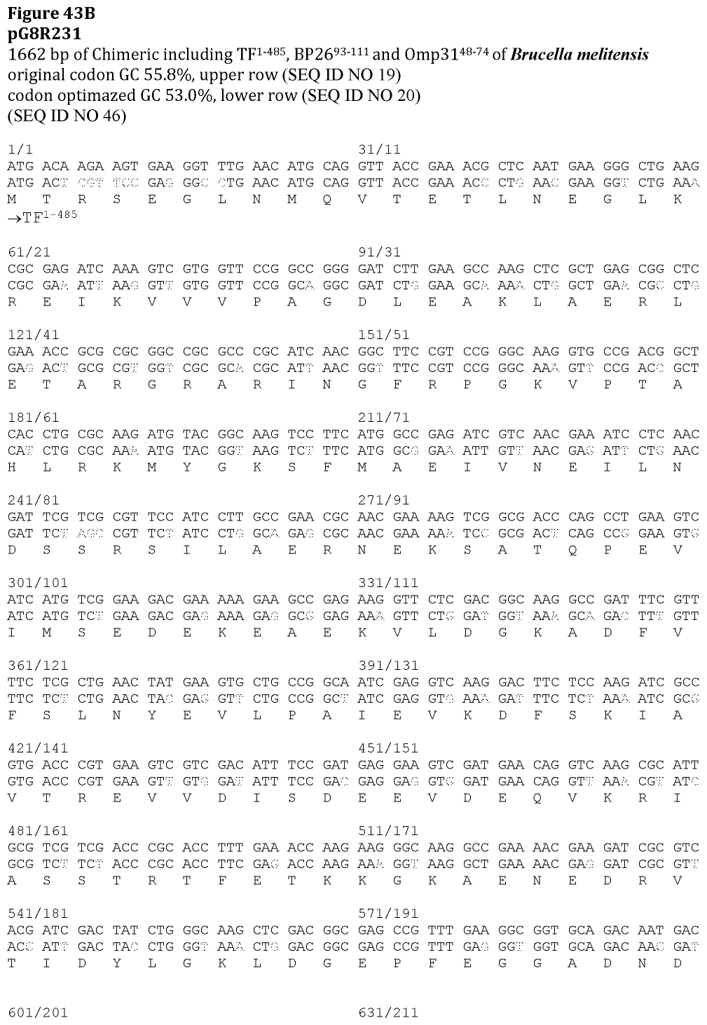

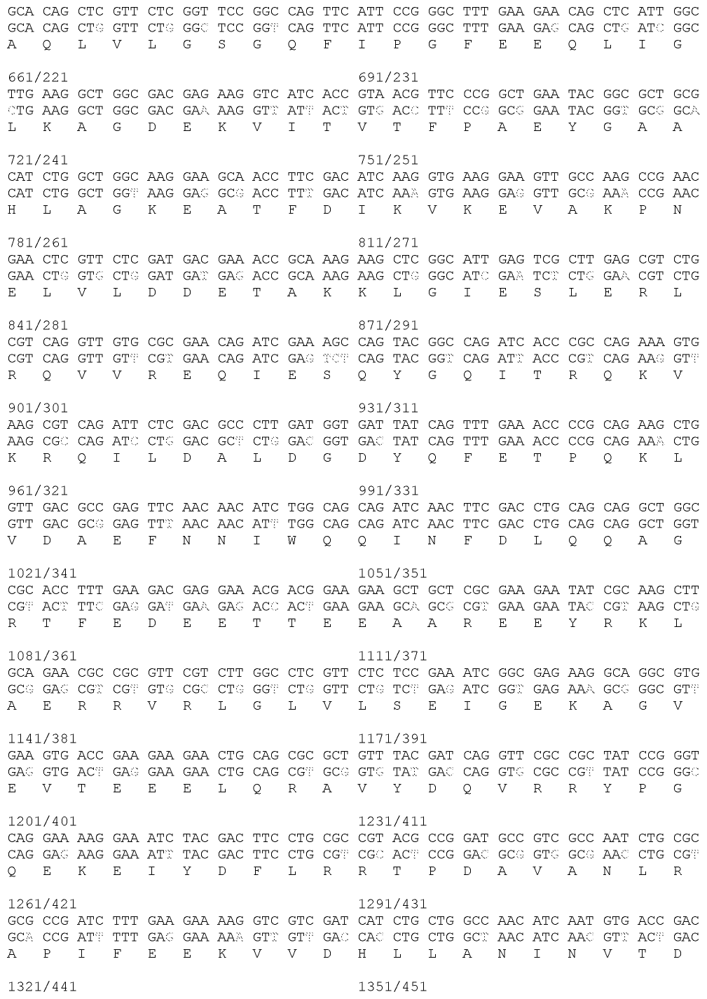

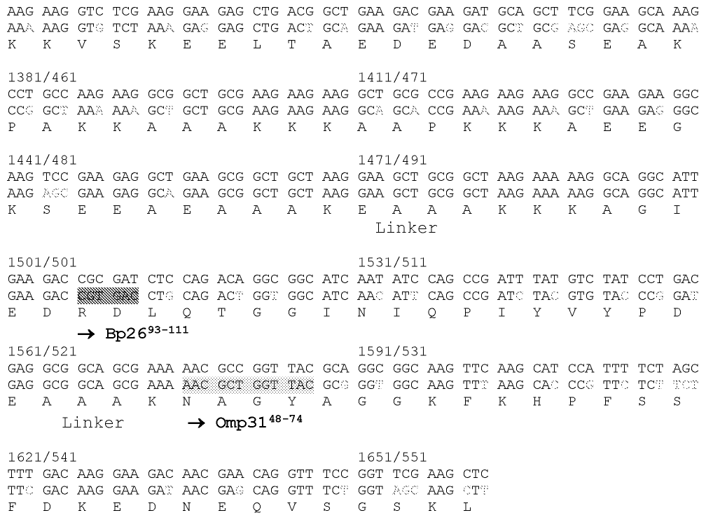

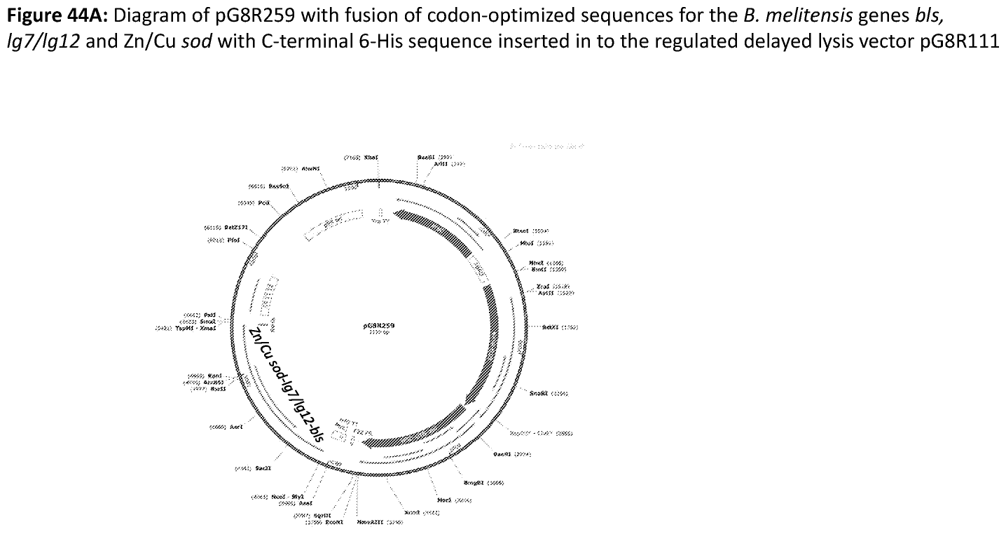

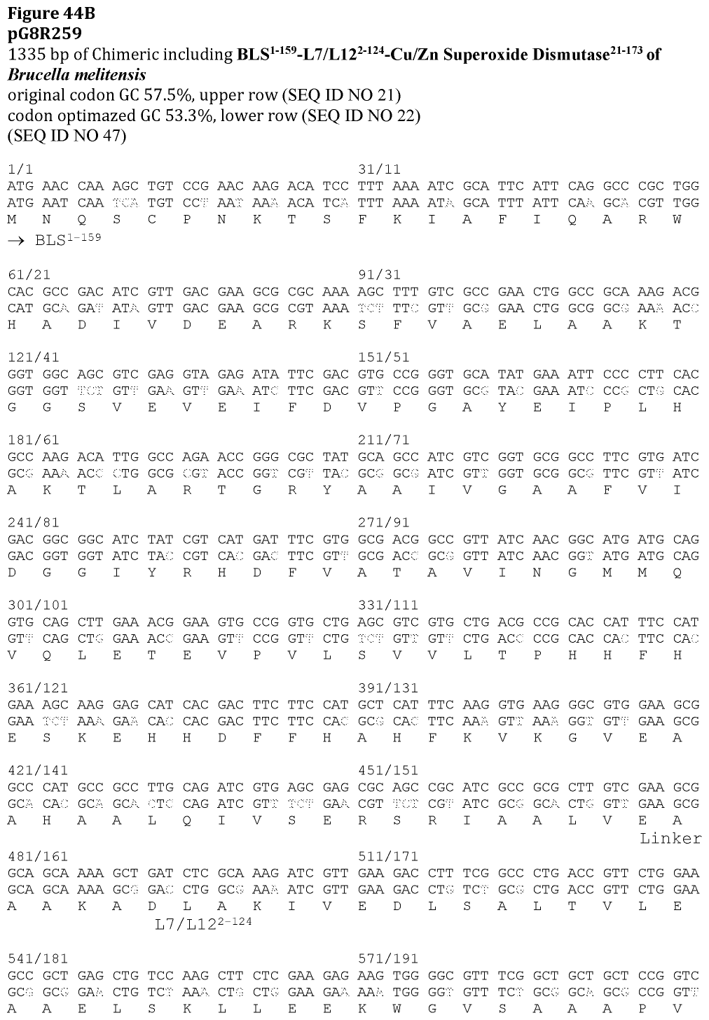

We have developed PIESV vectored vaccines to protect sheep and goats against infection with Brucella melitensis as described in WO 2020/051381 but now plan to evaluate two regulated delayed lysis plasmid constructs each with fusions of three distinct protective B. melitensis antigens. We have thus selected pG8R231 encoding a fusion of the B. melitensis genes tf, bp26 and omp31 (FIG. 43A with nucleotide sequence given in 43B) and pG8R259 encoding a fusion of the B. melitensis genes bls, lg7/lg12 and Zn/Cu sod (FIG. 44A with nucleotide sequence in 44B). Both fusions have a C-terminal His sequence and were constructed by insertion of the sequences into pG8R111 that lacks all secretion sequences. The genetic and phenotypic properties of these vectors have been evaluated in E. coli and S. Typhimurium vaccine vector strains. Since goats and sheep are much more sensitive to the lipid A endotoxin released by in vivo lysing PIESV vector strains than are mice and chickens, we decided to conduct these evaluations comparing safety (tolerability) and efficacy using two strains only differing in synthesis of the unmodified version of lipid A versus the non-toxic mono-phosphoryl lipid A (MPLA) adjuvant form of lipid A by substituting the ΔpagP8 mutation and replacing it with the ΔpagP81::Pₗₚₚ lpxE deletion-insertion that causes synthesis of a Francisella tularensis enzyme that removes the 4′ phosphate on lipid A (68). The isogenic strains that will be used are: χ12688 ΔP_{murA25}::TT araC P_{araBAD} murA ΔasdA33 ΔwaaL46 ΔpagL38::TT rhaRS P_{rhaBAD2} waaL2 Δ(wza-wcaM)-8 ΔrelA1123 ΔrecF126 ΔsifA26 ΔaraBAD65::TT ΔrhaBADSR515 ΔpagP8 ΔlpxR9 (pSTUK206 Δ(traM-traX)-41::araC P_{araBAD} lacI TT) and χ12702 ΔP_{murA25}::TT araC P_{araBAD} murA ΔasdA33 ΔwaaL46 ΔpagL38::TT rhaRS P_{rhaBAD2} waaL2 Δ(wza-wcaM)-8 ΔrelA1123 ΔrecF12 ΔsifA26 ΔaraBAD65::TT ΔrhaBADSR515 ΔpagP81::Pₗₚₚ lpxE ΔlpxR9 (pSTUK206 Δ(traM-traX)-41::araC P_{araBAD} lacI TT) (Table 3).

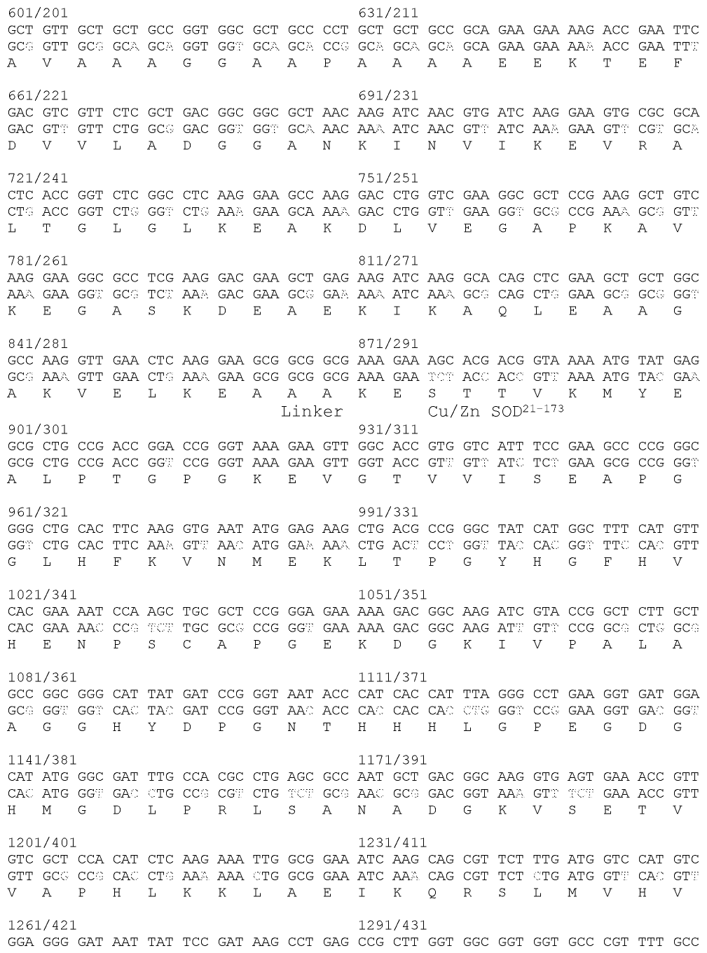

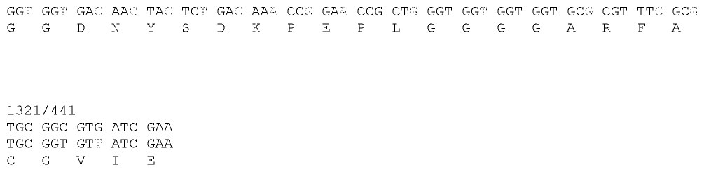

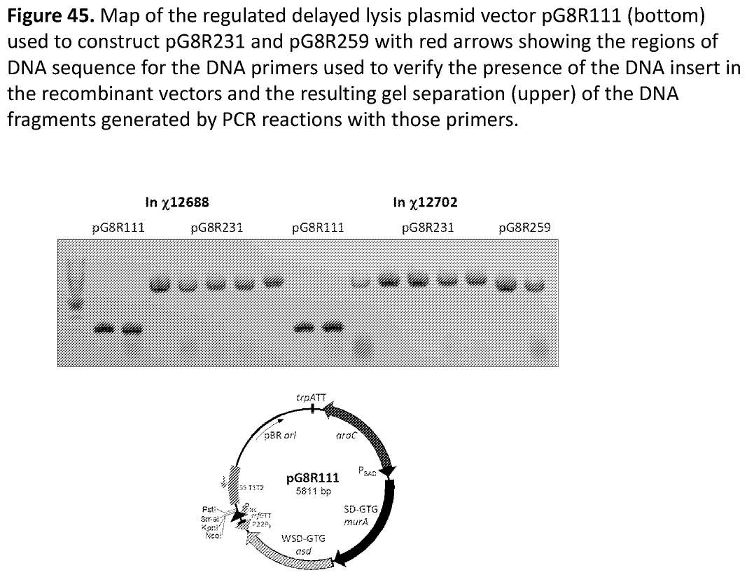

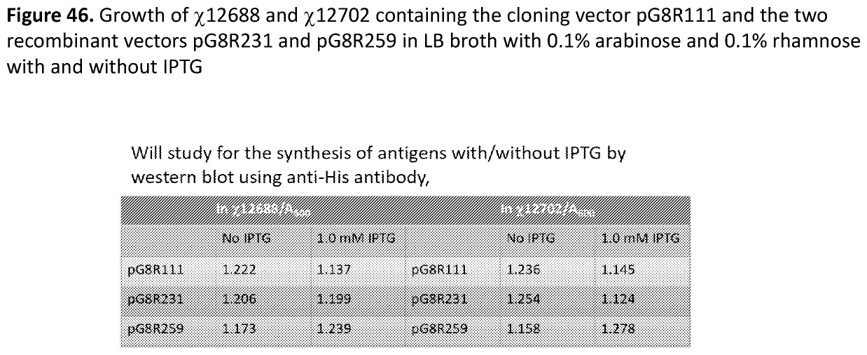

FIGS. 43 and 44 provide diagrams of the maps for pG8R231 and pG8R259, respectively. FIG. 45 provides a map of the regulated delayed lysis plasmid vector pG8R111 used to construct pG8R231 and pG8R259 with red arrows showing the regions of DNA sequence for the DNA primers used to verify the presence of the DNA inserts in the two recombinant plasmids encoding the two different gene fusions after introduction into χ12688 and χ12702 FIG. 46 presents data on the growth of χ12688 and χ12702 containing the cloning vector pG8R111 and the two recombinant vectors pG8R231 and pG8R259 in LB broth with 0.1% arabinose and 0.1% rhamnose with and without IPTG to induce the P_{trc} regulated synthesis of the two fusion proteins. The results indicate that synthesis of the two fusion antigens does not appreciably affect the growth of the two strains. This is a positive result since synthesis of some foreign antigens, especially at high level, can be somewhat toxic to the S. typhimurium vaccine vector strain to reduce invasiveness and desired high-level distribution to internal effector lymphoid tissues in the vaccinated animal host, which can then reduce the level of induced protective immunity.

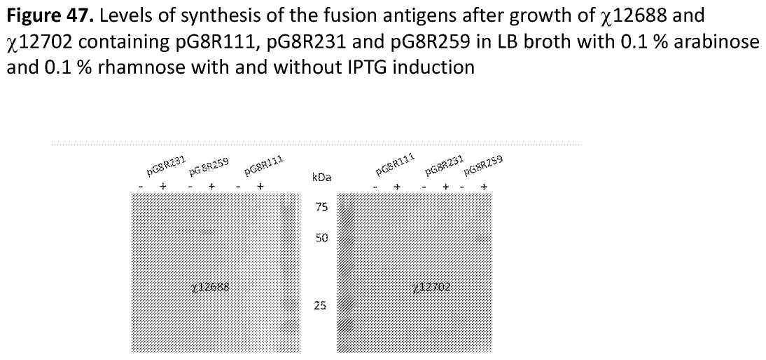

FIG. 47 presents data on the levels of synthesis of the fusion antigens after growth in LB broth with 0.1% arabinose and 0.1% rhamnose with and without IPTG induction by χ12688 and χ12702 containing the cloning vector pG8R111 and the two recombinant vectors pG8R231 and pG8R259. The levels of protein antigens were quantitated by using a monoclonal antibody recognizing the C-terminal His tag. In this regard, detection requires stable synthesis of the entire encoded fusion protein antigens. In this experiment, the levels of antigen synthesis were lower than previously observed (see WO 2020/051381). We are examining this further and are investigating the inclusion of more A rich codons for the 2^{nd} and 3^{rd} codons after the start codon since we previously determined that this enhances translation efficiency to result in higher levels of gene expression to yield protein gene products. We can also modify the SD and spacing sequences to further enhance level of gene expression. If, however, expression is not the problem but rather the stability of the mRNA or the protein, we are adept at determining these other bases for low-level synthesis of protein antigens and rectifying these problems.

The χ12688 and χ12702 strains containing pG8R111, pG8R231 and pG8R259 will be grown under standard conditions, resuspended in BSG after sedimentation at room temperature (see Example 1) and comparatively evaluated by intranasal (i.n.) and intraocular (i.o.) inoculation of young goats. The initial studies with be limited to determine whether the constructs in χ12688 are well tolerated without excessive inflammation or we need to use the χ12702 strain that produces non-toxic MPLA. These initial studies will also use a dose escalation format starting with doses of about 10⁶ CFU administered in 100 μl for i.n. and 50 μl for i.o. routes of vaccination and will increase in increments of 10-fold to doses of up to 10⁹ CFU. Serum and mucosal antibody tiers will be determined as a function of time after primary vaccination. The results will indicate whether a second booster vaccination is warranted. Ultimately, studies will involve challenge with wild-type virulent B. melitensis and even later studies on vaccination of pregnant goats to determine whether B. melitensis induced abortion is prevented. Following these studies, sheep will be used to expand the analyses.

### Example 18. Use of Improved PIESV Vector Strains to Induce Protective Immunity to Bacterial, Viral and Parasite Pathogens

Poultry are frequently colonized by a diversity of bacterial pathogens that can frequently be passed through the food chain to human consumers to cause diseases that are often not apparent in poultry. Parasitic pathogens in the genus Eimeria cause coccidiosis with damage to different segments of the gastrointestinal tract (depending on the species) that result in economic losses to the poultry industry but also facilitate colonization by and intensify disease symptoms by bacterial enteric pathogens. Poultry are also susceptible to infection with avian influenza strains that can cause severe disease but also have the potential to infect swine leading to the possibility of genome reassortment to generate new influenza virus strains that can cause epidemic or pandemic disease in humans.

We therefore have further improved and perfected a PIESV vector strain χ12704 that will induce good to partial protection to poultry against a diversity of bacterial enteric pathogens to both lessen disease in poultry but importantly reduce the transmission of these enteric pathogens through the food chain to humans. It is also expected that the use of this PIESV vector strain to deliver protective antigens from other pathogens will also further improve feed conversion and growth performance. The χ12704 genotype is: ΔP_{murA25}::TT araC P_{araBAD} murA ΔasdA33 ΔwaaL46 ΔpagL38::TT rhaRS P_{rhaBAD2} waaL2 Δ(wza-wcaM)-8 ΔrelA1123 ΔrecF126 ΔsifA26 ΔmntR28 ΔP_{fur33}::TT araC P_{araBAD} fur ΔaraBAD65::TT ΔrhaBADSR515 (pSTUK206 Δ(traM-traX)-41:: araC P_{araBAD} lacI TT). This PIESV vector strain following inoculation into an animal host will cease to synthesize the Fur repressor protein to result in gradual overproduction and display of all proteins involved in iron acquisition. Many of these proteins termed IROMPs are located in the bacterial outer membrane. Since some Fur-regulated genes are co-regulated by the repressor MntR for regulation of manganese-regulated genes, we deleted the mntR gene. This deletion does not alter the invasiveness of the strain. Concomitantly with the increase in IROMPs, there is a cessation in synthesis of the LPS O-antigen (due to cessation in expression of the waaL gene) that ultimately leaves an exposed LPS core that is identical in structure and composition for all 2400 S. enterica serotypes. This results in induction of antibodies that can react with the LPS core of all Salmonella strains and most importantly enhances immunological surveillance of the bacterial cell surface and all outer membrane antigens by the vaccinated animal host. This, in turn, leads to increased immune responses to all the IROMPs and this is important since these proteins are very similar in all enteric bacterial species such that antibodies to the IROMPs made by vaccination with χ12704 react with IROMPs in the outer membranes of a diversity of enteric bacterial species. Other modifications made in χ12704 govern the times for plasmid vector encoded protective antigen synthesis and lysis. These have been optimized to induce maximal mucosal, systemic and cellular immune responses with memory. As described above, strains with a regulated delayed lysis vector (FIG. 1) and the extensive deletion of the tra genes in the pSTUK virulence plasmid ensure a superior level of biological containment.

### Construction of Improved PIESV Strains to Induce Protective Immunity Against Eimeria Infection and Disease.

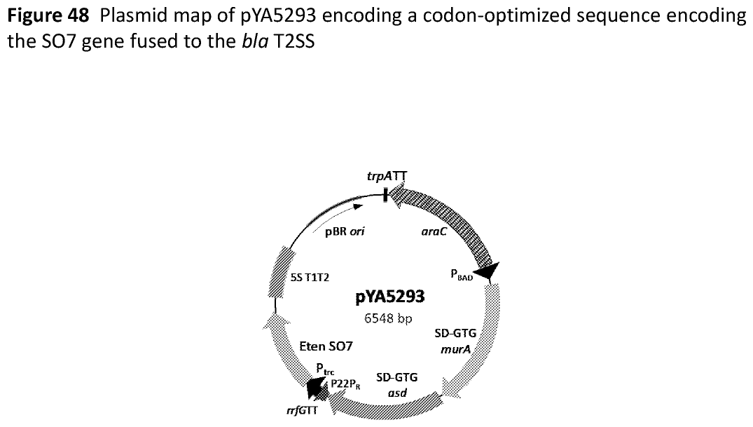

We recently reported (23) induction of protective immunity in chickens vaccinated with a PIESV strain delivering the E. tenella S07 antigen. In this earlier study, the PIESV strain was not designed to induce cross-protective immunity to enteric bacterial species and had few of the improvements present in χ12704. FIG. 48 shows the diagram of pYA5293 that has a codon-optimized sequence encoding the S07 gene fused to the bla T2SS. The bla SS sequence is an older version and is being replaced with the improved sequence generated more recently (22). pYA5293 and several additional regulated delayed lysis plasmid constructs being made to encode additional protective antigens from E. maxima, E. tenella, E. acervulina, E. nacatrix and E. brunetti will be introduced into χ12704. Some antigens such as the E. tenella S07 antigen induce cross-protective immunity to E. acervulina, thus reducing the number of antigens needed to induce a satisfactory level of protective immunity. Constructs after validating genetic and phenotypic stability will be used to spray vaccinate day-of-hatch broiler chicks to be challenged with 10⁴ to 10⁵ Eimeria oocysts at three weeks of age. Feed consumption, weight changes and intestinal lesion scores at the termination of the study at 5 to 6 weeks will be tabulated with feed conversion efficiency calculated. Based on initial results, the sequences for 4 to 5 the most efficacious antigens will be inserted into single regulated delayed lysis plasmid vectors (see Example 10, FIG. 19).

Construction of Improved PIESV Strain to Induce Protective Immunity Against Clostridium perfringens Induced Necrotic Enteritis.

We recently published our results in developing a Salmonella vectored vaccine delivering the C. perfringens PlcC and NetB antigens and also in identifying two other antigens capable of conferring protection against C. perfringens-induced necrotic enteritis (171). As described in Example 10, we have constructed a single plasmid encoding five protective C. perfringens antigens with four of these secreted using four different T2SSs. The pG8R256 plasmid (FIG. 19) has been introduced into χ12704 and this construct will be evaluated for ability to reduce severe and sub-clinical necrotic enteritis in broiler chickens using methods described by Wang et al. (171). χ12704 with the empty regulated lysis plasmid pG8R114 with the T2SS will serve as one control. As observed (171), we anticipate that the empty vector control will afford more protection that observed with the buffered saline control. This is because our improved PIESV constructs serve as very good adjuvants and enhance induction of innate immunity that confers low level protective immunity and also facilitates induction of acquired immunity. It is anticipated that the χ12704(pG8R256) construct will eliminate mortality and weight loss and ensure high feed conversion efficiency. Since coccidiosis and necrotic enteritis constitute the current two major health problems in the poultry industry and entail purchase and use of over half of all drugs used to treat poultry diseases, we anticipate that the vaccine constructs now being made and tested will be highly beneficial to the poultry industry.

### Construction of Improved PIESV Strains to Induce Protective Immunity Against Avian Influenza Viruses of Multiple HA and NA Compositions

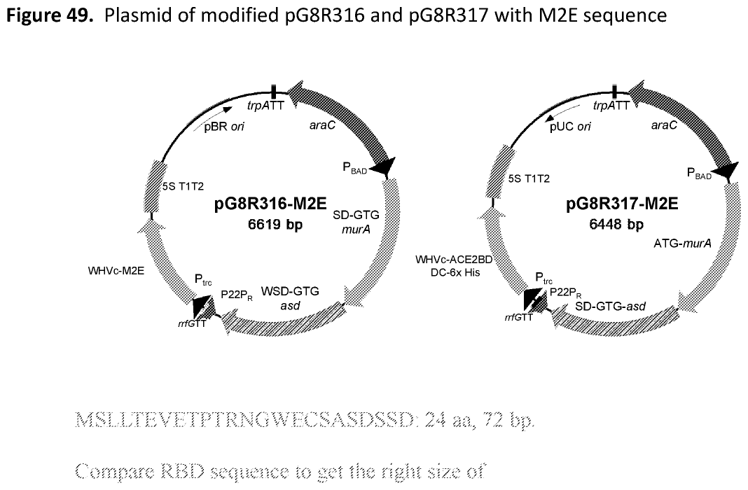

We previously reported on construction of Salmonella vectored vaccines to deliver the highly conserved M2e sequence of influenza virus as a fusion with the woodchuck hepatitis core (19) and used this construct pYA4037 diagrammed in FIG. 22 to generate fusions of SARS-CoV-2 sequences to the woodchuck hepatitis core (Example 11). This involved multiple steps including the further codon optimization of the woodchuck hepatitis core sequence with insertion of Lys codons after the start signal to enhance translation efficiency. The sequence with insertion of the S binding domain (that replaced the sequence encoding the conserved influenza M2e peptide) was inserted into two regulated delayed lysis vectors to result in pG8R316 (pBR ori) and pG8R317 (pUC ori) (FIG. 25). We are now modifying pG8R316 and pG8R317 to remove the S encoding sequence and replacing with the codon optimized sequence encoding the M2e peptide MSLLTEVETPTRNGWECSASDSSD. These two constructs, which are improved over those tested previously (19), are diagramed in FIG. 49 and will be introduced into χ12704 for testing genetic and phenotypic stability prior to conduct of animal studies.

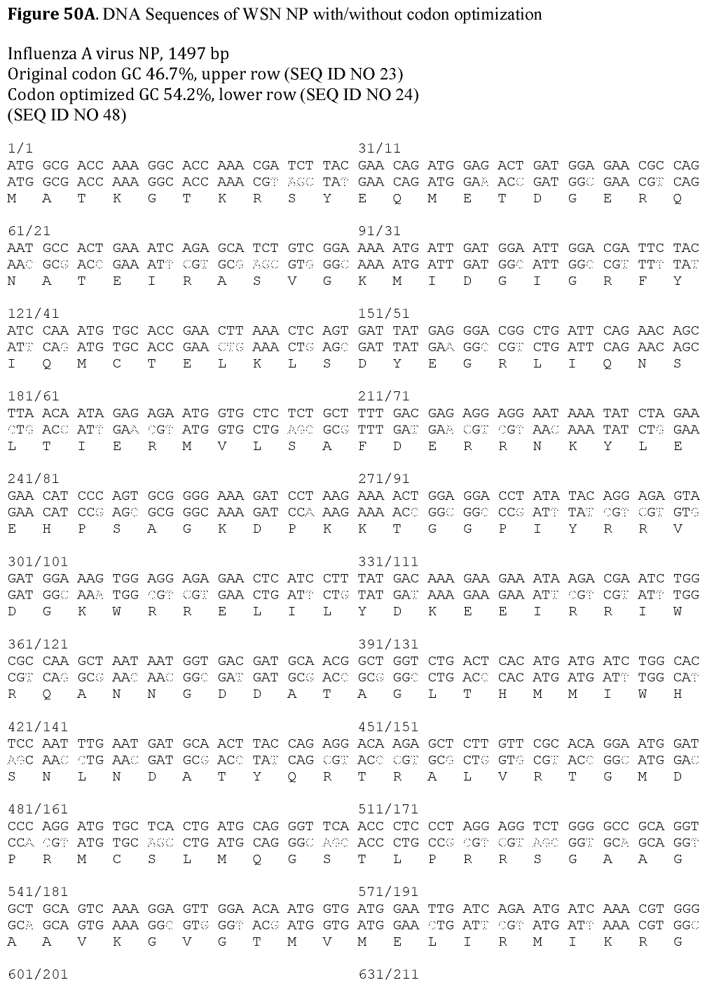

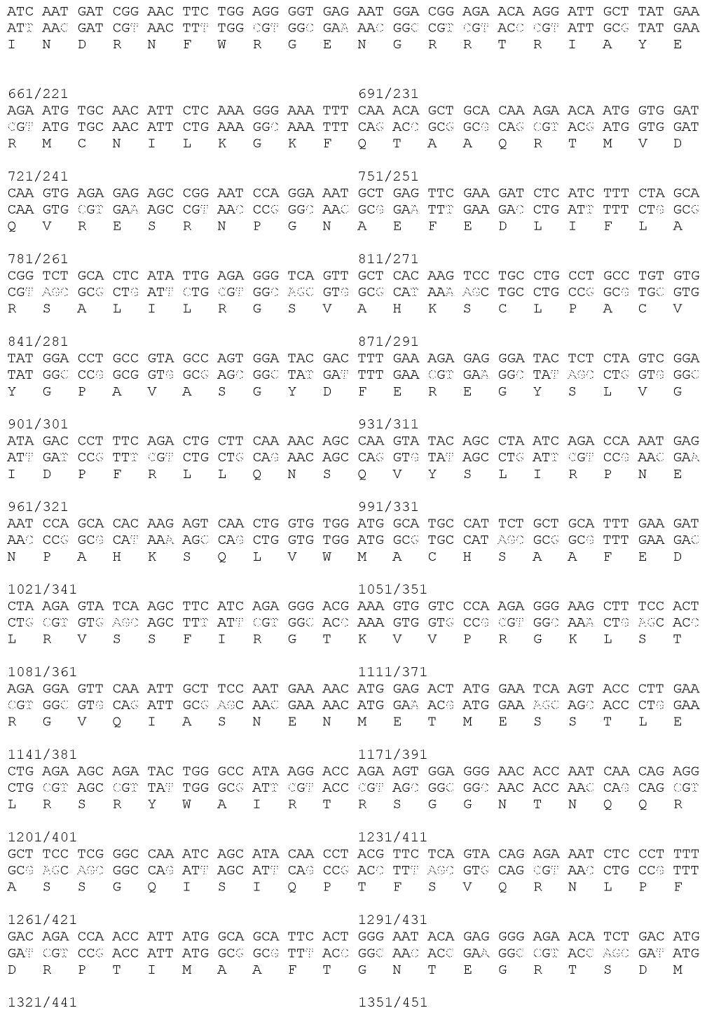

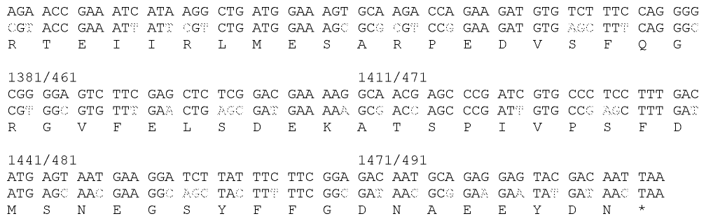

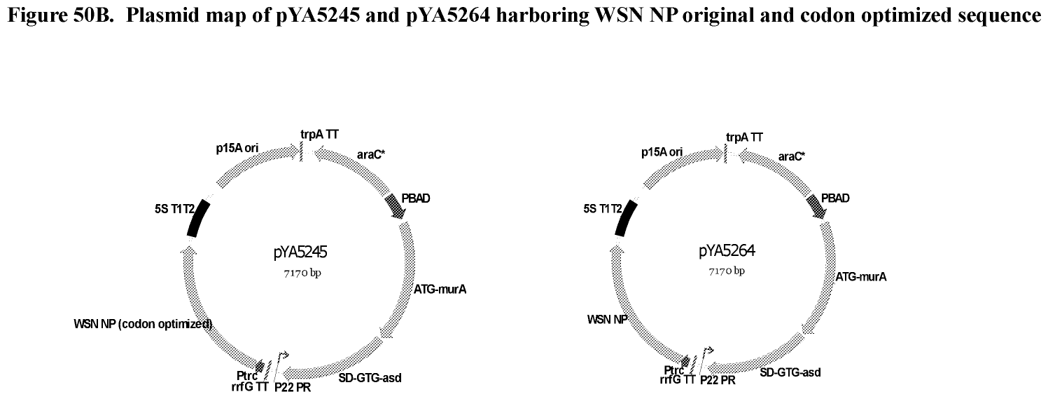

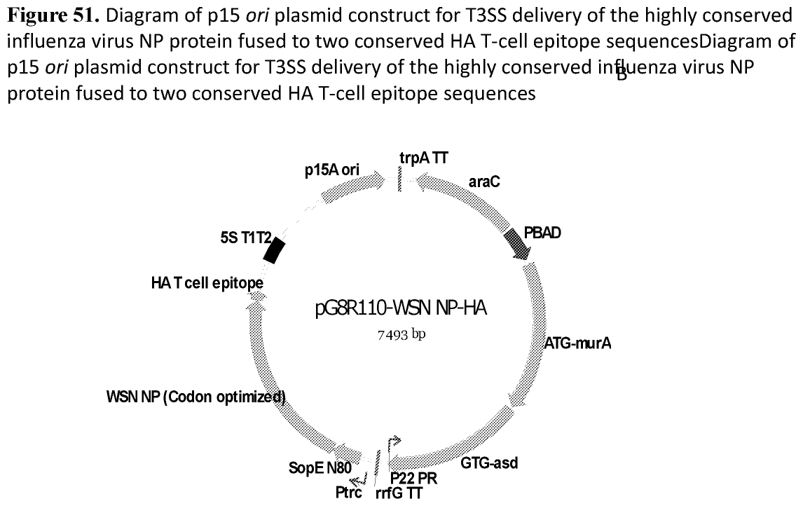

We also previously evaluated induction of protective immunity by immunizing mice with Salmonella vectored constructs delivering the highly conserved influenza NP antigen by a T3SS to enhance induction of cellular immune responses, and especially CD8 responses (20). These were further enhanced by using a regulated delayed lysis Salmonella vaccine vector with a ΔsifA mutation to enable escape from the SCV so that lysis could sometimes occur in the cytosol. Thus, released antigens could be processed by the proteosome for Class I presentation to CD8 cells. Following this initial report (20), we refined the composition to add two conserved T-cell epitopes onto the C-terminal end of the conserved NP protein. This original and codon-optimized sequences given in FIG. 50A are being inserted into the low copy number p15 on pG8R110 vector (FIG. 1) with fusion to the N-terminal first 80 amino acids of the T3 secretion signal SopE to yield the diagramed plasmid vectors (FIG. 50B) and after modification to encode delivery of the conserved NP with the HA fusion to include two conserved T-cell epitopes (FIG. 51).

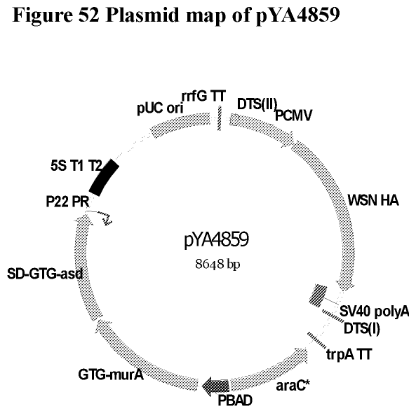

Delivery of viral glycosylated protein antigens by Salmonella leads to poor results since Salmonella is incapable of post translational modification by glycosylation of such antigens. We therefore developed a means by which a Salmonella vaccine vector could deliver a DNA vaccine into the vaccinated animal host to express the encoded antigen genes and process it with appropriate post-translational modifications (95). In that study, we constructed the DNA vaccine vector pYA4545 (FIG. 1) and a derivative pYA4859 encoding the WSN HA gene sequence (FIG. 52). We also determined that the highest level of protective immunity to challenge with the WSN influenza virus was observed when pYA4859 was delivered by the Salmonella vector strain χ11218 (ΔasdA19::TT araC P_{araBAD} c2 TT ΔP_{murA7}::TT araC P_{araBAD} murA ΔaraBAD1923 ΔaraE25 Δ(gmd-fcl)-26 ΔrelA1123 ΔendA2113 ΔsifA26 ΔsseL116 ΔtlpA181 ΔP_{hilA}::P_{trcΔlacO888} hilA). As described in Example 6, we have made substantial changes in the genotype leading to the currently most improved DNA vaccine vector strains (Table 5). We are therefore introducing pYA4859 into χ12705 (ΔP_{murA25}::TT araC P_{araBAD} murA ΔasdA33 ΔwaaL46 ΔpagL38::TT rhaRS P_{rhaBAD2} waaL2 Δ(wza-wcaM)-8 ΔrelA1123 ΔrecF126 ΔsifA26 ΔendA2113 ΔsseL16 ΔtlpA181 ΔrhaBADSR515 ΔaraBAD65::TT) and χ12706 (ΔP_{murA25}::TT araC P_{araBAD} murA ΔasdA33 ΔwaaL46 ΔpagL64::TT rhaRS P_{rhaBAD1} waaL1 Δ(wza-wcaM)-8 ΔrelA1123 ΔrecF126 ΔsifA26 ΔendA2113 ΔsseL16 ΔtlpA181 ΔrhaBADSR515 ΔaraBAD65::TT) to compare with χ11218(pYA48549) for abilities to induce protective immunity to WSV challenged vaccinated BALB/c mice. The only difference between χ12705 and χ12706 is the rate of regulated shut off in synthesis and loss of the LPS O-antigen that might impact the timing of lysis and efficacy in release of intact DNA vaccine molecules capable of being recruited to the cell nucleus for transcription of the encoded HA antigen.

Upon complete characterization of all PIESV constructs described above and verification of their genetic and phenotypic stabilities (see Example 1), groups of 6- to 8-week old BALB/c mice will be orally immunized and then challenged 6 weeks later i.n. with 2 or 3 different doses of WSN influenza virus. Mice will be weighed daily for up to 30 days post challenge. Upon evaluating data on vaccination with each construct, we will select the best strain for delivery of each of the three different antigens. We will then repeat studies using three different mixtures of two PIESV strains and a fourth group with vaccination with a mixture of all three vaccine compositions. The objective is to develop a vaccine that is protective against diverse influenza HN types and useful for vaccination of poultry and swine. It is clear that these further studies will be complex, time consuming and costly.

### Example 19. Use of Improved PIESV Vector Strains to Induce Protective Immunity to Additional Bacterial, Viral and Parasite Pathogens

We have in the past or are currently developing PIESV vectored vaccines against a diversity of pathogens of poultry, swine, ruminants and humans. We have thus in recent years developed vaccines, in addition to those described above, against ZIKA virus (U.S. Pat. No. 11,136,354), Campylobacter jejuni and S. enterica (U.S. Pat. No. 11,000,583), C. perfringens (U.S. Pat. Nos. 9,040,059; 10,988,729: WO 2019/028396), Brucella species (PCT/US19/49825), and Helicobacter pylori (WO 2021/159075 A1). The three classes of improved PIESV vector strains described in Examples 4, 5 and 6 and whose constructions were detailed in Tables 3, 4 and 5 along with further proposed improvements as detailed in Example 7 can be used with the regulated delayed lysis plasmid vectors described in Example 3 in the further improvement and efficacy of these vaccines using the materials, methods and procedures detailed in Example 1 and in the others Examples following.

### Example 20. Use of Self-Destructing Attenuated Adjuvant Salmonella (SDAAS) Strains to Augment Levels of Acquired Immunity Induced by Use of PIESV Vector Strains

As noted in above Examples, the improved PIESV vector strains when used as controls with empty regulated delayed lysis vectors not encoding protective antigens, induced low-level protective immunity after pathogen challenge that is significantly better than observed using controls inoculated with buffered saline. Based on these observations, we perfected the design and construction of Self-Destructing Attenuated Adjuvant Salmonella (SDAAS) strains to use as live adjuvants that can be inoculated into animals by a diversity of mucosal and parenteral routes including in ovo into 18-day old chicken embryos to enhance induction of innate immunity. These constructions with description of their properties and uses are covered in other applications (WO 2020/096994 A1; WO 2021/222696 A1 and PCT/US21/43675).

Two different families of SDAAS strains have been constructed and validated. Family A strains undergo very rapid lysis after in vivo inoculation since their ability to synthesize the rigid peptidoglycan layer of their cell wall is dependent on the supply of the unique essential peptidoglycan constituents DAP and D-alanine that are unavailable in animal tissues. The best presently validated Family A strain is χ12703 with the genotype: Δalr-3 ΔdadB4 ΔasdA33 ΔfliC180 Δ(hin-fljBA)-219 ΔpagP81::Pₗₚₚ lpxE ΔlpxR9 ΔpagL7 ΔeptA4 ΔarnT6 ΔsifA26 ΔrecA62. Family B SDAAS strains undergo several cell divisions in vivo prior to their extensive lysis since the synthesis of enzymes needed for the synthesis of DAP and D-alanine are dependent on the supply of arabinose and rhamnose. These enzymes cease to be synthesized in vivo due to the absence of non-phosphorylated arabinose and rhamnose in tissues and are thus diluted out due to in vivo cell divisions of the SDAAS strain. The best presently validated Family B strains are χ12707 (with the genotype: ΔP_{asdA55}::TT araC P_{araBAD} asd Δalr-3 ΔP_{dadB99}::TT P_{rhaBAD} dadB ΔfliC180 ΔpagP81::Pₗₚₚ lpxE ΔpagL7 ΔlpxR9 Δ(hin-fljBA)-219 ΔarnT6 ΔeptA4 ΔsifA26 ΔwbaP45 ΔrecA62) and χ12708 (with the genotype: ΔP_{asdA77}::TT P_{rhaBAD1} asd Δalr-3 ΔP_{dadB66}::TT araC P_{araBAD} dadB ΔfliC180 ΔpagP81::Pₗₚₚ lpxE ΔpagL7 ΔlpxR9 Δ(hin-fljBA)-219 ΔarnT6 ΔeptA4 ΔsifA26 ΔwbaP45 ΔrecA62). These two strains only differ in whether synthesis of the AsdA or DadB enzymes are dependent on presence of arabinose or rhamnose.

Since the improved PIESV vector strains described in Examples 4, 5 and 6 are designed to persist for sufficient time to enable induction of memory mucosal, systemic and cellular immune responses, the co-administration of an SDAAS strain at the time of vaccination with a PIESV construct will rapidly induce innate immune response to enhance the induction of superior levels and durations of immunity. Such studies are currently in progress with χ12688 with vectors pG8R369 encoding S. pneumoniae PspA and PlyA protective antigens and pG8R371 encoding the PhtD antigen (see Example 16).

### Example 21. Display Attributes in PIESV Constructs that Enable Differentiation of Infected from Vaccinated Animals (DIVA)

In farm animals produced for international trade of meat products, it is important to be able to distinguish animals infected with a pathogen from those vaccinated to prevent infection by that pathogen. Thus, it is advantageous to be able to distinguish vaccine strains from the wild-type pathogens from which they were derived and also to determine whether immune responses in slaughtered animals are due to infection versus vaccination. To achieve this, it is best to both inactivate display of a trait always exhibited by the wild-type pathogen and replace it with a readily identifiable trait not displayed by that pathogen. We thus deleted the phs gene encoding the enzyme for synthesis of H₂S that is produced by all Salmonella and is readily seen by formation of black colonies on several different types of indicator agar plates. As an identifiable marker not made by Salmonella strains we can replace the phs gene with a sequence encoding β-galactosidase specified by the lacZ gene, alkaline phosphatase specified by the phoA gene or the green fluorescent protein (GFP) encoded by the egfp gene or the mCherry marker. χ12528 was derived from the wild-type S. typhimurium UK-1 strain χ3761 using the suicide vector pG8R127 (and pG8R125 was also constructed to add the mCherry marker gene in lieu of the phs gene) to generate the ΔphsA19 and ΔphsA19::mCherry mutations. The coding sequence for any desired marker produce such as LacZ, PhoA, mCherry or GFP can be inserted into the suicide vector and used to add the DIVA construct into any of the PIESV and SDAAS strains described above.

### Example 22. Attributes that Facilitate Manufacture of PIESV Constructs Using Fermenter Technologies

The growth properties of all the PIESV and SDAAS strains described are very suitable for commercialization by propagation in large-volume fermenters. Often the media to propagate the strains need to be augmented to maximize attainment of densities of at least 10¹⁰ CFU per ml. This also usually also requires monitoring and adjusting pH, aeration and supply of a metabolizable sugar. All of the improved PIESV stains described herein have deletion mutations precluding their catabolism of the sugars arabinose and rhamnose required to enable their viable growth. Since arabinose and rhamnose are costly media ingredients, the inclusion of these deletions mutations lowers the cost for vaccine manufacture. The cultures grown in fermenters can be harvested by filtration or centrifugation, lyophilized in a medium that enhances the shelf life and bottled in sterile vials. The lyophilized vaccine can then be reconstituted at the time and place of vaccination. Administration can be by course spray to newly hatched chicks in the hatchery and by needle-free oral, intranasal and/or intraocular routes for mucosal administration.

In addition, these vaccines can be administered by parenteral routes although this imposes the costs for needles and handling. Salmonella-based vaccines previously made by us using these procedures are commercially distributed. These include Megan Vac™ and Megan Egg™ distributed by Elanco, Argus SC/ST™ marketed by Merck Animal Health and AVERT™ marketed by Huvepharma.

The above description is provided as an aid in examining particular aspects of the invention, and represents only certain embodiments and explanations of embodiments. The examples are in no way meant to be limiting of the invention scope. The materials and methods provided below are those which were used in performing the examples that follow. It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

### REFERENCES

1. Curtiss R, III. 1986. 1984 Kreshover lecture. Genetic analysis of Streptococcus mutans virulence and prospects for an anticaries vaccine. J Dent Res 65:1034-1045.
2. Curtiss R, III. May 2, 1994 1999. Recombinant avirulent bacterial antigen delivery system. US.
3. Curtiss R, III, Kelly S M. 1987. Salmonella typhimurium deletion mutants lacking adenylate cyclase and cyclic AMP receptor protein are avirulent and immunogenic. Infect Immun 55:3035-3043.
4. Curtiss R, III. Oct. 22, 1992 1995. Avirulent microbes and uses therefor.
5. Galan J E, Curtiss R, III. 1989. Cloning and molecular characterization of genes whose products allow Salmonella typhimurium to penetrate tissue culture cells. Proc Natl Acad Sci USA 86:6383-6387.
6. Curtiss R, III, Galan J. 1995. Vaccines containing avirulent phop-type microorganisms.
7. Nakayama K, Kelly S M, Curtiss R, III 1988. Construction of an Asd+ expression-cloning Vector: stable maintenance and high level expression of cloned genes in a Salmonella vaccine strain. Nat Biotech 6:693-697.
8. Curtiss R, III. Mar. 10, 1995 1997. Selective maintenance of a recombinant gene in a population of vaccine cells.
9. Curtiss R, III. Oct. 11, 2000 2005. Functional balanced-lethal host-vector systems. US.
10. Schödel F, Kelly S M, Peterson D L, Milich D R, Curtiss R, III. 1994. Hybrid hepatitis B virus core-pre-S proteins synthesized in avirulent Salmonella typhimurium and Salmonella typhi for oral vaccination. Infect Immun 62:1669-1676.
11. Kang H Y, Srinivasan J, Curtiss R, III. 2002. Immune responses to recombinant pneumococcal PspA antigen delivered by live attenuated Salmonella enterica serovar Typhimurium vaccine. Infect Immun 70:1739-1749.
12. Galen J E, Curtiss R, III. 2014. The delicate balance in genetically engineering live vaccines. Vaccine 32:4376-4385.
13. Li Y, Wang S, Scarpellini G, Gunn B, Xin W, Wanda S Y, Roland K L, Curtiss R, III. 2009. Evaluation of new generation Salmonella enterica serovar Typhimurium vaccines with regulated delayed attenuation to induce immune responses against PspA. Proc Natl Acad Sci USA 106:593-598.
14. Curtiss R, III., Wanda S Y, Gunn B M, Zhang X, Tinge S A, Ananthnarayan V, Mo H, Wang S, Kong W. 2009. Salmonella enterica serovar Typhimurium strains with regulated delayed attenuation in vivo. Infect Immun 77:1071-1082.
15. Curtiss R, III. Apr. 15, 2003 2012. Regulated attenuation of live vaccines to enhance cross-protective immunogenicity. U.S. Pat. No. 8,133,493.
16. Wang S, Li Y, Scarpellini G, Kong W, Shi H, Baek C H, Gunn B, Wanda S Y, Roland K L, Zhang X, Senechal-Willis P, Curtiss R, III. 2010. Salmonella vaccine vectors displaying delayed antigen synthesis in vivo to enhance immunogenicity. Infect Immun 78:3969-3980.
17. Curtiss R, III, Wang S, Wanda S-Y, Kong W. Feb. 10, 2016 2018. Regulated expression of antigen and/or regulated attenuation to enhance vaccine immunogenicity and/or safety.
18. Kong W, Wanda S Y, Zhang X, Bollen W, Tinge S A, Roland K L, Curtiss R, III. 2008. Regulated programmed lysis of recombinant Salmonella in host tissues to release protective antigens and confer biological containment. Proc Natl Acad Sci USA 105:9361-9366.
19. Ameiss K, Ashraf S, Kong W, Pekosz A, Wu W H, Milich D, Billaud J N, Curtiss R, III. 2010. Delivery of woodchuck hepatitis virus-like particle presented influenza M2e by recombinant attenuated Salmonella displaying a delayed lysis phenotype. Vaccine 28:6704-6713.
20. Ashraf S, Kong W, Wang S, Yang J, Curtiss R, III. 2011. Protective cellular responses elicited by vaccination with influenza nucleoprotein delivered by a live recombinant attenuated Salmonella vaccine. Vaccine 29:3990-4002.
21. Juarez-Rodriguez M D, Yang J, Kader R, Alamuri P, Curtiss R, III, Clark-Curtiss J E. 2012. Live attenuated Salmonella vaccines displaying regulated delayed lysis and delayed antigen synthesis to confer protection against Mycobacterium tuberculosis. Infect Immun 80:815-831.
22. Jiang Y, Mo H, Willingham C, Wang S, Park J Y, Kong W, Roland K L, Curtiss R, III. 2015. Protection Against Necrotic Enteritis in Broiler Chickens by Regulated Delayed Lysis Salmonella Vaccines. Avian Dis 59:475-485.
23. Kong W, Wang X, Fields E, Okon B, Jenkins M C, Wilkins G, Brovold M, Golden T, Gonzales A, Golden G, Clark-Curtiss J E, Curtiss R, III. 2020. Mucosal delivery of a self-destructing Salmonella-based vaccine inducing immunity against Eimeria Manuscript format and headings for Avian Diseases. Avian Diseases In press:null.
24. Li Y, Wang S, Xin W, Scarpellini G, Shi Z, Gunn B, Roland K L, Curtiss R, III. 2008. A sopB deletion mutation enhances the immunogenicity and protective efficacy of a heterologous antigen delivered by live attenuated Salmonella enterica vaccines. Infect Immun 76:5238-5246.
25. Wang S, Shi H, Li Y, Shi Z, Zhang X, Baek C H, Mothershead T, Curtiss R, III. 2013. A colanic acid operon deletion mutation enhances induction of early antibody responses by live attenuated Salmonella vaccine strains. Infect Immun 81:3148-3162.
26. Rowley D. 1966. Phagocytosis and immunity. The central role of phagocytosis in immune reactions. Experientia 22:1-5.
27. Reeves P. 1995. Role of O-antigen variation in the immune response. Trends Microbiol 3:381-386.
28. Fields P I, Swanson R V, Haidaris C G, Heffron F. 1986. Mutants of Salmonella typhimurium that cannot survive within the macrophage are avirulent. Proc Natl Acad Sci USA 83:5189-5193.
29. Kawli T, He F, Tan M W. 2010. It takes nerves to fight infections: insights on neuro-immune interactions from C. elegans. Dis Model Mech 3:721-731.
30. Zhang W, Chen J, Keyhani N O, Jin K, Wei Q, Xia Y. 2017. Central Nervous System Responses of the Oriental migratory, Locusta migratoria manilensis, to Fungal Infection. Sci Rep 7:10340.
31. Neuman H, Debelius J W, Knight R, Koren O. 2015. Microbial endocrinology: the interplay between the microbiota and the endocrine system. FEMS Microbiol Rev 39:509-521.
32. Ahmed E, A I-Moghazy M. 2020. Microbial Endocrinology: Interaction of the Microbial Hormones with the Host. Biomedical Journal of Scientific & Technical Research 24:18052-18054.
33. Curtiss R, III., Porter S B, Munson M, Tinge S A, Hassan J O, Gentry-Weeks C, Kelly S M. 1991. Nonrecombinant and recombinant avirulent Salmonella live vaccines for poultry, p 169-198. In Blankenship L C, Bailey JHS, Cox N A, Stern N J, Meinersmann R J (ed), Colonization control of human bacterial enteropathogens in poultry. Academic Press New York
34. Zhang X, Kelly S M, Bollen W S, Curtiss R, III. 1997. Characterization and immunogenicity of Salmonella typhimurium S L 344 and UK-1 Δcrp and Δcdt deletion mutants. Infect Immun 65:5381-5387.
35. Sanapala S, Mosca L, Wang S, Curtiss R, III 2018. Comparative evaluation of Salmonella vaccines derived from UK-1 and 14028S submitted.
36. Bertani G. 1951. Studies on lysogenesis. I. The mode of phage liberation by lysogenic Escherichia coli. J Bacteriol 62:293-300.
37. Sambrook J, Russell D W. 2001. Molecular cloning: a laboratory manual, 3rd ed. Cold Spring Harbor Laboratory Press, Cold Spring Harbor, N.Y.
38. McDowall K J, Kaberdin V R, Wu S W, Cohen S N, Lin-Chao S. 1995. Site-specific RNase E cleavage of oligonucleotides and inhibition by stem-loops. Nature 374:287-290.
39. Lin-Chao S, Wong T T, McDowall K J, Cohen S N. 1994. Effects of nucleotide sequence on the specificity of rne-dependent and RNase E-mediated cleavages of RNA I encoded by the pBR322 plasmid. J Biol Chem 269:10797-10803.
40. Schmieger H. 1972. Phage P22-mutants with increased or decreased transduction abilities. Mol Gen Genet 119:75-88.
41. Schmieger H, Backhaus H. 1976. Altered cotransduction frequencies exhibited by HT-mutants of Salmonella-phage P22. Mol Gen Genet 143:307-309.
42. Kang H Y, Dozois C M, Tinge S A, Lee T H, Curtiss R, III. 2002. Transduction-mediated transfer of unmarked deletion and point mutations through use of counterselectable suicide vectors. J Bacteriol 184:307-312.
43. Edwards R A, Keller L H, Schifferli D M. 1998. Improved allelic exchange vectors and their use to analyze 987P fimbria gene expression. Gene 207:149-157.
44. Quandt J, Hynes M F. 1993. Versatile suicide vectors which allow direct selection for gene replacement in gram-negative bacteria. Gene 127:15-21.
45. Roland K, Curtiss R, III., Sizemore D. 1999. Construction and evaluation of a Δcya Δcrp Salmonella typhimurium strain expressing avian pathogenic Escherichia coli078 LPS as a vaccine to prevent airsacculitis in chickens. Avian Dis 43:429-441.
46. Shi H, Santander J, Brenneman K E, Wanda S Y, Wang S, Senechal P, Sun W, Roland K L, Curtiss R, III. 2010. Live recombinant Salmonella Typhi vaccines constructed to investigate the role of rpoS in eliciting immunity to a heterologous antigen. PLoS One 5:e11142.
47. Wilmes-Riesenberg M R, Bearson B, Foster J W, Curtiss R, III. 1996. Role of the acid tolerance response in virulence of Salmonella typhimurium. Infect Immun 64:1085-1092.
48. Murray G L, Attridge S R, Morona R. 2005. Inducible serum resistance in Salmonella typhimurium is dependent on wZZfepE-regulated very long 0 antigen chains. Microbes Infect 7:1296-1304.
49. Hitchcock P J, Brown™. 1983. Morphological heterogeneity among Salmonella lipopolysaccharide chemotypes in silver-stained polyacrylamide gels. J Bacteriol 154:269-277.
50. Haile L A, Puig M, Kelley-Baker L, Verthelyi D. 2015. Detection of innate immune response modulating impurities in therapeutic proteins. PLoS One 10:e0125078.
51. Su H, Liu Q, Wang S, Curtiss R, 3rd, Kong Q. 2019. Regulated Delayed Shigella flexneri2a O-antigen Synthesis in Live Recombinant Salmonella enterica Serovar Typhimurium Induces Comparable Levels of Protective Immune Responses with Constitutive Antigen Synthesis System. Theranostics 9:3565-3579.
52. Maino V C, Suni M A, Ruitenberg J J. 1995. Rapid flow cytometric method for measuring lymphocyte subset activation. Cytometry 20:127-133.
53. Maino V C. 1998. Rapid assessment of antigen induced cytokine expression in memory T cells by flow cytometry. Vet Immunol Immunopathol 63:199-207.
54. Maino V C, Picker L J. 1998. Identification of functional subsets by flow cytometry: intracellular detection of cytokine expression. Cytometry 34:207-215.
55. Maecker H T, Dunn H S, Suni M A, Khatamzas E, Pitcher C J, Bunde T, Persaud N, Trigona W, Fu T M, Sinclair E, Bredt B M, McCune J M, Maino V C, Kern F, Picker L J. 2001. Use of overlapping peptide mixtures as antigens for cytokine flow cytometry. J Immunol Methods 255:27-40.
56. Maino V C, Maecker H T. 2004. Cytokine flow cytometry: a multiparametric approach for assessing cellular immune responses to viral antigens. Clin Immunol 110:222-231.
57. McKinnon K M. 2018. Flow Cytometry: An Overview. Curr Protoc Immunol 120:5 1 1-51 11.
58. Galan J E, Nakayama K, Curtiss R, III. 1990. Cloning and characterization of the asd gene of Salmonella typhimurium: use in stable maintenance of recombinant plasmids in Salmonella vaccine strains. Gene 94:29-35.
59. Black S, Wright N G. 1955. Aspartic R-semialdehyde dehydrogenase and aspartic R-semialdehyde. J Biol Chem 213:39-50.
60. Xin W, Wanda S Y, Zhang X, Santander J, Scarpellini G, Ellis K, Alamuri P, Curtiss R, III. 2012. The Asd⁺-DadB⁺ dual-plasmid system offers a novel means to deliver multiple protective antigens by a recombinant attenuated Salmonella vaccine. Infect Immun 80:3621-3633.
61. Lin H C, Lei S P, Studnicka G, Wilcox G. 1985. The araBAD operon of Salmonella typhimurium LT2. III. Nucleotide sequence of araD and its flanking regions, and primary structure of its product, L-ribulose-5-phosphate 4-epimerase. Gene 34:129-134.
62. Lin H C, Lei S P, Wilcox G. 1985. The araBAD operon of Salmonella typhimurium LT2. II. Nucleotide sequence of araA and primary structure of its product, L-arabinose isomerase. Gene 34:123-128.
63. Lin H C, Lei S P, Wilcox G. 1985. The araBAD operon of Salmonella typhimurium LT2. I. Nucleotide sequence of araB and primary structure of its product, ribulokinase. Gene 34:111-122.
64. Lee J H, Nishitani J, Wilcox G. 1984. Genetic characterization of Salmonella typhimurium LT2 ara mutations. J Bacteriol 158:344-346.
65. Horwitz A H, Heffernan L, Morandi C, Lee J H, Timko J, Wilcox G. 1981. DNA sequence of the araBAD-araC controlling region in Salmonella typhimurium LT2. Gene 14:309-319.
66. Moralejo P, Egan S M, Hidalgo E, Aguilar J. 1993. Sequencing and characterization of a gene cluster encoding the enzymes for L-rhamnose metabolism in Escherichia coli. J Bacteriol 175:5585-5594.
67. Egan S M, Schleif R F. 1993. A regulatory cascade in the induction of rhaBAD. J Mol Biol 234:87-98.
68. Kong O, Six D A, Roland K L, Liu O, Gu L, Reynolds C M, Wang X, Raetz C R, Curtiss R, III. 2011. Salmonella synthesizing 1-dephosphorylated lipopolysaccharide exhibits low endotoxic activity while retaining its immunogenicity. J Immunol 187:412-423.
69. Kong O, Six D A, Liu O, Gu L, Wang S, Alamuri P, Raetz C R, Curtiss R, III. 2012. Phosphate groups of lipid A are essential for Salmonella enterica serovar Typhimurium virulence and affect innate and adaptive immunity. Infect Immun 80:3215-3224.
70. Lee H, Hsu F F, Turk J, Groisman E A. 2004. The PmrA-regulated pmrC gene mediates phosphoethanolamine modification of lipid A and polymyxin resistance in Salmonella enterica. J Bacteriol 186:4124-4133.
71. Trent M, Raetz C. 2002. Cloning of EptA, the lipid A phosphoethanolamine transferase associated with polymyxin resistance. J Endotoxin Res 8:159.
72. Trent M S, Ribeiro A A, Lin S, Cotter R J, Raetz C R. 2001. An inner membrane enzyme in Salmonella and Escherichia colithat transfers 4-amino-4-deoxy-L-arabinose to lipid A: induction on polymyxin-resistant mutants and role of a novel lipid-linked donor. J Biol Chem 276:43122-43131.
73. lino T, Komeda Y, Kutsukake K, Macnab R M, Matsumura P, Parkinson J S, Simon M I, Yamaguchi S. 1988. New unified nomenclature for the flagellar genes of Escherichia coli and Salmonella typhimurium. Microbiol Rev 52:533-535.
74. lino T. 1977. Genetics of structure and function of bacterial flagella. Annu Rev Genet 11:161-182.
75. lino T. 1969. Genetics and chemistry of bacterial flagella. Bacteriol Rev 33:454-475.
76. Liu O, Liu O, Yi J, Liang K, Hu B, Zhang X, Curtiss R, III, Kong Q. 2016. Outer membrane vesicles from flagellin-deficient Salmonella enterica serovar Typhimurium induce cross-reactive immunity and provide cross-protection against heterologous Salmonella challenge. Sci Rep 6:34776.
77. Zieg J, Simon M. 1980. Analysis of the nucleotide sequence of an invertible controlling element. Proc Natl Acad Sci USA 77:4196-4200.
78. Silverman M, Simon M. 1980. Phase variation: genetic analysis of switching mutants. Cell 19:845-854.
79. Silverman M, Zieg J, Simon M. 1979. Flagellar-phase variation: isolation of the rh1 gene. J Bacteriol 137:517-523.
80. Kutsukake K, lino T. 1980. Inversions of specific DNA segments in flagellar phase variation of Salmonella and inversion systems of bacteriophages P1 and Mu. Proc Natl Acad Sci USA 77:7338-7341.
81. Bonifield H R, Hughes K T. 2003. Flagellar phase variation in Salmonella enterica is mediated by a posttranscriptional control mechanism. J Bacteriol 185:3567-3574.
82. Kutsukake K, lino T. 1980. A trans-acting factor mediates inversion of a specific DNA segment in flagellar phase variation of Salmonella. Nature 284:479-481.
83. Yamamoto S, Kutsukake K. 2006. FIjA-mediated posttranscriptional control of phase 1 flagellin expression in flagellar phase variation of Salmonella enterica serovar Typhimurium. J Bacteriol 188:958-967.
84. Römling U, Bian Z, Hammar M, Sierralta W D, Normark S. 1998. Curli fibers are highly conserved between Salmonella typhimurium and Escherichia coliwith respect to operon structure and regulation. J Bacteriol 180:722-731.
85. Laniewski P, Baek C H, Roland K L, Curtiss R, III. 2017. Analysis of Spleen-Induced Fimbria Production in Recombinant Attenuated Salmonella enterica Serovar Typhimurium Vaccine Strains. MBio 8:e01 189-01117.
86. Freudl R, Cole S T. 1983. Cloning and molecular characterization of the ompA gene from Salmonella typhimurium. Eur J Biochem 134:497-502.
87. Norris F A, Wilson M P, Wallis T S, Galyov E E, Majerus P W. 1998. SopB, a protein required for virulence of Salmonella dublin, is an inositol phosphate phosphatase. Proc Natl Acad Sci USA 95:14057-14059.
88. Reis B P, Zhang S, Tsolis R M, Baumler A J, Adams L G, Santos R L. 2003. The attenuated sopB mutant of Salmonella enterica serovar Typhimurium has the same tissue distribution and host chemokine response as the wild type in bovine Peyer's patches. Vet Microbiol 97:269-277.
89. Zhang S, Santos R L, Tsolis R M, Stender S, Hardt W D, Baumler A J, Adams L G. 2002. The Salmonella enterica serotype typhimurium effector proteins SipA, SopA, SopB, SopD, and SopE2 act in concert to induce diarrhea in calves. Infect Immun 70:3843-3855.
90. Collins L V, Attridge S, Hackett J. 1991. Mutations at rfc or pmiattenuate Salmonella typhimurium virulence for mice. Infect Immun 59:1079-1085.
91. Kong O, Yang J, Liu O, Alamuri P, Roland K L, Curtiss R, III. 2011. Effect of deletion of genes involved in lipopolysaccharide core and O-antigen synthesis on virulence and immunogenicity of Salmonella enterica serovar Typhimurium. Infect Immun 79:4227-4239.
92. Stevenson G, Andrianopoulos K, Hobbs M, Reeves P R. 1996. Organization of the Escherichia coli K-12 gene cluster responsible for production of the extracellular polysaccharide colanic acid. J Bacteriol 178:4885-4893.
93. Whitfield C. 2006. Biosynthesis and assembly of capsular polysaccharides in Escherichia coli. Annu Rev Biochem 75:39-68.
94. Pizarro-Cerda J, Tedin K. 2004. The bacterial signal molecule, ppGpp, regulates Salmonella virulence gene expression. Mol Microbiol 52:1827-1844.
95. Torok I, Kari C. 1980. Accumulation of ppGpp in a relA mutant of Escherichia coli during amino acid starvation. J Biol Chem 255:3838-3840.
96. Ahmer B M, Tran M, Heffron F. 1999. The virulence plasmid of Salmonella typhimurium is self-transmissible. J Bacteriol 181:1364-1368.
97. Cohen A, Laban A. 1983. Plasmidic recombination in Escherichia coli K-12: the role of recFgene function. Mol Gen Genet 189:471-474.
98. Kolodner R, Fishel R A, Howard M. 1985. Genetic recombination of bacterial plasmid DNA: effect of RecF pathway mutations on plasmid recombination in Escherichia coli. J Bacteriol 163:1060-1066.
99. Zhang X, Wanda S Y, Brenneman K, Kong W, Roland K, Curtiss R, III. 2011. Improving Salmonella vector with rec mutation to stabilize the DNA cargoes. BMC Microbiol 11:31.
100. Cohen S S, Barner H D. 1954. Studies on Unbalanced Growth in Escherichia coli. Proc Natl Acad Sci USA 40:885-893.
101. Hickson I D, Atkinson K E, Emmerson P T. 1982. Molecular cloning and amplification of the gene for thymidylate synthetase of E. coli. Gene 18:257-260.
102. Bazill G W. 1967. Lethal unbalanced growth in bacteria. Nature 216:346-349.
103. Khodursky A, Guzman E C, Hanawalt P C. 2015. Thymineless Death Lives On: New Insights into a Classic Phenomenon. Annu Rev Microbiol 69:247-263.
104. Belfort M, Maley G, Pedersen-Lane J, Maley F. 1983. Primary structure of the Escherichia coli thyA gene and its thymidylate synthase product. Proc Natl Acad Sci USA 80:4914-4918.
105. Beuzon C R, Meresse S, Unsworth K E, Ruiz-Albert J, Garvis S, Waterman S R, Ryder T A, Boucrot E, Holden D W. 2000. Salmonella maintains the integrity of its intracellular vacuole through the action of SifA. EMBO J 19:3235-3249.
106. Ohlson M B, Huang Z, Alto N M, Blanc M P, Dixon J E, Chai J, Miller S I. 2008. Structure and function of Salmonella SifA indicate that its interactions with SKIP, SseJ, and RhoA family GTPases induce endosomal tubulation. Cell Host Microbe 4:434-446.
107. Kong W, Brovold M, Koeneman B A, Clark-Curtiss J, Curtiss R, III. 2012. Turning self-destructing Salmonella into a universal DNA vaccine delivery platform. Proc Natl Acad Sci USA 109:19414-19419.
108. Griffin A J, McSorley S J. 2011. Generation of Salmonella-specific Th1 cells requires sustained antigen stimulation. Vaccine 29:2697-2704.
109. Pando J M, Karlinsey J E, Lara J C, Libby S J, Fang F C. 2017. The Rcs-Regulated Colanic Acid Capsule Maintains Membrane Potential in Salmonella enterica serovar Typhimurium. mBio 8.
110. Mao Y, Doyle M P, Chen J. 2001. Insertion mutagenesis of wca reduces acid and heat tolerance of enterohemorrhagic Escherichia coli0157:H7. J Bacteriol 183:3811-3815.
111. Ophir T, Gutnick D L. 1994. A role for exopolysaccharides in the protection of microorganisms from desiccation. Appl Environ Microbiol 60:740-745.
112. Chen J, Lee S M, Mao Y. 2004. Protective effect of exopolysaccharide colanic acid of Escherichia coli0157:H7 to osmotic and oxidative stress. Int J Food Microbiol 93:281-286.
113. Ranjit D K, Young K D. 2016. Colanic Acid Intermediates Prevent De Novo Shape Recovery of Escherichia coli Spheroplasts, Calling into Question Biological Roles Previously Attributed to Colanic Acid. J Bacteriol 198:1230-1240.
114. Adcox H E, Vasicek E M, Dwivedi V, Hoang K V, Turner J, Gunn J S. 2016. Salmonella Extracellular Matrix Components Influence Biofilm Formation and Gallbladder Colonization. Infect Immun 84:3243-3251.
115. Crawford R W, Gibson D L, Kay W W, Gunn J S. 2008. Identification of a bile-induced exopolysaccharide required for Salmonella biofilm formation on gallstone surfaces. Infect Immun 76:5341-5349.
116. Gibson D L, White ΔP, Snyder S D, Martin S, Heiss C, Azadi P, Surette M, Kay W W. 2006. Salmonella produces an O-antigen capsule regulated by AgfD and important for environmental persistence. J Bacteriol 188:7722-7730.
117. Irvine K L, Gangloff M, Walsh C M, Spring D R, Gay N J, Bryant C E. 2014. Identification of key residues that confer Rhodobacter sphaeroides LPS activity at horse TLR4/MD-2. PLoS One 9:e98776.
118. Lohmann K L, Vandenplas M L, Barton M H, Bryant C E, Moore J N. 2007. The equine TLR4/MD-2 complex mediates recognition of lipopolysaccharide from Rhodobacter sphaeroides as an agonist. J Endotoxin Res 13:235-242.
119. Lohmann K L, Vandenplas M, Barton M H, Moore J N. 2003. Lipopolysaccharide from Rhodobacter sphaeroides is an agonist in equine cells. J Endotoxin Res 9:33-37.
120. Walsh C, Gangloff M, Monie T, Smyth T, Wei B, McKinley T J, Maskell D, Gay N, Bryant C. 2008. Elucidation of the MD-2/TLR4 interface required for signaling by lipid IVa. J Immunol 181:1245-1254.
121. van Miert A S, Frens J. 1968. The reaction of different animal species to bacterial pyrogens. Zentralbl Veterinarmed A 15:532-543.
122. De Boever S, Beyaert R, Vandemaele F, Baert K, Duchateau L, Goddeeris B, De Backer P, Croubels S. 2008. The influence of age and repeated lipopolysaccharide administration on body temperature and the concentration of interleukin-6 and IgM antibodies against lipopolysaccharide in broiler chickens. Avian Pathol 37:39-44.
123. Gunn B M, Wanda S Y, Burshell D, Wang C, Curtiss R, III. 2010. Construction of recombinant attenuated Salmonella enterica serovar Typhimurium vaccine vector strains for safety in newborn and infant mice. Clin Vaccine Immunol 17:354-362.
124. Link C, Ebensen T, Standner L, Dejosez M, Reinhard E, Rharbaoui F, Guzman C A. 2006. An SopB-mediated immune escape mechanism of Salmonella enterica can be subverted to optimize the performance of live attenuated vaccine carrier strains. Microbes Infect 8:2262-2269.
125. Lockman H A, Curtiss R, III. 1992. Virulence of non-type 1-fimbriated and nonfimbriated nonflagellated Salmonella typhimurium mutants in murine typhoid fever. Infect Immun 60:491-496.
126. Lu Y, Swartz J R. 2016. Functional properties of flagellin as a stimulator of innate immunity. Sci Rep 6:18379.
127. Römling U, Sierralta W D, Eriksson K, Normark S. 1998. Multicellular and aggregative behaviour of Salmonella typhimurium strains is controlled by mutations in the agfD promoter. Mol Microbiol 28:249-264.
128. Römling U, Rohde M, Olsen A, Normark S, Reinkoster J. 2000. AgfD, the checkpoint of multicellular and aggregative behaviour in Salmonella typhimurium regulates at least two independent pathways. Mol Microbiol 36:10-23.
129. Simm R, Ahmad I, Rhen M, Le Guyon S, Römling U. 2014. Regulation of biofilm formation in Salmonella enterica serovar Typhimurium. Future Microbiol 9:1261-1282.
130. Muralinath M, Kuehn M J, Roland K L, Curtiss R, III. 2011. Immunization with Salmonella enterica serovar Typhimurium-derived outer membrane vesicles delivering the pneumococcal protein PspA confers protection against challenge with Streptococcus pneumoniae. Infect Immun 79:887-894.
131. Tauxe R V. 1991. Salmonella: A Postmodern Pathogen. J Food Prot 54:563-568.
132. Kendall P, Medeiros L C, Hillers V, Chen G, DiMascola S. 2003. Food handling behaviors of special importance for pregnant women, infants and young children, the elderly, and immune-compromised people. J Am Diet Assoc 103:1646-1649.
133. Collaborators GBDN-TSID. 2019. The global burden of non-typhoidal Salmonella invasive disease: a systematic analysis for the Global Burden of Disease Study 2017. Lancet Infect Dis 19:1312-1324.
134. Ao T T, Feasey N A, Gordon M A, Keddy K H, Angulo F J, Crump J A. 2015. Global burden of invasive nontyphoidal Salmonella disease, 2010. Emerg Infect Dis 21.
135. Vugia D J, Samuel M, Farley M M, Marcus R, Shiferaw B, Shallow S, Smith K, Angulo F J, Emerging Infections Program FoodNet Working G. 2004. Invasive Salmonella infections in the United States, FoodNet, 1996-1999: incidence, serotype distribution, and outcome. Clin Infect Dis 38 Suppl 3:S149-156.
136. Feasey N A, Dougan G, Kingsley R A, Heyderman R S, Gordon M A. 2012. Invasive non-typhoidal Salmonella disease: an emerging and neglected tropical disease in Africa. Lancet 379:2489-2499.
137. Osborn M J, Wu H C. 1980. Proteins of the outer membrane of gram-negative bacteria. Annu Rev Microbiol 34:369-422.
138. Braun V, Hantke K. 1974. Biochemistry of bacterial cell envelopes. Annu Rev Biochem 43:89-121.
139. Sha J, Fadl A A, Klimpel G R, Niesel D W, Popov V L, Chopra A K. 2004. The two murein lipoproteins of Salmonella enterica serovar Typhimurium contribute to the virulence of the organism. Infect Immun 72:3987-4003.
140. Lee J S, Jung I D, Lee C M, Park J W, Chun S H, Jeong S K, Ha T K, Shin Y K, Kim D J, Park Y M. 2010. Outer membrane protein a of Salmonella enterica serovar Typhimurium activates dendritic cells and enhances Th1 polarization. BMC Microbiol 10:263.
141. Puohiniemi R, Karvonen M, Vuopio-Varkila J, Muotiala A, Helander I M, Sarvas M. 1990. A strong antibody response to the periplasmic C-terminal domain of the OmpA protein of Escherichia coli is produced by immunization with purified OmpA or with whole E. colior Salmonella typhimurium bacteria. Infect Immun 58:1691-1696.
142. Singh S P, Williams Y U, Miller S, Nikaido H. 2003. The C-terminal domain of Salmonella enterica serovar typhimurium OmpA is an immunodominant antigen in mice but appears to be only partially exposed on the bacterial cell surface. Infect Immun 71:3937-3946.
143. Gonzalez C R, Isibasi A, Ortiz-Navarrete V, Paniagua J, Garcia J A, Blanco F, Kumate J. 1993. Lymphocytic proliferative response to outer-membrane proteins isolated from Salmonella. Microbiol Immunol 37:793-799.
144. Udhayakumar V, Muthukkaruppan V R. 1987. Protective immunity induced by outer membrane proteins of Salmonella typhimurium in mice. Infect Immun 55:816-821.
145. Jang M J, Kim J E, Chung Y H, Lee W B, Shin Y K, Lee J S, Kim D, Park Y M. 2011. Dendritic cells stimulated with outer membrane protein A (OmpA) of Salmonella typhimurium generate effective anti-tumor immunity. Vaccine 29:2400-2410.
146. Krishnan S, Prasadarao N V. 2012. Outer membrane protein A and OprF: versatile roles in Gram-negative bacterial infections. FEBS J 279:919-931.
147. Chatfield S N, Dorman C J, Hayward C, Dougan G. 1991. Role of ompR-dependent genes in Salmonella typhimurium virulence: mutants deficient in both ompC and ompFare attenuated in vivo. Infect Immun 59:449-452.
148. Dorman C J, Chatfield S, Higgins C F, Hayward C, Dougan G. 1989. Characterization of porin and ompR mutants of a virulent strain of Salmonella typhimurium: ompR mutants are attenuated in vivo. Infect Immun 57:2136-2140.
149. Ipinza F, Collao B, Monsalva D, Bustamante V H, Luraschi R, Alegria-Arcos M, Almonacid D E, Aguayo D, Calderon I L, Gil F, Santiviago C A, Morales E H, Calva E, Saavedra C P. 2014. Participation of the Salmonella OmpD porin in the infection of RAW264.7 macrophages and BALB/c mice. PLoS One 9:e111062.
150. Meyer P N, Wilmes-Riesenberg M R, Stathopoulos C, Curtiss R, III. 1998. Virulence of a Salmonella typhimurium OmpD mutant. Infect Immun 66:387-390.
151. Caldwell A L, Gulig P A. 1991. The Salmonella typhimurium virulence plasmid encodes a positive regulator of a plasmid-encoded virulence gene. J Bacteriol 173:7176-7185.
152. Gulig P A, Danbara H, Guiney D G, Lax A J, Norel F, Rhen M. 1993. Molecular analysis of spv virulence genes of the Salmonella virulence plasmids. Mol Microbiol 7:825-830.
153. Gulig P A, Caldwell A L, Chiodo V A. 1992. Identification, genetic analysis and DNA sequence of a 7.8-kb virulence region of the Salmonella typhimurium virulence plasmid. Mol Microbiol 6:1395-1411.
154. Yoon H, McDermott J E, Porwollik S, McClelland M, Heffron F. 2009. Coordinated regulation of virulence during systemic infection of Salmonella enterica serovar Typhimurium. PLoS Pathog 5:e1000306.
155. Silva C, Puente J L, Calva E. 2017. Salmonella virulence plasmid: pathogenesis and ecology. Pathog Dis doi:10.1093/femspd/ftx070.
156. Gulig P A, Curtiss R, III. 1987. Plasmid-associated virulence of Salmonella typhimurium. Infect Immun 55:2891-2901.
157. Gulig P A, Curtiss R, III. 1988. Cloning and transposon insertion mutagenesis of virulence genes of the 100-kilobase plasmid of Salmonella typhimurium. Infect Immun 56:3262-3271.
158. Stinavage P, Martin L E, Spitznagel J K. 1989. O antigen and lipid A phosphoryl groups in resistance of Salmonella typhimurium LT-2 to nonoxidative killing in human polymorphonuclear neutrophils. Infect Immun 57:3894-3900.
159. Wick M J, Harding C V, Normark S J, Pfeifer J D. 1994. Parameters that influence the efficiency of processing antigenic epitopes expressed in Salmonella typhimurium. Infect Immun 62:4542-4548.
160. Liang-Takasaki C J, Grossman N, Leive L. 1983. Salmonellae activate complement differentially via the alternative pathway depending on the structure of their lipopolysaccharide O-antigen. J Immunol 130:1867-1870.
161. Liang-Takasaki C J, Makela P H, Leive L. 1982. Phagocytosis of bacteria by macrophages: changing the carbohydrate of lipopolysaccharide alters interaction with complement and macrophages. J Immunol 128:1229-1235.
162. Luo Y, Kong Q, Yang J, Golden G, Wanda S Y, Jensen R V, Ernst P B, Curtiss R, III. 2011. Complete genome sequence of the universal killer Salmonella enterica Serovar Typhimurium UK-1 (ATCC 68169). J Bacteriol 193:4035-4036.
163. Luo Y, Kong Q, Yang J, Mitra A, Golden G, Wanda S Y, Roland K L, Jensen R V, Ernst P B, Curtiss R, III. 2012. Comparative genome analysis of the high pathogenicity Salmonella Typhimurium strain UK-1. PLoS One 7:e40645.
164. Xin W, Li Y, Mo H, Roland K L, Curtiss R, III. 2009. PspA family fusion proteins delivered by attenuated Salmonella enterica serovar Typhimurium extend and enhance protection against Streptococcus pneumoniae. Infect Immun 77:4518-4528.
165. Curtiss R, III, Wang S. 3 Aug. 2018 2019. Induction of protective immunity against antigens. USWO 2019/028396 A1.
166. Zekarias B, Mo H, Curtiss R, III. 2008. Recombinant attenuated Salmonella enterica serovar Typhimurium expressing the carboxy-terminal domain of alpha toxin from Clostridium perfringens induces protective responses against necrotic enteritis in chickens. Clin Vaccine Immunol 15:805-816.
167. Wilde S, Jiang Y, Tafoya A M, Horsman J, Yousif M, Vazquez L A, Roland K L. 2019. Salmonella-vectored vaccine delivering three Clostridium perfringens antigens protects poultry against necrotic enteritis. PLoS One 14:e0197721.
168. Wang S, Hofacre C L, Wanda S-Y, Callum JZRA, Nordgren B, Curtiss R. 2021. A triple-sugar regulated Salmonella vaccine protects against Clostridium perfringens-induced necrotic enteritis in broiler chickens. Poultry Science doi:https://doi.orq/10.1016/j.psj.2021.101592:101592.

What is claimed is:

1. An attenuated derivative of a pathogenic Salmonella that comprises one or more genotypic/phenotypic properties of χ12688 and χ12702, or descendants or derivatives thereof.

2. The attenuated derivative of a pathogenic Salmonella of claim 1, wherein the attenuated derivative of a pathogenic Salmonella comprises one or more of the following genetic modifications: ΔP_{murA25}::TT araC P_{araBAD} murA, ΔasdA33, ΔwaaL46, ΔpagL38::TT rhaRS P_{rhaBAD2} waaL2, Δ(wza-wcaM)-8, ΔrelA1123, ΔrecF126, ΔsifA26, ΔaraBAD65::TT, ΔrhaBADSR515, ΔpagP8 or ΔpagP81::Pₗₚₚ lpxE, ΔlpxR9, (pSTUK206 or Δ(traM-traX)-41:: araC P_{araBAD} lacI TT).

3. The attenuated derivative of a pathogenic Salmonella of claim 1, wherein the attenuated derivative of a pathogenic Salmonella comprises one or more of the following genetic modifications: ΔeptA4, ΔarnT6, ΔP_{asdA55}::TT araC P_{araBAD} asd or ΔP_{asdA77}::TT P_{rhaBAD1} asd or ΔP_{asdA88}::TT rhaRS P_{rhaBAD1} asd (in place of ΔasdA33), ΔompA11, ΔsopB1925, ΔpagP81::Pₗₚₚ lpxE (in place of ΔpagP8), ΔstcABCD, ΔP_{stc53}::P_{murA} stcA53, ΔsafABCD, ΔP_{safA55}::P_{murA} safA55, ΔrecA62, Δalr-3, ΔdadB4 or ΔP_{dadB66}::TT araC P_{araBAD} dadB or ΔP_{dadB99}::TT P_{rhaBAD} dadB, ΔfliC180, ΔfliC2426, ΔfljB217, Δ(hin-fljBA)-209, Δ(agfG-agfC)-999, ΔwaaC41, ΔwaaG42, ΔwbaP45, ΔpagL64::TT araC P_{araBAD1} waaL and Δ(traM-traX)-36::araC P_{araBAD} lacI TT or Δ(traM-traX)-37::araC P_{araBAD} lacI TT or Δ(traM-traX)-38::araC P_{araBAD} lacI TT or Δ(traM-traX)-39::araC P_{araBAD} lacI TT or Δ(traM-traX)-40::araC P_{araBAD} lacI TT (in place of Δ(traM-traX)-41::araC P_{araBAD} lacI TT) mutations.

4. The attenuated derivative of a pathogenic Salmonella of claim 1, wherein the attenuated derivative of a pathogenic Salmonella is a recombinant attenuated derivative that further comprises a nucleic acid sequence encoding an immunogen, and wherein the recombinant attenuated derivative of a pathogenic Salmonella synthesizes and delivers the immunogen when inoculated into a subject.

5. The attenuated derivative of claim 4, wherein the immunogen is selected from the group consisting of PspA, PlyA, PhtD, tf, Bp26, Omp31, BIs, lg7/lg12, or Zn/Cu SOD

6. An attenuated derivative of a pathogenic Salmonella that comprises the genotypic/phenotypic properties of χ12704, descendants or derivatives thereof.

7. The attenuated derivative of a pathogenic Salmonella of claim 6, wherein the attenuated derivative of a pathogenic Salmonella comprises one or more of the following genetic modifications: ΔP_{murA25}::TT araC P_{araBAD} murA, ΔasdA33, ΔwaaL46, Δ(wza-wcaM)-8, ΔrelA1123, ΔrecF126, ΔsifA26, ΔmntR28, ΔP_{fur33}::TT araC P_{araBAD} fur, ΔaraBAD65::TT, ΔrhaBADSR515, ΔpagL38::TT rhaRS P_{rhaBAD2} waaL2, (pSTUK206 Δ(traM-traX)-41:: araC P_{araBAD} lacI TT).

8. The attenuated derivative of a pathogenic Salmonella of claim 6, wherein the attenuated derivative of a pathogenic Salmonella comprises one or more of the following genetic modifications: ΔeptA4, ΔarnT6, ΔP_{asdA55}::TT araC P_{araBAD} asd or ΔP_{asdA77}::TT P_{rhaBAD1} asd or ΔP_{asdA88}::TT rhaRS P_{rhaBAD1} asd (in place of ΔasdA27::TT araC P_{araBAD} c2), ΔompA11, ΔsopB1925, ΔpagP81::Pₗₚₚ lpxE (in place of ΔpagP8), ΔstcABCD, ΔP_{stc53}::P_{murA} stcA53, ΔsafABCD, ΔP_{safA55}::P_{murA} safA55, ΔrecA62, Δalr-3, ΔdadB4 or ΔP_{dadB66}::TT araC P_{araBAD} dadB or ΔP_{dadB99}::TT P_{rhaBAD} dadB, P_{mntR44}::TT araC P_{araBAD} mntR, ΔfliC180, ΔfliC2426, ΔfljB217, Δ(hin-fljBA)-209, Δ(agfG-agfC)-999, ΔwaaC41, ΔwaaG42, ΔwbaP45, ΔpagL64::TT araC P_{araBAD1} waaL and Δ(traM-traX)-36::araC P_{araBAD} lacI TT or Δ(traM-traX)-37::araC P_{araBAD} lacI TT or Δ(traM-traX)-38::araC P_{araBAD} lacI TT or Δ(traM-traX)-39::araC P_{araBAD} lacI TT or Δ(traM-traX)-40::araC P_{araBAD} lacI TT (in place of Δ(traM-traX)-41::araC P_{araBAD} lacI TT) mutations.

9. The attenuated derivative of a pathogenic Salmonella of claim 6, wherein the attenuated derivative of a pathogenic Salmonella is a recombinant attenuated derivative that further comprises a nucleic acid sequence encoding an immunogen, and wherein the recombinant attenuated derivative synthesizes and delivers the immunogen when inoculated into a subject.

10. The attenuated derivative of a pathogenic Salmonella of claim 9, wherein the immunogen is selected from the group consisting of S07, BlaSS PlcC, GST-NetB, PelBSS Fba, DsbASS Cbh-6HisTag, OmpASS CpeCMax-6HisTag, M2e,

11. An attenuated derivative of a pathogenic Salmonella that comprises the genotypic/phenotypic properties of χ12706, or descendants or derivatives thereof.

12. The attenuated derivative of a pathogenic Salmonella of claim 11, wherein the attenuated derivative of a pathogenic Salmonella comprises one or more of the following genetic modifications: ΔP_{murA25}::TT araC P_{araBAD} murA, ΔasdA33, ΔwaaL46, ΔpagL38::TT rhaRS P_{rhaBAD2} waaL2, Δ(wza-wcaM)-8. ΔrelA1123, ΔrecF126, ΔsifA26, ΔendA2113, ΔsseL116, ΔtlpA18, ΔrhaBADSR515, ΔaraBAD65::TT.

13. The attenuated derivative of a pathogenic Salmonella of claim 11, wherein the attenuated derivative of a pathogenic Salmonella comprises one or more of the following genetic modifications: ΔeptA4, ΔarnT6, ΔP_{asdA55}::TT araC P_{araBAD} asd or ΔP_{asdA77}::TT P_{rhaBAD1} asd or ΔP_{asdA88}::TT rhaRS P_{rhaBAD1} asd or ΔasdA27::TT araC P_{araBAD} c2 (in place of ΔasdA33), ΔompA11, ΔsopB1925, ΔpagP81::Pₗₚₚ lpxE (in place of ΔpagP8), ΔstcABCD, ΔP_{stc53}::P_{murA} stcA53, ΔsafABCD, ΔP_{safA55}::P_{murA} safA55, ΔrecA62, Δalr-3, ΔdadB4 or ΔP_{dadB66}::TT araC P_{araBAD} dadB or ΔP_{dadB99}::TT P_{rhaBAD} dadB, ΔfliC180, ΔfliC2426, ΔfljB217, Δ(hin-fljBA)-209, Δ(agfG-agfC)-999, ΔwaaC41, ΔwaaG42, ΔwbaP45, and ΔpagL64::TT araC P_{araBAD1} waaL mutations.

14. The attenuated derivative of a pathogenic Salmonella of claim 11, wherein the attenuated derivative of a pathogenic Salmonella is a recombinant attenuated derivative that further comprises a DNA vaccine vector with a nucleic acid sequence encoding an immunogen, and wherein the recombinant attenuated derivative delivers the DNA vaccine to a subject to be expressed in said subject.

15. The attenuated derivative of claim 14, wherein the immunogen comprises WSN HA.

16. (canceled)

17. (canceled)

18. (canceled)

19. (canceled)

20. (canceled)

21. (canceled)

22. (canceled)

23. A recombinant attenuated derivative of a pathogenic Salmonella, wherein the derivative is (i) programmed for regulated delayed antigen synthesis and release by regulated delayed lysis of a WHV core fused to a SARS-CoV-2 segment of the S protein specifying an ACE2 binding domain, wherein the ACE2 binding domain comprises an amino acid sequence of SEQ ID NO: 40 or a fragment thereof of at least 10, 20, 30, 40, or 50 contiguous amino acids; or (ii) programmed for regulated delayed antigen synthesis of a SARS-CoV-2 protein antigen fused to a type 3 secretion system effector to be delivered by both type 3 secretion and by regulated delayed lysis in an inoculated subject, wherein the SARS-CoV-2 protein antigen comprises an amino acid sequence of SEQ ID NO: 39, SEQ ID NO: 41, SEQ ID NO: 53 or a fragment thereof of at least 10, 20, 30, 40, or 50 contiguous amino acids; or (iii) programmed for regulated delayed lysis to release a DNA vaccine that expresses one or more proteins encoded by a SARS-CoV-2 genome, wherein the DNA vaccine comprises at least one nucleic acid sequence of the SARS-CoV-2 genome, wherein the at least one nucleic acid sequence is selected from the group consisting of nucleic acid sequences of SEQ ID NOs: 4-12 or a fragment thereof of at least 10, 20, 30, 40, or 50 contiguous nucleic acids.

24. (canceled)

25. The attenuated derivative of claim 23, wherein the WHV core fused to the SARS-CoV-2 segment of the S protein specifying the ACE2 binding domain comprises an amino acid sequence of SEQ ID NO: 38 or SEQ ID NO: 52 or a fragment thereof of at least 10, 20, 30, 40, or 50 contiguous amino acids.

26. (canceled)

27. (canceled)

28. (canceled)

29. (canceled)

30. (canceled)

31. (canceled)

32. A method of inducing an immune response comprising administering to a subject at least one or more attenuated derivative of claim 1, wherein the immune response is against the immunogen.

33. (canceled)

34. A method of inducing an immune response comprising administering to a subject a recombinant attenuated derivative of claim 23.

35. (canceled)

36. A method of inducing an immune response comprising administering to a subject at least one or more attenuated derivative of claim 11, wherein the immune response is against the immunogen.
